(19) United States
(12) Patent Application Publication
(10) Pub. No.: US 2024/0058464 A1
(43) Pub. Date: Feb. 22, 2024

(54) COMBINATION THERAPY WITH FOR46 FOR CANCER

(71) Applicants: Fortis Therapeutics, Inc., La Jolla, CA (US); The Regents of the University of California, Oakland, CA (US)

(72) Inventors: Bin LIU, Oakland, CA (US); Andrew DORR, La Jolla, CA (US); Jay LICHTER, La Jolla, CA (US); Marc NASOFF, La Jolla, CA (US); Leonard POST, La Jolla, CA (US)

(73) Assignees: Fortis Therapeutics, Inc., La Jolla, CA (US); The Regents of the University of California, Oakland, CA (US)

(21) Appl. No.: 18/260,657

(22) PCT Filed: Jan. 6, 2022
(86) PCT No.: PCT/US2022/011497

§ 371 Date: Jul. 7, 2023

Related U.S. Application Data
(60) Provisional application No. 63/134,896, filed on Jan. 7, 2021.

(51) Int. Cl. A61K 47/68; A61K 31/4196; A61K 31/675; A61K 31/7068; A61K 31/282; A61K 31/58; A61K 39/395; A61K 31/704; A61K 31/44; A61K 31/337; A61K 33/243; A61P 35/00

(52) U.S. Cl. CPC A61K 47/6849; A61K 47/68031; A61K 47/6889; A61K 31/4196; A61K 31/675; A61K 31/7068; A61K 31/282; A61K 31/58; A61K 39/3955; A61K 31/704; A61K 31/44; A61K 31/337; A61K 33/243; A61P 35/00

(57) ABSTRACT

Disclosed herein are methods of treating cancer. Some such methods include administration of a first anti-cancer agent that increases CD46 expression on the surface of a cancer cell. Some embodiments include administration of a second anti-cancer agent that binds CD46. The second anti-cancer agent may include an immunoconjugate comprising a CD46 binding domain and effector agent.

### CROSS-REFERENCE

This application claims the benefit of U.S. Provisional Application No. 63/134,896, filed Jan. 7, 2021, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Jan. 6, 2022, is named 39442-710_601_SL.txt and is 135,683 bytes in size.

### BACKGROUND

Cancer is a widespread problem. New therapies and treatment regimens are needed.

### SUMMARY

The present disclosure provides methods of treating cancer that include administration of a first anti-cancer agent, and a second anti-cancer agent. The first and second anti-cancer agents may work synergistically against the cancer.

Disclosed herein, in some embodiments, are methods of treating cancer in a subject in need thereof, said method comprising: administering to said subject a first anti-cancer agent that increases CD46 expression on the surface of a cancer cell; and administering to said subject a second anti-cancer agent comprising an antibody that specifically binds CD46 or a CD46-binding fragment thereof. In some embodiments, said first anti-cancer agent comprises a drug or prodrug thereof, a first antibody, a peptide, a protein, a liposome containing the drug or prodrug thereof, a radionuclide, a viral particle, or a chelate. In some embodiments, said first anti-cancer agent comprises a drug. In some embodiments, said drug comprises an anti-cancer drug, a chemotherapeutic agent, a microtubule inhibitor, a DNA-damaging agent, or a polymerase inhibitor. In some embodiments, said first anti-cancer agent comprises an immunotherapy. In some embodiments, the first anti-cancer agent is not pomalidomide, lenalidomide, or enzalutamide. In some embodiments, the first anti-cancer agent is not a pomalidomide analog, a lenalidomide analog, or an enzalutamide analog. In some embodiments, said second anti-cancer agent binds CD46 expressed on the surface of the cancer cell and is internalized into said cancer cell. In some embodiments, said second anti-cancer agent is internalized into the cancer cell via macropinocytosis. In some embodiments, said second anti-cancer agent comprises a constant region of an IgG heavy chain. In some embodiments, second anti-cancer agent comprises a constant region of an IgG1 heavy chain. In some embodiments, second anti-cancer agent comprises a single chain variable fragment (scFv), a single domain antibody (sdA), a Fab, or a Fab′. In some embodiments, the antibody that specifically binds CD46 or the CD46-binding fragment thereof binds domain 1 or 2 of CD46. In some embodiments, said second anti-cancer agent comprises one or more complementarity determining region (CDR) sequences comprising an amino acid sequence selected from SEQ ID NOs: 1-126. In some embodiments, the one or more CDR sequences comprise any of SEQ ID NOs: 1-3, 10-15, 64-66 or 73-78. In some embodiments, said second anti-cancer agent comprises a heavy chain (HC) variable region that comprises three CDRs: HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 1, SEQ ID NO: 2, and SEQ ID NO: 3, respectively, and said LC CDR1, LC CDR2, and LC CDR3 comprise an amino acid sequence of SEQ ID NO: 64, SEQ ID NO: 65, and SEQ ID NO: 66, respectively. In some embodiments, said second anti-cancer agent further comprises a cytotoxic effector coupled to said antibody that specifically binds CD46, or coupled to the CD46-binding fragment thereof. In some embodiments, said cytotoxic effector comprises a second drug or prodrug thereof, peptide, protein, liposome containing the drug or prodrug thereof, radionucleotide, viral particle, or chelate. In some embodiments, said cytotoxic effector comprises a second drug. In some embodiments, said second drug comprises an anti-cancer drug, a chemotherapeutic agent, a microtubule inhibitor, a DNA-damaging agent, or a polymerase inhibitor. In some embodiments, said second drug comprises auristatin, dolastatin-10, or maytansine, or a derivative thereof. In some embodiments, said second drug comprises monomethylauristatin F (MMAF), auristatin E (AE), monomethylauristatin E (MMAE), valine-citrulline MMAE (vcMMAE), or valine-citrulline MMAF (vcMMAF). In some embodiments, said second drug comprises monomethylauristatin E (MMAE). In some embodiments, a ratio of said cytotic effector to said antibody that specifically binds CD46 or a CD46-binding fragment thereof is about 2:1, 4:1, 6:1, or 8:1. In some embodiments, a ratio of said cytotic effector to said antibody that specifically binds CD46 or a CD46-binding fragment thereof is about 4:1. In some embodiments, said cytotoxic effector is conjugated to said antibody that specifically binds CD46 via a linker. In some embodiments, said linker comprises a peptide, small molecule, or a combination thereof In some embodiments, said linker comprises maleimidocaproyl-valine-citrulline-para-amino benzyloxycarbonyl (mc-vc-PAB). In some embodiments, said cancer comprises ovarian cancer, colorectal cancer, breast cancer, lung cancer, kidney cancer, pancreatic cancer, mesothelioma, lymphoma, liver cancer, urothelial cancer, stomach cancer, glioblastoma multiforme, glioma, neuroblastoma, or cervical cancer. In some embodiments, said cancer is not a prostate cancer or a multiple myeloma. In some embodiments, said subject is a mammal. In some embodiments, said subject is a human. In some embodiments, said first anti-cancer agent is administered as part of a first pharmaceutical composition. In some embodiments, said second anti-cancer agent is administered as part of said first pharmaceutical composition. In some embodiments, said second anti-cancer agent is administered as part of a second pharmaceutical composition. In some embodiments, said first and/or second pharmaceutical composition comprises from about 10 to 30 mM histidine buffer. In some embodiments, said first and/or second pharmaceutical composition comprises a cryoprotectant. In some embodiments, said cryoprotectant is a saccharide, sucrose, or trehalose. In some embodiments, said cryoprotectant is sucrose or trehalose. In some embodiments, said first and/or second pharmaceutical composition comprises a stabilizing agent. In some embodiments, said stabilizing agent prevents denaturation of said first anti-cancer agent, prevents aggregation of said second anti-cancer agent, or both. In some embodiments, said stabilizing agent comprises a polysorbate. In some embodiments, said stabilizing agent is polysorbate 80. In some embodiments, said first and/or second pharmaceutical composition comprises a pH from about 5.0 to 7.0. In some embodiments, said first anti-cancer agent and/or said second anti-cancer agent is administered to said human subject orally, nasally, rectally, intraperitoneally, subcutaneously, transcutaneously, intramuscularly, or intravenously. In some embodiments, said first anti-cancer agent and/or said second anti-cancer agent is administered to said human subject via intravenous infusion. In some embodiments, said first anti-cancer agent and/or second anti-cancer agent is administered in an effective amount. In some embodiments, said effective amount of the second anti-cancer agent comprises a dose from about 1.0 to 5.0 mg/kg. In some embodiments, said dose is about 1.2 mg/kg. In some embodiments, said dose is about 1.8 mg/kg. In some embodiments, said dose is about 2.4 mg/kg. In some embodiments, said dose is about 3.2 mg/kg. In some embodiments, said dose is administered every 2-4 weeks. In some embodiments, said dose is administered about every 3 weeks. In some embodiments, said effective amount of the first anti-cancer agent increases a response of the cancer cell to the second anti-cancer agent. In some embodiments, said increased CD46 expression on the surface of the cancer cell is relative to a control measurement or relative to a baseline measurement. In some embodiments, said first anti-cancer agent enhances an antibody-dependent cellular cytotoxicty activity of the second anti-cancer agent on the cancer cell. In some embodiments, said effective amount of the first anti-cancer agent or of the second anti-cancer agent is lower than an effective amount in a method not including administration of both the first anti-cancer agent and second anti-cancer agent. In some embodiments, a first dose of said first anti-cancer agent is administered before a first dose of said second anti-cancer agent.

Disclosed herein, in some embodiments, are methods of treating cancer in a subject comprising administering to the subject a first anti-cancer agent and administering to the subject a second anti-cancer agent that comprises an anti-CD46 antibody conjugated to a cytotoxic effector, wherein the combination of the first and second anti-cancer agents is synergistic in treating cancer in the subject, wherein the first anti-cancer agent is not pomalidomide, lenalidomide, or enzalutamide, and wherein the cancer is not prostate cancer or multiple myeloma. In some embodiments, the first anti-cancer agent is not a pomalidomide analog, a lenalidomide analog, or an enzalutamide analog. In some embodiments, said first anti-cancer agent comprises a drug or prodrug thereof, a first antibody, a peptide, a protein, a liposome containing the drug or prodrug thereof, a radionuclide, a viral particle, or a chelate. In some embodiments, said first anti-cancer agent comprises a drug. In some embodiments, said drug comprises an anti-cancer drug, a chemotherapeutic agent, a microtubule inhibitor, a DNA-damaging agent, or a polymerase inhibitor. In some embodiments, said first anti-cancer agent comprises an immunotherapy. In some embodiments, said second anti-cancer agent binds CD46 expressed on the cancer cells and is internalized into said cancer cells. In some embodiments, said second anti-cancer agent is internalized into the cancer cells via macropinocytosis. In some embodiments, said anti-CD46 antibody comprises a constant region of an IgG heavy chain. In some embodiments, said anti-CD46 antibody comprises a constant region of an IgG1 heavy chain. In some embodiments, the anti-CD46 antibody binds domain 1 or 2 of CD46. In some embodiments, said anti-CD46 antibody comprises one or more complementarity determining region (CDR) sequences one or more CDR sequences selected from SEQ ID NOs: 1-126. In some embodiments, the one or more CDR sequences comprise any of SEQ ID NOs: 1-3, 10-15, 64-66 or 73-78. In some embodiments, said anti-CD46 antibody comprises a heavy chain (HC) variable region that comprises three CDRs: HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 1, SEQ ID NO: 2, and SEQ ID NO: 3, respectively, and said LC CDR1, LC CDR2, and LC CDR3 comprise an amino acid sequence of SEQ ID NO: 64, SEQ ID NO: 65, and SEQ ID NO: 66, respectively. In some embodiments, said cytotoxic effector comprises a second drug or prodrug thereof, peptide, protein, liposome containing the drug or prodrug thereof, radionucleotide, viral particle, or chelate. In some embodiments, said cytotoxic effector comprises a second drug. In some embodiments, said second drug comprises an anti-cancer drug, a chemotherapeutic agent, a microtubule inhibitor, a DNA-damaging agent, or a polymerase inhibitor. In some embodiments, said second drug comprises auristatin, dolastatin-10, or maytansine, or a derivative thereof. In some embodiments, said second drug comprises monomethylauristatin F (MMAF), auristatin E (AE), monomethylauristatin E (MMAE), valine-citrulline MMAE (vcMMAE), or valine-citrulline MMAF (vcMMAF). In some embodiments, said second drug comprises monomethylauristatin E (MMAE). In some embodiments, a ratio of said cytotic effector to said anti-CD46 antibody is about 2:1, 4:1, 6:1, or 8:1. In some embodiments, a ratio of said cytotic effector to said anti-CD46 antibody is about 4:1. In some embodiments, said cytotoxic effector is conjugated to said anti-CD46 antibody via a linker. In some embodiments, said linker comprises a peptide, small molecule, or a combination thereof In some embodiments, said linker comprises maleimidocaproyl-valine-citrulline-para-amino benzyloxycarbonyl (mc-vc-PAB). In some embodiments, said cancer comprises ovarian cancer, colorectal cancer, breast cancer, lung cancer, kidney cancer, pancreatic cancer, mesothelioma, lymphoma, liver cancer, urothelial cancer, stomach cancer, glioblastoma multiforme, glioma, neuroblastoma, or cervical cancer. In some embodiments, said subject is a mammal. In some embodiments, said subject is a human. In some embodiments, said first anti-cancer agent is administered as part of a first pharmaceutical composition. In some embodiments, said second anti-cancer agent is administered as part of said first pharmaceutical composition. In some embodiments, said second anti-cancer agent is administered as part of a second pharmaceutical composition. In some embodiments, said first and/or second pharmaceutical composition comprises from about 10 to 30 mM histidine buffer. In some embodiments, said first and/or second pharmaceutical composition comprises a cryoprotectant. In some embodiments, said cryoprotectant is a saccharide, sucrose, or trehalose. In some embodiments, said ryoprotectant is sucrose or trehalose. In some embodiments, said first and/or second pharmaceutical composition comprises a stabilizing agent. In some embodiments, said stabilizing agent prevents denaturation of said first anti-cancer agent, prevents aggregation of said second anti-cancer agent, or both. In some embodiments, said stabilizing agent comprises a polysorbate. In some embodiments, said stabilizing agent is polysorbate 80. In some embodiments, said first and/or second pharmaceutical composition comprises a pH from about 5.0 to 7.0. In some embodiments, said first anti-cancer agent and/or said second anti-cancer agent is administered to said human subject orally, nasally, rectally, intraperitoneally, subcutaneously, transcutaneously, intramuscularly, or intravenously. In some embodiments, said first anti-cancer agent and/or said second anti-cancer agent is administered to said human subject via intravenous infusion. In some embodiments, said first anti-cancer agent and/or second anti-cancer agent is administered in an effective amount. In some embodiments, said effective amount of the second anti-cancer agent comprises a dose from about 1.0 to 5.0 mg/kg. In some embodiments, said dose is about 1.2 mg/kg. In some embodiments, said dose is about 1.8 mg/kg. In some embodiments, said dose is about 2.4 mg/kg. In some embodiments, said dose is about 3.2 mg/kg. In some embodiments, said dose is administered every 2-4 weeks. In some embodiments, said dose is administered about every 3 weeks. In some embodiments, said effective amount of the first anti-cancer agent increases a response of the cancer cells to the second anti-cancer agent. In some embodiments, said increased CD46 expression on the cancer cells is relative to a control measurement or relative to a baseline measurement. In some embodiments, said first anti-cancer agent enhances an antibody-dependent cellular cytotoxicty activity of the second anti-cancer agent on the cancer cells. In some embodiments, said effective amount of the first anti-cancer agent or of the second anti-cancer agent is lower than an effective amount in a method not including administration of both the first anti-cancer agent and second anti-cancer agent. In some embodiments, a first dose of said first anti-cancer agent is administered before a first dose of said second anti-cancer agent.

### BRIEF DESCRIPTION OF THE FIGURES

FIG. 1 shows results of an immunohistochemistry study of anti-CD46 antibodies on an FDA standard panel of frozen tissues for therapeutic antibody evaluation.

FIG. 2 is a diagram showing the structure of the FOR46 immunoconjugate described herein.

FIG. 3 is a box and whiskers plot comparing CD46 expression in paired breast cancer biopsies collected before and after treatment with anastrozole.

FIG. 4 is a box and whiskers plot comparing CD46 expression in paired esophageal cancer biopsies collected before and after treatment with fluorouracil.

FIG. 5 is a box and whiskers plot comparing CD46 expression in unpaired colorectal cancer biopsies collected before and after treatment with irinotecan.

FIG. 6 is a box and whiskers plot comparing CD46 expression in unpaired breast cancer biopsies collected before and after treatment with zoledronic acid.

FIG. 7 is a box and whiskers plot comparing CD46 expression in unpaired head and neck squamous cell carcinoma biopsies collected before and after treatment with cisplatin

FIG. 8 is a box and whiskers plot comparing CD46 expression in unpaired clear cell renal cell carcinoma biopsies collected before and after treatment with axitinib.

### DETAILED DESCRIPTION

CD46, also known as CD46 complement regulatory protein, cluster of differentiation 46 and membrane cofactor protein, is an inhibitory complement receptor. Overexpression of CD46 has been observed in several cancers, such as breast cancer, colorectal cancer, liver cancer, lung cancer, or prostate cancer. In some cases, overexpression of CD46 has been characterized as a negative prognostic factor. For example, overexpression of CD46 has been correlated with shorter progression-free time and shorter overall survival time in breast cancer patients and ovarian cancer patients. In some cases, CD46 is not overexpressed, but CD46 overexpression could be induced. New therapies and treatment regimens for the treatment of cancer are needed. Provided herein are anti-cancer agents that increase CD46 expression. Further provided herein are antibodies and immunoconjugates targeting CD46 for the treatment of cancer.

The present disclosure includes methods of treating cancer that include administration of a first anti-cancer agent, and a second anti-cancer agent. The first and second anti-cancer agents may work synergistically against the cancer. The first anti-cancer agent may increase CD46 on a cancer cell. The second anti-cancer agent may bind CD46.

Disclosed herein, in some embodiments, are methods of treating cancer in a subject in need thereof. Some embodiments include administering to said subject a first anti-cancer agent. In some embodiments, the first anti-cancer agent increases CD46 expression. In some embodiments, the CD46 expression is increased on a cancer cell. In some embodiments, the CD46 expression is increased on the surface of the cancer cell. Some embodiments include administering to said subject a second anti-cancer agent. In some embodiments, the second anti-cancer agent comprises an antibody that specifically binds CD46 or a CD46-binding fragment thereof. In some embodiments, the combination of the first and second anti-cancer agents is synergistic in treating cancer in the subject. In some embodiments, the first anti-cancer agent is not pomalidomide, lenalidomide or enzalutamide. In some embodiments, the cancer is not prostate cancer or multiple myeloma. Disclosed herein, in some embodiments, are methods of treating cancer in a subject in need thereof, said method comprising: administering to said subject a first anti-cancer agent that increases CD46 expression on the surface of a cancer cell; and administering to said subject a second anti-cancer agent comprising an antibody that specifically binds CD46 or a CD46-binding fragment thereof

Disclosed herein, in some embodiments, are methods of treating cancer in a subject. Some embodiments include administering to the subject a first anti-cancer agent. In some embodiments, the first anti-cancer agent increases the expression of CD46 on cancer cells in the subject. Some embodiments include administering to the subject a second anti-cancer agent. In some embodiments, the second anti-cancer agent comprises an anti-CD46 antibody. In some embodiments, the anti-CD46 antibody is conjugated to a cytotoxic effector. In some embodiments, the combination of the first and second anti-cancer agents is synergistic in treating cancer in the subject. In some embodiments, the first anti-cancer agent is not pomalidomide or lenalidomide, or is not another drug described herein. In some embodiments, the cancer is not prostate cancer or multiple myeloma. Disclosed herein, in some embodiments, are methods of treating cancer in a subject comprising administering to the subject a first anti-cancer agent that increases the expression of CD46 on cancer cells in the subject and administering to the subject a second anti-cancer agent that comprises an anti-CD46 antibody conjugated to a cytotoxic effector, wherein the combination of the first and second anti-cancer agents is synergistic in treating cancer in the subject, wherein the first anti-cancer agent is not pomalidomide, lenalidomide, or enzalutamide, and wherein the cancer is not prostate cancer or multiple myeloma.

### Anti-Cancer Agents that Increase CD46 Expression

In some embodiments, disclosed herein is an anti-cancer agent. The anti-cancer agent may increase CD46 expression. The anti-cancer agent may be used in the methods of treating cancer described herein. For example, the anti-cancer agent may be administered to a subject in need thereof as a first anti-cancer agent.

In some embodiments, said first anti-cancer agent comprises a drug or prodrug thereof, a first antibody, a peptide, a protein, a liposome containing the drug or prodrug thereof, a radionuclide, a viral particle, or a chelate. In some embodiments, said first anti-cancer agent comprises a drug. In some embodiments, said drug comprises an anti-cancer drug, a chemotherapeutic agent, a microtubule inhibitor, a DNA-damaging agent, or a polymerase inhibitor. In some embodiments, said first anti-cancer agent comprises an immunotherapy.

Examples of anti-cancer agents include Abemaciclib, Abiraterone Acetate, Abraxane (Paclitaxel Albumin-stabilized Nanoparticle Formulation), ABVD, ABVE, ABVE-PC, AC, Acalabrutinib, AC-T, Actemra (Tocilizumab), Adcetris (Brentuximab Vedotin), ADE, Ado-Trastuzumab Emtansine, Adriamycin (Doxorubicin Hydrochloride), Afatinib Dimaleate, Afinitor (Everolimus), Akynzeo (Netupitant and Palonosetron Hydrochloride), Aldara (Imiquimod), Aldesleukin, Alecensa (Alectinib), Alectinib, Alemtuzumab, Alimta (Pemetrexed Disodium), Aliqopa (Copanlisib Hydrochloride), Alkeran for Injection (Melphalan Hydrochloride), Alkeran Tablets (Melphalan), Aloxi (Palonosetron Hydrochloride), Alpelisib, Alunbrig (Brigatinib), Ameluz (Aminolevulinic Acid Hydrochloride), Amifostine, Aminolevulinic Acid Hydrochloride, Anastrozole, Apalutamide, Aprepitant, Aranesp (Darbepoetin Alfa), Aredia (Pamidronate Disodium), Arimidex (Anastrozole), Aromasin (Exemestane), Arranon (Nelarabine), Arsenic Trioxide, Arzerra (Ofatumumab), Asparaginase Erwinia chrysanthemi, Asparlas (Calaspargase Pegol-mknl), Atezolizumab, Avapritinib, Avastin (Bevacizumab), Avelumab, Axicabtagene Ciloleucel, Axitinib, Ayvakit (Avapritinib), Azacitidine, Azedra (Iobenguane I 131), Balversa (Erdafitinib), Bavencio (Avelumab), BEACOPP, Belantamab Mafodotin-blmf, Beleodaq (Belinostat), Belinostat, Bendamustine Hydrochloride, Bendeka (Bendamustine Hydrochloride), BEP, Besponsa (Inotuzumab Ozogamicin), Bevacizumab, Bexarotene, Bicalutami de, BiCNU (Carmustine), Binimetinib, Blenrep (Belantamab Mafodotin-blmf), Bleomycin Sulfate, Blinatumomab, Blincyto (Blinatumomab), Bortezomib, Bosulif (Bosutinib), Bosutinib, Braftovi (Encorafenib), Brentuximab Vedotin, Brexucabtagene Autoleucel, Brigatinib, Brukinsa (Zanubrutinib), BuMel, Busulfan, Busulfex (Busulfan), Cabazitaxel, Cablivi (Caplacizumab-yhdp), Cabometyx (Cabozantinib-S-Malate), Cabozantinib-S-Malate, CAF, Calaspargase Pegol-mknl, Calquence (Acalabrutinib), Campath (Alemtuzumab), Camptosar (Irinotecan Hydrochloride), Capecitabine, Caplacizumab-yhdp, Capmatinib Hydrochloride, CAPDX, Carac (Fluorouracil-Topical), Carboplatin, CARBOPLATIN-TAXOL, Carfilzomib, Carmustine, Carmustine Implant, Casodex (Bicalutamide), CEM, Cemiplimab-rwlc, Ceritinib, Cerubidine (Daunorubicin Hydrochloride), Cervarix (Recombinant HPV Bivalent Vaccine), Cetuximab, CEV, Chlorambucil, CHLORAMBUCIL-PREDNISONE, CHOP, Cisplatin, Cladribine, Clofarabine, Clolar (Clofarabine), CMF, Cobimetinib Fumarate, Cometriq (Cabozantinib-S-Malate), Copanlisib Hydrochloride, COPDAC, Copiktra (Duvelisib), COPP, COPP-ABV, Cosmegen (Dactinomycin), Cotellic (Cobimetinib Fumarate), Crizotinib, CVP, Cyclophosphamide, Cyramza (Ramucirumab), Cytarabine, Dabrafenib Mesylate, Dacarbazine, Dacogen (Decitabine), Dacomitinib, Dactinomycin, Daratumumab, Daratumumab and Hyaluronidase-fihj, Darbepoetin Alfa, Darolutamide, Darzalex (Daratumumab), Darzalex Faspro (Daratumumab and Hyaluronidase-fihj), Dasatinib, Daunorubicin Hydrochloride, Daunorubicin Hydrochloride and Cytarabine Liposome, Daurismo (Glasdegib Maleate), Decitabine, Decitabine and Cedazuridine, Defibrotide Sodium, Defitelio (Defibrotide Sodium), Degarelix, Denileukin Diftitox, Denosumab, Dexamethasone, Dexrazoxane Hydrochloride, Dinutuximab, Docetaxel, Doxil (Doxorubicin Hydrochloride Liposome), Doxorubicin Hydrochloride, Doxorubicin Hydrochloride Liposome, Durvalumab, Duvelisib, Efudex (Fluorouracil-Topical), Eligard (Leuprolide Acetate), Elitek (Rasburicase), Ellence (Epirubicin Hydrochloride), Elotuzumab, Eloxatin (Oxaliplatin), Eltrombopag Olamine, Elzonris (Tagraxofusp-erzs), Emapalumab-lzsg, Emend (Aprepitant), Empliciti (Elotuzumab), Enasidenib Mesylate, Encorafenib, Enfortumab Vedotin-ejfv, Enhertu (Fam-Trastuzumab Deruxtecan-nxki), Entrectinib, Enzalutamide, Epirubicin Hydrochloride, EPOCH, Epoetin Alfa, Epogen (Epoetin Alfa), Erbitux (Cetuximab), Erdafitinib, Eribulin Mesylate, Erivedge (Vismodegib), Erleada (Apalutamide), Erlotinib Hydrochloride, Erwinaze (Asparaginase Erwinia chrysanthemi), Ethyol (Amifostine), Etopophos (Etoposide Phosphate), Etoposide, Etoposide Phosphate, Everolimus, Evista (Raloxifene Hydrochloride), Evomela (Melphalan Hydrochloride), Exemestane, 5-FU (Fluorouracil Injection), 5-FU (Fluorouracil-Topical), Fam-Trastuzumab Deruxtecan-nxki, Fareston (Toremifene), Farydak (Panobinostat), Faslodex (Fulvestrant), FEC, Fedratinib Hydrochloride, Femara (Letrozole), Filgrastim, Firmagon (Degarelix), Fludarabine Phosphate, Fluoroplex (Fluorouracil-Topical), Fluorouracil Injection, Fluorouracil-Topical, Flutamide, FOLFIRI, FOLFIRI-BEVACIZUMAB, FOLFIRI-CETUXIMAB, FOLFIRINOX, FOLFOX, Folotyn (Pralatrexate), Fostamatinib Disodium, Fulphila (Pegfilgrastim), FU-LV, Fulvestrant, Gamifant (Emapalumab-lzsg), Gardasil (Recombinant HPV Quadrivalent Vaccine), Gardasil 9 (Recombinant HPV Nonavalent Vaccine), Gavreto (Pralsetinib), Gazyva (Obinutuzumab), Gefitinib, Gemcitabine Hydrochloride, GEMCITABINE-CISPLATIN, GEMCITABINE-OXALIPLATIN, Gemtuzumab Ozogamicin, Gemzar (Gemcitabine Hydrochloride), Gilotrif (Afatinib Dimaleate), Gilteritinib Fumarate, Glasdegib Maleate, Gleevec (Imatinib Mesylate), Gliadel Wafer (Carmustine Implant), Glucarpidase, Goserelin Acetate, Granisetron, Granisetron Hydrochloride, Granix (Filgrastim), Halaven (Eribulin Mesylate), Hemangeol (Propranolol Hydrochloride), Herceptin Hylecta (Trastuzumab and Hyaluronidase-oysk), Herceptin (Trastuzumab), HPV Bivalent Vaccine, Recombinant, HPV Nonavalent Vaccine, Recombinant, HPV Quadrivalent Vaccine, Recombinant, Hycamtin (Topotecan Hydrochloride), Hydrea (Hydroxyurea), Hydroxyurea, Hyper-CVAD, Ibrance (Palbociclib), Ibritumomab Tiuxetan, Ibrutinib, ICE, Iclusig (Ponatinib Hydrochloride), Idamycin PFS (Idarubicin Hydrochloride), Idarubicin Hydrochloride, Idelali sib, Idhifa (Enasidenib Mesylate), Ifex (Ifosfamide), Ifosfamide, IL-2 (Aldesleukin), Imatinib Mesylate, Imbruvica (Ibrutinib), Imfinzi (Durvalumab), Imiquimod, Imlygic (Talimogene Laherparepvec), Infugem (Gemcitabine Hydrochloride), Inlyta (Axitinib), Inotuzumab Ozogamicin, Inqovi (Decitabine and Cedazuridine), Inrebic (Fedratinib Hydrochloride), Interferon Alfa-2b, Recombinant, Interleukin-2 (Aldesleukin), Intron A (Recombinant Interferon Alfa-2b), Iobenguane I 131, Ipilimumab, Iressa (Gefitinib), Irinotecan Hydrochloride, Irinotecan Hydrochloride Liposome, Isatuximab-irfc, Istodax (Romidepsin), Ivosidenib, Ixabepilone, Ixazomib Citrate, Ixempra (Ixabepilone), Jakafi (Ruxolitinib Phosphate), JEB, Jelmyto (Mitomycin), Jevtana (Cabazitaxel), Kadcyla (Ado-Trastuzumab Emtansine), Kepivance (Palifermin), Keytruda (Pembrolizumab), Kisqali (Ribociclib), Koselugo (Selumetinib Sulfate), Kymriah (Tisagenlecleucel), Kyprolis (Carfilzomib), Lanreotide Acetate, Lapatinib Ditosylate, Larotrectinib Sulfate, Lenvatinib Mesylate, Lenvima (Lenvatinib Mesylate), Letrozole, Leucovorin Calcium, Leukeran (Chlorambucil), Leuprolide Acetate, Levulan Kerastik (Aminolevulinic Acid Hydrochloride), Libtayo (Cemiplimab-rwlc), Lomustine, Lonsurf (Trifluridine and Tipiracil Hydrochloride), Lorbrena (Lorlatinib), Lorlatinib, Lumoxiti (Moxetumomab Pasudotox-tdfk), Lupron Depot (Leuprolide Acetate), Lurbinectedin, Luspatercept-aamt, Lutathera (Lutetium Lu 177-Dotatate), Lutetium (Lu 177-Dotatate), Lynparza (Olaparib), Marqibo (Vincristine Sulfate Liposome), Matulane (Procarbazine Hydrochloride), Mechlorethamine Hydrochloride, Megestrol Acetate, Mekinist (Trametinib), Mektovi (Binimetinib), Melphalan, Melphalan Hydrochloride, Mercaptopurine, Mesna, Mesnex (Mesna), Methotrexate Sodium, Methylnaltrexone Bromide, Midostaurin, Mitomycin , Mitoxantrone Hydrochloride, Mogamulizumab-kpkc, Monjuvi (Tafasitamab-cxix), Moxetumomab Pasudotox-tdfk, Mozobil (Plerixafor), MVAC, Mvasi (Bevacizumab), Myleran (Busulfan), Mylotarg (Gemtuzumab Ozogamicin), Nanoparticle Paclitaxel (Paclitaxel Albumin-stabilized Nanoparticle Formulation), Necitumumab, Nelarabine, Neratinib Maleate, Nerlynx (Neratinib Maleate), Netupitant and Palonosetron Hydrochloride, Neulasta (Pegfilgrastim), Neupogen (Filgrastim), Nexavar (Sorafenib Tosylate), Nilandron (Nilutamide), Nilotinib, Nilutamide, Ninlaro (Ixazomib Citrate), Niraparib Tosylate Monohydrate, Nivolumab, Nplate (Romiplostim), Nubeqa (Darolutamide), Nyvepria (Pegfilgrastim), Obinutuzumab, Odomzo (Sonidegib), OEPA, Ofatumumab, OFF, Olaparib, Omacetaxine Mepesuccinate, Oncaspar (Pegaspargase), Ondansetron Hydrochloride, Onivyde (Irinotecan Hydrochloride Liposome), Ontak (Denileukin Diftitox), Onureg (Azacitidine), Opdivo (Nivolumab), OPPA, Osimertinib Mesylate, Oxaliplatin, Paclitaxel, Paclitaxel Albumin-stabilized Nanoparticle Formulation, PAD, Padcev (Enfortumab Vedotin-ejfv), Palbociclib, Palifermin, Palonosetron Hydrochloride, Palonosetron Hydrochloride and Netupitant, Pamidronate Disodium, Panitumumab, Panobinostat, Pazopanib Hydrochloride, PCV, PEB, Pegaspargase, Pegfilgrastim, Peginterferon Alfa-2b, PEG-Intron (Peginterferon Alfa-2b), Pemazyre (Pemigatinib), Pembrolizumab, Pemetrexed Disodium, Pemigatinib, Perj eta (Pertuzumab), Pertuzumab, Pertuzumab, Trastuzumab, and Hyaluronidase-zzxf, Pexidartinib Hydrochloride, Phesgo (Pertuzumab, Trastuzumab, and Hyaluronidase-zzxf), Piqray (Alpelisib), Plerixafor, Polatuzumab Vedotin-piiq, Polivy (Polatuzumab Vedotin-piiq), Ponatinib Hydrochloride, Portrazza (Necitumumab), Poteligeo (Mogamulizumab-kpkc), Pralatrexate, Pralsetinib, Prednisone, Procarbazine Hydrochloride, Procrit (Epoetin Alfa), Proleukin (Aldesleukin), Prolia (Denosumab), Promacta (Eltrombopag Olamine), Propranolol Hydrochloride, Provenge (Sipuleucel-T), Purinethol (Mercaptopurine), Purixan (Mercaptopurine), Qinlock (Ripretinib), Radium 223 Dichloride, Raloxifene Hydrochloride, Ramucirumab, Rasburicase, Ravulizumab-cwvz, Reblozyl (Luspatercept-aamt), R-CHOP, R-CVP, Recombinant Human Papillomavirus (HPV) Bivalent Vaccine, Recombinant Human Papillomavirus (HPV) Nonavalent Vaccine, Recombinant Human Papillomavirus (HPV) Quadrivalent Vaccine, Recombinant Interferon Alfa-2b, Regorafenib, Relistor (Methylnaltrexone Bromide), R-EPOCH, Retacrit (Epoetin Alfa), Retevmo (Selpercatinib), Ribociclib, R-ICE, Ripretinib, Rituxan (Rituximab), Rituxan Hycela (Rituximab and Hyaluronidase Human), Rituximab, Rituximab and Hyaluronidase Human, Rolapitant Hydrochloride, Romidepsin, Romiplostim, Rozlytrek (Entrectinib), Rubidomycin (Daunorubicin Hydrochloride), Rubraca (Rucaparib Camsylate), Rucaparib Camsylate, Ruxolitinib Phosphate, Rydapt (Midostaurin), Sacituzumab Govitecan-hziy, Sancuso (Granisetron), Sarclisa (Isatuximab-irfc), Sclerosol Intrapleural Aerosol (Talc), Selinexor, Selpercatinib, Selumetinib Sulfate, Siltuximab, Sipuleucel-T, Somatuline Depot (Lanreotide Acetate), Sonidegib, Sorafenib Tosylate, Sprycel (Dasatinib), STANFORD V, Sterile Talc Powder (Talc), Steritalc (Talc), Stivarga (Regorafenib), Sunitinib Malate, Sustol (Granisetron), Sutent (Sunitinib Malate), Sylatron (Peginterferon Alfa-2b), Sylvant (Siltuximab), Synribo (Omacetaxine Mepesuccinate), Tabloid (Thioguanine), Tabrecta (Capmatinib Hydrochloride), TAC, Tafasitamab-cxix, Tafinlar (Dabrafenib Mesylate), Tagraxofusp-erzs, Tagrisso (Osimertinib Mesylate), Talazoparib Tosyl ate, Talc, Talimogene Laherparepvec, Talzenna (Talazoparib Tosylate), Tamoxifen Citrate, Tarceva (Erlotinib Hydrochloride), Targretin (Bexarotene), Tasigna (Nilotinib), Tavalisse (Fostamatinib Di sodium), Taxotere (Docetaxel), Tazemetostat Hydrobromide, Tazverik (Tazemetostat Hydrobromide), Tecartus (Brexucabtagene Autoleucel), Tecentriq (Atezolizumab), Temodar (Temozolomide), Temozolomi de, Temsirolimus, Thioguanine, Thiotepa, Tibsovo (Ivosidenib), Ti sagenlecleucel, Tocilizumab, Tolak (Fluorouracil-Topical), Topotecan Hydrochloride, Toremifene, Torisel (Temsirolimus), Totect (Dexrazoxane Hydrochloride), TPF, Trabectedin, Trametinib, Trastuzumab, Trastuzumab and Hyaluronidase-oysk, Treanda (Bendamustine Hydrochloride), Trexall (Methotrexate Sodium), Trifluridine and Tipiracil Hydrochloride, Trisenox (Arsenic Trioxide), Trodelvy (Sacituzumab Govitecan-hziy), Truxima (Rituximab), Tucatinib, Tukysa (Tucatinib), Turalio (Pexidartinib Hydrochloride), Tykerb (Lapatinib Ditosylate), Ultomiris (Ravulizumab-cwvz), Undencyca (Pegfilgrastim), Unituxin (Dinutuximab), Uridine Triacetate, VAC, Valrubicin, Val star (Valrubicin), Vandetanib, VAMP, Varubi (Rolapitant Hydrochloride), Vectibix (Panitumumab), VeIP, Velcade (Bortezomib), Vemurafenib, Venclexta (Venetoclax), Venetoclax, Verzenio (Abemaciclib), Vidaza (Azacitidine), Vinblastine Sulfate, Vincristine Sulfate, Vincristine Sulfate Liposome, Vinorelbine Tartrate, VIP, Vismodegib, Vistogard (Uridine Triacetate), Vitrakvi (Larotrectinib Sulfate), Vizimpro (Dacomitinib), Voraxaze (Glucarpidase), Vorinostat, Votrient (Pazopanib Hydrochloride), Vyxeos (Daunorubicin Hydrochloride and Cytarabine Liposome), Xalkori (Crizotinib), Xatmep (Methotrexate Sodium), Xeloda (Capecitabine), XELIRI, XELOX, Xgeva (Denosumab), Xofigo (Radium 223 Dichloride), Xospata (Gilteritinib Fumarate), Xpovio (Selinexor), Xtandi (Enzalutamide), Yervoy (Ipilimumab), Yescarta (Axicabtagene Ciloleucel), Yondelis (Trabectedin), Yonsa (Abiraterone Acetate), Zaltrap (Ziv-Aflibercept), Zanubrutinib , Zarxio (Filgrastim), Zejula (Niraparib Tosylate Monohydrate), Zelboraf (Vemurafenib), Zepzelca (Lurbinectedin), Zevalin (Ibritumomab Tiuxetan), Ziextenzo (Pegfilgrastim), Zinecard (Dexrazoxane Hydrochloride), Zirabev (Bevcizumab), Ziv-Aflibercept, Zofran (Ondansetron Hydrochloride), Zoladex (Goserelin Acetate), Zoledronic Acid, Zolinza (Vorinostat), Zometa (Zoledronic Acid), Zyclara (Imiquimod), Zydelig (Idelalisib), Zykadia (Ceritinib), or Zytiga (Abiraterone Acetate). Each of these may be tested to determine if whether they increase CD46, and may then be included as the first anti-cancer agent if they do. Any of these anti-cancer agents may also be included in a second anti-cancer agent, conjugated to an anti-CD46 antibody or binding fragment.

In some embodiments, the first anti-cancer agent is not pomalidomide, lenalidomide, or enzalutamide. In some embodiments, the first anti-cancer agent is not pomalidomide. In some embodiments, the first anti-cancer agent is not lenalidomide. In some embodiments, the first anti-cancer agent is not enzalutamide. In some embodiments, the first anti-cancer agent is not a pomalidomide analog, a lenalidomide analog, or an enzalutamide analog. In some embodiments, the first anti-cancer agent is not a pomalidomide analog. In some embodiments, the first anti-cancer agent is not a lenalidomide analog. In some embodiments, the first anti-cancer agent is not an enzalutamide analog.

### Anti-CD46 Antibodies and Antibody Fragments

In some embodiments, disclosed herein is an antibody (or antigen binding fragment thereof) that specifically binds CD46 (also referred to herein as an anti-CD46 antibody). The disclosure includes anti-CD46 recombinant antibodies, but may include anti-CD46 antibodies wherever anti-CD46 recombinant antibodies are described. The anti-CD46 antibodies may be used in the methods of treating cancer described herein. For example, the anti-CD46 antibody may be included in a second anti-cancer agent.

In some embodiments, said second anti-cancer agent binds CD46 expressed on the surface of the cancer cell and is internalized into said cancer cell. In some embodiments, said second anti-cancer agent is internalized into the cancer cell via macropinocytosis.

In some embodiments, an antibody or antigen binding fragment or variant thereof is a monoclonal antibody. In some embodiments, an antibody or antigen binding fragment or variant thereof is a human antibody, a murine antibody, a humanized antibody, or a chimeric antibody. In some embodiments, the antibody comprises or consists of a function fragment of a full length antibody (e.g., an antigen binding fragment of a full length antibody) such as a monovalent Fab, a bivalent Fab′2, a single-chain variable fragment (scFv), or functional fragment or variant thereof In some embodiments, the recombinant antibody (or antigen binding fragment thereof) comprises an immunoglobulin variable heavy chain domain (VH). In some embodiments, the recombinant antibody (or antigen binding fragment thereof) comprises an immunoglobulin variable light chain domain (VL). In some embodiments, the recombinant antibody (or antigen binding fragment thereof) comprises a VH and a VL. In some embodiments, said second anti-cancer agent comprises a scFv, a single domain antibody (sdA), a Fab, or a Fab′. In some embodiments, said second anti-cancer agent comprises a scFv. In some embodiments, said second anti-cancer agent comprises a sdA. In some embodiments, said second anti-cancer agent comprises a Fab. In some embodiments, said second anti-cancer agent comprises a Fab′.

In some embodiments, the recombinant antibody (or antigen binding fragment thereof) comprises an Fc region. In some embodiments, the recombinant antibody (or antigen binding fragment thereof) is a full length antibody. In some embodiments, the recombinant antibody (or antigen binding fragment thereof) comprises a first light chain that comprises a light chain variable region and a light chain constant region; a first heavy chain that comprises a heavy chain variable region and a heavy chain constant region; a second light chain that comprises a light chain variable region and a light chain constant region; and a second heavy chain that comprises a heavy chain variable region and a heavy chain constant region. In some embodiments, the first and second light chains have at least 95%, 96%, 97%, 98%, 99%, or 100% sequence identity. In some embodiments, the first and second light chains bind the same epitope. In some embodiments, the first and second heavy chains have at least 95%, 96%, 97%, 98%, 99%, or 100% sequence identity. In some embodiments, the first and second heavy chains bind the same epitope.

In some embodiments, the recombinant antibody (or antigen binding fragment thereof) is derived from non-human (e.g. rabbit or mouse) antibodies. In some instances, the humanized form of the non-human antibody contains a minimal non-human sequence to maintain original antigenic specificity. In some cases, the humanized antibodies are human immunoglobulins (acceptor antibody), wherein the CDRs of the acceptor antibody are replaced by residues of the CDRs of a non-human immunoglobulin (donor antibody), such as rat, rabbit, or mouse donor having the desired specificity, affinity, avidity, binding kinetics, and/or capacity. In some instances, one or more framework region (FR) residues of the human immunoglobulin are replaced by corresponding non-human residues of the donor antibody.

In some embodiments, the antibody that specifically binds CD46 or the CD46-binding fragment thereof binds domain 1 or 2 of CD46. In some embodiments, the antibody that specifically binds CD46 or the CD46-binding fragment thereof binds domain 1 of CD46. In some embodiments, the antibody that specifically binds CD46 or the CD46-binding fragment thereof binds domain 2 of CD46. In some embodiments, the antibody that specifically binds CD46 or the CD46-binding fragment thereof does not bind domain 3 or 4 of CD46, or does not block a complement cascade. In some embodiments, the antibody that specifically binds CD46 or CD46-binding fragment thereof comprises an anti-CD46 antibody or binding fragment disclosed in WO 201640683 (PCT/US2015/049492) the contents of which are incorporated herein in their entirety. Additional anti-CD46 antibodies or binding fragments may be found in PCT/US2008/076704 (WO 2009/039192); U.S. Pat. No. 10/272,835 (US 2003/0108966); PCT/NL2001/000636 (WO 2002/018948); Sherbenou et al, Antibody-drug conjugate targeting CD46 eliminates multiple myeloma cells, J Clin Invest. 2016 Dec. 1;126(12):4640-4653; Su, Targeting CD46 for both adenocarcinoma and neuroendocrine prostate cancer, JCI Insight. 2018 Sep. 6;3(17):e121497; or Geuij en, Affinity ranking of antibodies using flow cytometry: application in antibody phage display-based target discovery, J Immunol Methods. 2005 July;302(1-2):68-77; all of which are incorporated herein by reference in their entireties.

### Complementarity Determining Regions (CDRs)

In some embodiments, the CD46 binding recombinant antibody comprises an immunoglobulin variable heavy chain domain (VH) that comprises at least one, two, or three complementarity determining regions (CDRs) disclosed in Table 1 or a sequence substantially identical thereto (e.g., a sequence that has at least 90%, 95%, 96%, 97%, 98%, or 99% sequence identity).

In some embodiments, the CD46 binding recombinant antibody comprises an immunoglobulin variable light chain domain (VL) that comprises at least one, two, or three complementarity determining regions (CDRs) disclosed in Table 2 or a sequence substantially identical thereto (e.g., a sequence that has at least 90%, 95%, 96%, 97%, 98%, or 99% sequence identity).

In some embodiments, the CD46 binding recombinant antibody comprises a VH that comprises at least one, two, or three complementarity determining regions (CDRs) disclosed in Table 1 or a sequence substantially identical thereto (e.g., a sequence that has at least 90%, 95%, 96%, 97%, 98%, or 99% sequence identity); and a VL that comprises at least one, two, or three complementarity determining regions (CDRs) disclosed in Table 2 or a sequence substantially identical thereto (e.g., a sequence that has at least 90%, 95%, 96%, 97%, 98%, or 99% sequence identity).

In some embodiments, the CD46 binding recombinant antibody comprises a VH that comprises a CDR1 of SEQ ID NO: 1, a CDR2 of SEQ ID NO: 2, and a CDR3 of SEQ ID NO: 3.

In some embodiments, the CD46 binding recombinant antibody comprises a VL that comprises a CDR1 of SEQ ID NO: 64, a CDR2 of SEQ ID NO: 65, and a CDR3 of SEQ ID NO: 66.

In some embodiments, the CD46 binding recombinant antibody comprises a VH that comprises a CDR1 of SEQ ID NO: 1, a CDR2 of SEQ ID NO: 2, and a CDR3 of SEQ ID NO: 3; and a VL that comprises a CDR1 of SEQ ID NO: 64, a CDR2 of SEQ ID NO: 65, and a CDR3 of SEQ ID NO: 66.

In some embodiments, the CD46 binding recombinant antibody comprises a VH that comprises a CDR1 of SEQ ID NO: 10, a CDR2 of SEQ ID NO: 11, and a CDR3 of SEQ ID NO: 12.

In some embodiments, the CD46 binding recombinant antibody comprises a VL that comprises a CDR1 of SEQ ID NO: 73, a CDR2 of SEQ ID NO: 74, and a CDR3 of SEQ ID NO: 75.

In some embodiments, the CD46 binding recombinant antibody comprises a VH that comprises a CDR1 of SEQ ID NO: 10, a CDR2 of SEQ ID NO: 11, and a CDR3 of SEQ ID NO: 12; and a VL that comprises a CDR1 of SEQ ID NO: 73, a CDR2 of SEQ ID NO: 74, and a CDR3 of SEQ ID NO: 75.

In some embodiments, the CD46 binding recombinant antibody comprises a VH that comprises a CDR1 of SEQ ID NO: 13, a CDR2 of SEQ ID NO: 14, and a CDR3 of SEQ ID NO: 15.

In some embodiments, the CD46 binding recombinant antibody comprises a VL that comprises a CDR1 of SEQ ID NO: 76, a CDR2 of SEQ ID NO: 77, and a CDR3 of SEQ ID NO: 78.

In some embodiments, the CD46 binding recombinant antibody comprises a VH that comprises a CDR1 of SEQ ID NO: 13, a CDR2 of SEQ ID NO: 14, and a CDR3 of SEQ ID NO: 15; and a VL that comprises a CDR1 of SEQ ID NO: 76, a CDR2 of SEQ ID NO: 77, and a CDR3 of SEQ ID NO: 78.

TABLE 1
VH CDR amino acid sequences of anti-CD46 antibodies

| | SEQ ID | | SEQ ID | | SEQ ID | |
|---|---|---|---|---|---|---|
| Antibody | NO | CDR1 | NO | CDR2 | NO | CDR3 |
| YS5FL | 1 | GLTVNNYA | 2 | ISYDGNNK | 3 | AKGGGYFDL |
| YS5F | 4 | GFTVNNYA | 5 | ISYDGNNK | 6 | AKGGGYFDL |
| YS5vID | 7 | GFTVNNYA | 8 | ISYDGNNK | 9 | AKGGGYFDL |
| SB1HGNY | 10 | GFTFSSYA | 11 | IRSDGSKK | 12 | ARHGNYFDS |
| YS12 | 13 | GFTFSTYG | 14 | ISYDGDEK | 15 | AKASGYGMGILDY |
| 3G7RY aka | 16 | GFTFSDYY | 17 | ISSSGSTI | 18 | ARDYGRIAAAGRRY |
| 3G8 | | | | | | |
| YS6 | 19 | GFTFSDYY | 20 | ISSSGSTI | 21 | ARDYGRIAAAGRHY |
| YS1 | 22 | GFTFSDYY | 23 | ISSSGSTI | 24 | ARDYGRIAAAGRHY |
| YS3 | 25 | GFTFSSYW | 26 | IKQDGSEK | 27 | AKDVGSTAINYVRAY |
| | | | | | | TWFDP |
| YS4 | 28 | GFTFSNYA | 29 | ISGSGSST | 30 | AQGLYSSGWANWFD |
| | | | | | | P |
| YS8 | 31 | GFTFSSYG | 32 | ISYDGSNK | 33 | AKVMGLAAAGLDAF |
| | | | | | | DI |
| YS7 | 34 | GFTFSSYA | 35 | ISYDGSNK | 36 | GRESSGSPGV |
| YS9 | 37 | GFTVSSNY | 38 | IYTDGST | 39 | ARDRGTSGYDWAWF |
| | | | | | | DL |
| YS10 | 40 | GFTFSSYA | 41 | ISGSGGST | 42 | AKDRYYYGSGKDAF |
| | | | | | | DI |
| YS11 | 43 | GFTFSNYW | 44 | VRQDGGQK | 45 | VSQRNSGEHDY |
| 3G7HY | 46 | GFTFSDYY | 47 | ISSSGSTI | 48 | ARDYGRIAAAGRHY |
| 3G7NY | 49 | GFTFSDYY | 50 | ISSSGSTI | 51 | ARDYGRIAAAGRNY |
| 3G7 | 52 | GFTFSDYY | 53 | ISSSGSTI | 54 | ARDYGRIAAAGRHY |
| SB2 | 55 | GFTFSDYY | 56 | ISSSGSSI | 57 | ARDITDVVGVSFDY |
| 2C8 | 58 | GFTFSSYG | 59 | ISYDGSNK | 60 | AKVMGLAAAGLDAF |
| | | | | | | DI |
| UA8kappa | 61 | GFTFSSFG | 62 | ISYDGSNQ | 63 | GSRPGGGYASGSTVA |
| | | | | | | Y |

TABLE 2
VL CDR amino acid sequences of anti-CD46 antibodies

| | SEQ ID | | SEQ ID | | SEQ ID | |
|---|---|---|---|---|---|---|
| Antibody | NO | CDR1 | NO | CDR2 | NO | CDR3 |
| YS5FL | 64 | SSNIGAGYD | 65 | GNN | 66 | SSYTSGTWL |
| YS5F | 67 | SSNIGAGYD | 68 | GNN | 69 | SSYTSGTWL |
| YS5vID | 70 | SSNIGAGYD | 71 | GDN | 72 | SSYTSGTWL |
| SB1HGNY | 73 | QGISSY | 74 | AAS | 75 | QQLASYPLT |
| YS12 | 76 | SLRSYY | 77 | G?N | 78 | HSRDSSGTHLRV |
| 3G7RY aka | 79 | TSNIGSNH | 80 | RNN | 81 | ATWDDSLSGEV |
| 3G8 | | | | | | |
| YS6 | 82 | SLRSYY | 83 | GKN | 84 | NSRDSSGTHLEV |
| YS1 | 85 | TLSTYY | 86 | GKN | 87 | HSRDISGNYL |
| YS3 | 88 | SSNIGSNT | 89 | SNN | 90 | AAWDDSLNVYV |
| YS4 | 91 | RDIRND | 92 | GAS | 93 | HRLNSYPLTFGGGTKVDIK |
| YS8 | 94 | SSDVGGYNY | 95 | DVS | 96 | SSYTSSSTPWV |
| YS7 | 97 | SLRSYY | 98 | GKN | 99 | NSRDSSGNQ |
| YS9 | 100 | SLRTYY | 101 | GKN | 102 | NSRDSSGNHVV |
| YS10 | 103 | GSDVGSYNY | 104 | EVS | 105 | SSYTTSSTLV |
| YS11 | 106 | SLRSYY | 107 | GEN | 108 | NSWDSSGNHVV |
| 3G7HY | 109 | QSISSY | 110 | AAS | 111 | QQSYSTPRT |
| 3G7NY | 112 | QSLLHSNGYDY | 113 | LGS | 114 | MQGLQTPS |
| 3G7 | 115 | SLRSYY | 116 | GKN | 117 | NSRDSSSTHRGV |
| SB2 | 118 | RSISTY | 119 | DAS | 120 | QQSYNPPWT |
| 2C8 | 121 | SSDVGGYNY | 122 | DVS | 123 | SSYTSSSDPWV |
| UA8kappa | 124 | QPISTY | 125 | GAS | 126 | QQSYSSLLTFGDGTKVEIK |

In some embodiments, said second anti-cancer agent comprises one or more CDR sequences in Table 1 or Table 2. In some embodiments, said second anti-cancer agent comprises one or more CDR sequences selected from SEQ ID NOs: 1-126. In some embodiments, said second anti-cancer agent comprises one or more CDR sequences from YS5FL, YS12, or SB1HGNY. In some embodiments, the one or more CDR sequences comprise any of SEQ ID NOs: 1-3, 10-15, 64-66 or 73-78. In some embodiments, said second anti-cancer agent comprises a heavy chain (HC) variable region that comprises three CDRs: HC CDR1, HC CDR2 and HC CDR3 and a light chain (LC) variable region that comprises three CDRs: LC CDR1, LC CDR2, and LC CDR3, wherein said HC CDR1, HC CDR2, HC CDR3 comprise an amino acid sequence of SEQ ID NO: 1, SEQ ID NO: 2, and SEQ ID NO: 3, respectively, and said LC CDR1, LC CDR2, and LC CDR3 comprise an amino acid sequence of SEQ ID NO: 64, SEQ ID NO: 65, and SEQ ID NO: 66, respectively.

In some embodiments, a CDR described herein comprises one, two, or three amino acid modifications. In some embodiments, said modification is a substitution, addition, or deletion. In some embodiments, a CDR described herein comprises one, two, or three conservative amino acid substitutions. In some embodiments, the one, two, or three amino acid modifications does not substantially modify binding to human CD46. In some embodiments, the one, two, or three amino acid modifications modifies binding to human CD46. In some embodiments, a VH-CDR3 and/or VL-CDR3 comprises an amino acid substitution that modifies binding to human CD46, immunogenicity, or some other feature. In some embodiments, the amino acid substitution is an alanine (A).

### Variable Heavy and Variable Light Regions

In some embodiments, the CD46 binding recombinant antibody comprises a VH that comprises an amino acid sequence disclosed in Table 3 or a sequence substantially identical thereto (e.g., a sequence that has at least 90%, 95%, 96%, 97%, 98%, or 99% sequence identity).

In some embodiments, the CD46 binding recombinant antibody comprises a VL that comprises an amino acid sequence disclosed in Table 4 or a sequence substantially identical thereto (e.g., a sequence that has at least 90%, 95%, 96%, 97%, 98%, or 99% sequence identity).

In some embodiments, the CD46 binding recombinant antibody comprises a VH that comprises an amino acid sequence disclosed in Table 3 or a sequence substantially identical thereto (e.g., a sequence that has at least 90%, 95%, 96%, 97%, 98%, or 99% sequence identity); and a VL that comprises an amino acid sequence disclosed in Table 4 or a sequence substantially identical thereto (e.g., a sequence that has at least 90%, 95%, 96%, 97%, 98%, or 99% sequence identity).

In some embodiments, the CD46 binding recombinant antibody comprises a VH that comprises an amino acid sequence of SEQ ID NO: 127, or a sequence substantially identical thereto (e.g., a sequence that has at least 90%, 95%, 96%, 97%, 98%, or 99% sequence identity).

In some embodiments, the CD46 binding recombinant antibody comprises a VL that comprises an amino acid sequence of SEQ ID NO: 148, or a sequence substantially identical thereto (e.g., a sequence that has at least 90%, 95%, 96%, 97%, 98%, or 99% sequence identity).

In some embodiments, the CD46 binding recombinant antibody comprises a VH that comprises an amino acid sequence of SEQ ID NO: 127, or a sequence substantially identical thereto (e.g., a sequence that has at least 90%, 95%, 96%, 97%, 98%, or 99% sequence identity); and a VL that comprises an amino acid sequence of SEQ ID NO: 148, or a sequence substantially identical thereto (e.g., a sequence that has at least 90%, 95%, 96%, 97%, 98%, or 99% sequence identity).

In some embodiments, the CD46 binding recombinant antibody comprises a VH that comprises an amino acid sequence of SEQ ID NO: 130, or a sequence substantially identical thereto (e.g., a sequence that has at least 90%, 95%, 96%, 97%, 98%, or 99% sequence identity).

In some embodiments, the CD46 binding recombinant antibody comprises a VL that comprises an amino acid sequence of SEQ ID NO: 151, or a sequence substantially identical thereto (e.g., a sequence that has at least 90%, 95%, 96%, 97%, 98%, or 99% sequence identity).

In some embodiments, the CD46 binding recombinant antibody comprises a VH that comprises an amino acid sequence of SEQ ID NO: 130, or a sequence substantially identical thereto (e.g., a sequence that has at least 90%, 95%, 96%, 97%, 98%, or 99% sequence identity); and a VL that comprises an amino acid sequence of SEQ ID NO: 151, or a sequence substantially identical thereto (e.g., a sequence that has at least 90%, 95%, 96%, 97%, 98%, or 99% sequence identity).

In some embodiments, the CD46 binding recombinant antibody comprises a VH that comprises an amino acid sequence of SEQ ID NO: 131, or a sequence substantially identical thereto (e.g., a sequence that has at least 90%, 95%, 96%, 97%, 98%, or 99% sequence identity).

In some embodiments, the CD46 binding recombinant antibody comprises a VL that comprises an amino acid sequence of SEQ ID NO: 152, or a sequence substantially identical thereto (e.g., a sequence that has at least 90%, 95%, 96%, 97%, 98%, or 99% sequence identity).

In some embodiments, the CD46 binding recombinant antibody comprises a VH that comprises an amino acid sequence of SEQ ID NO: 131, or a sequence substantially identical thereto (e.g., a sequence that has at least 90%, 95%, 96%, 97%, 98%, or 99% sequence identity); and a VL that comprises an amino acid sequence of SEQ ID NO: 152, or a sequence substantially identical thereto (e.g., a sequence that has at least 90%, 95%, 96%, 97%, 98%, or 99% sequence identity).

Using the amino acid sequences provided for the antibodies in Tables 3 and 4, numerous antibody forms can be prepared. Such forms include, but are not limited to a substantially intact immunoglobulins (e.g., an IgA, IgE, IgG, and the like), antibody fragments (e.g., Fv, Fab, (Fab′)2, (Fab′)3, IgGACH2, minibodies, and the like), single chain antibodies (e.g., scFv), diabodies, unibodies, affibodies, and the like.

TABLE 3
Amino acid sequence of anti-CD46 variable heavy chain binding domains

| Antibody | SEQ ID NO | Amino Acid Sequence |
|---|---|---|
| YS5FL | 127 | QVQLVQSGGGVVQPGRSLRLACAASGLTVNNYAMHWVRQAPGKGL |
| | | EWVAVISYDGNNKYYADSVKGRFTISRDNSKNTLYLQMNSLRAEDT |
| | | AVYYCAKGGGYFDLWGRGTLVTVSS |
| YS5F | 128 | QVQLVQSGGGVVQPGRSLRLACAASGFTVNNYAMHWVRQAPGKGL |
| | | EWVAVISYDGNNKYYADSVKGRFTISRDNSKNTLYLQMNSLRAEDT |
| | | AVYYCAKGGGYFDLWGRGTLVTVSS |
| YS5vID | 129 | QVQLVQSGGGVVQPGRSLRLACAASGFTVNNYAMHWVRQAPGKGL |
| | | EWVAVISYDGNNKYYADSVKGRFTISRDNSKNTLYLQMNSLRAEDT |
| | | AVYYCAKGGGYFDLWGRGTLVTVSS |
| SB1HGNY | 130 | QVQLQQSGGGVVQPGRSLRLSCAASGFTFSSYAMHWVRQAPGKGLE |
| | | WVAFIRSDGSKKYYADSVKGRFTISRDNSKNTLYLQMNSLRAEDTAV |
| | | YYCARHGNYFDSWGQGTLVTVSS |
| YS12 | 131 | QVQLVESGGGVVQPGRSLRLSCAASGFTFSTYGMHWVRQAPGKGLE |
| | | WLSFISYDGDEKYYADSVKGRFTISRDNSKNTLYLQMNSLRAEDTAV |
| | | YWCAKASGYGMGILDYWGQGTLVTVSS |
| 3G7RY | 132 | EVQLVESGGGLVQPGGSLRLSCAASGFTFSDYYMSWIRQAPGKGLE |
| aka 3G8 | | WVSYISSSGSTIYYADSVKGRFTISRDNSKNTLYLQMNSLRAEDTAVY |
| | | YCARDYGRIAAAGRRYWGQGTLVTVSS |
| YS6 | 133 | QVQLQESGGGVVRPGGSLRLSCAASGFTFSDYYMSWIRQAPGKGLE |
| | | WVSYISSSGSTIYYADSVKGRFTISRDNSKNTLYLQMNSLRAEDTAVY |
| | | YCARDYGRIAAAGRHYWGQGTLVTVSS |
| YS1 | 134 | EVQLVESGGGLVQPGGSLRLSCAASGFTFSDYYMSWIRQAPGKGLE |
| | | WVSYISSSGSTIYYADSVKGRFTISRDNSKNTLYLQMNSLRAEDTAVY |
| | | YCARDYGRIAAAGRHYWGQGTLVTVSS |
| YS3 | 135 | QVQLQESGGGLVQPGGSLRLSCAASGFTFSSYWMSWVRQAPGKGLE |
| | | WVADIKQDGSEKYYVDSVKGRFTISGDNAKNSLYLQMNSLRAEDTA |
| | | VYYCAKDVGSTAINYVRAYTWFDPWGQGTLVTVSS |
| YS4 | 136 | QVQLQESGGGLVQPGGSLRLSCAASGFTFSNYAMSWVRQAPGKGLE |
| | | WVSTISGSGSSTFYVDSVKGRFTISRDNSKNTLYLQMNSLRAEDTAVY |
| | | YCAQGLYSSGWANWFDPRGQGTLVTVSS |
| YS8 | 137 | QVQLQESGGGVVQPGRSLRLSCAASGFTFSSYGMHWVRQAPGKGLE |
| | | WVAVISYDGSNKYYADSVKGRFTISRDNSKNTLYLQMNSLRAEDTA |
| | | VYYCAKVMGLAAAGLDAFDIWGQGTTVTVSS |
| YS7 | 138 | QVQLVQSGGGVVQPGRSLRLSCAASGFTFSSYAMHWVRQAPGKGLE |
| | | WVAVISYDGSNKYYADSVKGRFTISRDTSTNTLYLQMNSLRADDTA |
| | | VYYCGRESSGSPGVWGQGTTVTVSS |
| YS9 | 139 | QVQLVESGGGLIQPGGSLRLSCAASGFTVSSNYMSWVRQAPGKGLE |
| | | WVSVIYTDGSTYYADSVKGRFTISRDNSKNTLYLQMNSLRAEDTAIY |
| | | YCARDRGTSGYDWAWFDLWGQGTLVTVSS |
| YS10 | 140 | QVQLQESGGGLVQPGGSLRLSCAASGFTFSSYAMSWVRQAPGKGLE |
| | | WVSAISGSGGSTYYADSVKGRFTISRDNSKNTLYMQMNSLRAEDTAV |
| | | YYCAKDRYYYGSGKDAFDIWGRGTMVTVSS |
| YS11 | 141 | QVQLVESGGGLVQPGGSLGLSCAASGFTFSNYWMSWVRQAPGKGLE |
| | | WVANVRQDGGQKYYVDSVKGRFTISRDNAKNSLYLQMNSLRTEDT |
| | | AVYFCVSQRNSGEHDYWGQGTLVTVSS |
| 3G7HY | 142 | EVQLVESGGGLVQPGGSLRLSCAASGFTFSDYYMSWIRQAPGKGLE |
| | | WVSYISSSGSTIYYADSVKGRFTISRDNSKNTLYLQMNSLRAEDTAVY |
| | | YCARDYGRIAAAGRHYWGQGTLVTVSS |
| 3G7NY | 143 | EVQLVESGGGLVQPGGSLRLSCAASGFTFSDYYMSWIRQAPGKGLE |
| | | WVSYISSSGSTIYYADSVKGRFTISRDNSKNTLYLQMNSLRAEDTAVY |
| | | YCARDYGRIAAAGRNYWGQGTLVTVSS |
| 3G7 | 144 | QVQLQESGGGVVRPGGSLRLSCAASGFTFSDYYMSWIRQAPGKGLE |
| | | WVSYISSSGSTIYYADSVKGRFTISRDNSKNTLYLQMNSLRAEDTAVY |
| | | YCARDYGRIAAAGRHYWGQGTLVTVSS |
| SB2 | 145 | EVQLVESGGGLVKPGGSLRLSCAASGFTFSDYYMSWIRQAPGKGLE |
| | | WVSYISSSGSSIYYADSVKGRFTISRDNAKNSLYLQMNSLKAEDTAVY |
| | | YCARDITDVVGVSFDYWGQGTLVTVSS |
| 2C8 | 146 | EVQLVESGGGVVQPGRSLRLSCAASGFTFSSYGMHWVRQAPGKGLE |
| | | WVAVISYDGSNKYYADSVKGRFTISRDNSKNTLYLQMNSLRAEDTA |
| | | EYYCAKVMGLAAAGLDAFDIWGQGTLVTVSS |
| UA8kappa | 147 | EVQLVESGGGVVQPGRSLRLSCAASGFTFSSFGMHWVRRAPGKGLE |
| | | WVAVISYDGSNQYYADSVKGRFTISRDNSKNTLYLQMNSLRAEDTA |
| | | VYYCGSRPGGGYASGSTVAYWGQGTLVTVSS |

TABLE 4
Amino acid sequence of anti-CD46 variable light chain binding domains

| | SEQ ID | |
|---|---|---|
| Antibody | NO | Amino Acid Sequence |
| YS5FL | 148 | QSVLTQPPSVSGAPGQRVTISCTGSSSNIGAGYDVHWYQQLPGTAPKL |
| | | LIYGNNNRPSGVPDRFSGSKSGTSASLAITGLQAEDEADYYCSSYTSGT |
| | | WLFGGGTKLTVL |
| YS5F | 149 | QSVLTQPPSVSGAPGQRVTISCTGSSSNIGAGYDVHWYQQLPGTAPKL |
| | | LIYGNNNRPSGVPDRFSGSKSGTSASLAITGLQAEDEADYYCSSYTSGT |
| | | WLFGGGTKLTVL |
| YS5vID | 150 | QSVLTQPPSVSGAPGQRVTISCTGSSSNIGAGYDVHWYQQLPGTAPKL |
| | | LIYGDNNRPSGVPDRFSGSKSGTSASLAITGLQAEDEADYYCSSYTSGT |
| | | WLFGGGTKLTVL |
| SB1HGNY | 151 | DIQMTQSPSFLSASVGDRVTITCRASQGISSYLAWYQQKPGKAPKLLIY |
| | | AASTLQSGVPSSFSGSGSGTEFTLTISSLQPEDFATYYCQQLASYPLTFG |
| | | GGTKVDIK |
| YS12 | 152 | SSELTQDPAVSVALGQTVRITCQGDSLRSYYVSWFQQKPGQAPVFVM |
| | | YGQNNRPSGISERFSGSSSGNTASLIITGAQAEDEADYYCHSRDSSGTH |
| | | LRVFGGGTKLTVL |
| 3G7RY | 153 | QSALTQPPSASATPGQRVTISCSGRTSNIGSNHVYWYQQLPGTAPKLLI |
| aka 3G8 | | YRNNQRPSGVPDRFSGSKSGTSASLAISGLRSEDEADYYCATWDDSLS |
| | | GEVFGGGTKLTVL |
| YS6 | 154 | SSELTQDPAVSVALGQTVRITCQGDSLRSYYASWYQQKPGQAPVLVIY |
| | | GKNNRPSGIPDRFSGSSSGNTASLTITGAQAEDEADYYCNSRDSSGTHL |
| | | EVFGGGTKVTVL |
| YS1 | 155 | SSELTQDPAVSVALGQTVRITCQGDTLSTYYANWYQQKPGQAPVLVIY |
| | | GKNNRPSGIPDRFSGSSSGNTASLTITGAQAEDEADYYCHSRDISGNYL |
| | | FASGTKLTVL |
| YS3 | 156 | QSVLTQPPSASGTPGQRVTISCSGSSSNIGSNTVNWSRQLPGTAPKLLIY |
| | | SNNQRPSGVPDRFSGSKSGTSASLAISGLQSEDEADYYCAAWDDSLNV |
| | | YVFGTGTKVTVL |
| YS4 | 157 | KIVLTQSPSSLSASVGDTVTIACRASRDIRNDLAWYQQKPGKAPKLLIY |
| | | GASSLQSGVPSRFSGSGSGTEFILTISSLQPEDFATYYCHRLNSYPLTFG |
| | | GGTKVDIK |
| YS8 | 158 | NFMLTQPASLSGSPGQSITISCTGTSSDVGGYNYVSWYQQHPGYAPKL |
| | | MIYDVSNRPSGVSNRFSGSKSGNTASLTISGLQAEDEADYYCSSYTSSS |
| | | TPWVFGGGTKLTVL |
| YS7 | 159 | SYVLTQDPAVSVALGQTVRITCQGDSLRSYYASWYQQKPGQAPVLVI |
| | | YGKNNRPSGIPDRFSGSSSGNTASLTITGAQAEDEADYYCNSRDSSGNQ |
| | | FGGGTKLTVL |
| YS9 | 160 | SSELTQDPAVSVALGQTVRITCQGDSLRTYYASWYQQRPGQAPILVLY |
| | | GKNNRPSGIPDRFSGSSSGNTASLTITGAQAEDEADYYCNSRDSSGNHV |
| | | VFGGGTKLTVL |
| YS10 | 161 | QSVLTQPASVSGSPGQSITISCTGTGSDVGSYNYVSWYQQNPGKAPKL |
| | | MIYEVSNRPSGVSNRFSGSKSGNTASLTISGLQAEDEADYYCSSYTTSS |
| | | TLVFGGGTKVTVL |
| YS11 | 162 | SELTQDPAVSVALGQTVRITCQGDSLRSYYASWYQQKPGQAPVLVIYG |
| | | ENSRPSGIPDRFSGSSSGNTASLTITGAQAEDEADYYCNSWDSSGNHVV |
| | | FGGGTKLTVL |
| 3G7HY | 163 | AIRMTQSPSSLSASVGDRVTITCRASQSISSYLNWYQQKPGKAPKLLIY |
| | | AASSLQSGVPSRFSGSGSGTDFTLTISSLQPEDFATYYCQQSYSTPRTFG |
| | | QGTKLEIK |
| 3G7NY | 164 | DIVMTQSPLSLPVTPGEPASISCRSSQSLLHSNGYDYLDWYLQKPGQSP |
| | | QLLIYLGSNRASGVPDRFSGSGSGTDFTLKISRVETEDVGIYYCMQGLQ |
| | | TPSFGQGTKLEIK |
| 3G7 | 165 | SSELTQDPAVSVALGQTVRITCQGDSLRSYYASWYQQKPGQAPVPVIY |
| | | GKNNRPSGIPDRFSGSSSGNTASLTITGAQAEDEADYYCNSRDSSSTHR |
| | | GVFGGGTKLTVL |
| SB2 | 166 | DIQLTQSPSSLSASVGDRVTITCRASRSISTYLSWYQQKPGKAPKLLIYD |
| | | ASRLQNGVPSRFSGSGSDTDFTLTISSLQPEDFATYFCQQSYNPPWTFG |
| | | QGTKLEIK |
| 2C8 | 167 | QSALTQPASVSGSPGQSITISCTGTSSDVGGYNYVSWYQQHPGKAPKL |
| | | MIYDVSNRPSGVSNRFSGSKSGNTASLTISGLQAEDEAYYYCSSYTSSS |
| | | DPWVFGGGTQLTVL |
| UA8kappa | 168 | NIQMTQSPSSLSASVGDRVTITCRAGQPISTYVNWYQHKPGKAPKLLIY |
| | | GASNLQSGVPSRFSGGGSATDFTLTISSLQPEDFATYYCQQSYSSLLTFG |
| | | DGTKVEIK |

### Heavy Chain and Light Chains

In some embodiments, the CD46 binding recombinant antibody comprises a heavy chain that comprises an amino acid sequence disclosed in Table 5 or a sequence substantially identical thereto (e.g., a sequence that has at least 90%, 95%, 96%, 97%, 98%, or 99% sequence identity).

In some embodiments, the CD46 binding recombinant antibody comprises a light chain that comprises an amino acid sequence disclosed in Table 6 or a sequence substantially identical thereto (e.g., a sequence that has at least 90%, 95%, 96%, 97%, 98%, or 99% sequence identity).

In some embodiments, the CD46 binding recombinant antibody comprises a heavy chain that comprises an amino acid sequence disclosed in Table 5 or a sequence substantially identical thereto (e.g., a sequence that has at least 90%, 95%, 96%, 97%, 98%, or 99% sequence identity); and a light chain that comprises an amino acid sequence disclosed in Table 6 or a sequence substantially identical thereto (e.g., a sequence that has at least 90%, 95%, 96%, 97%, 98%, or 99% sequence identity).

In some embodiments, CD46 binding recombinant antibody comprises a heavy chain that comprises an amino acid sequence of SEQ ID NO: 169, or a sequence substantially identical thereto (e.g., a sequence that has at least 90%, 95%, 96%, 97%, 98%, or 99% sequence identity).

In some embodiments, the CD46 binding recombinant antibody comprises a light chain that comprises an amino acid sequence of SEQ ID NO: 170, or a sequence substantially identical thereto (e.g., a sequence that has at least 90%, 95%, 96%, 97%, 98%, or 99% sequence identity).

In some embodiments, the CD46 binding recombinant antibody comprises a heavy chain that comprises an amino acid sequence of SEQ ID NO: 169, or a sequence substantially identical thereto (e.g., a sequence that has at least 90%, 95%, 96%, 97%, 98%, or 99% sequence identity); and a light chain that comprises an amino acid sequence of SEQ ID NO: 170, or a sequence substantially identical thereto (e.g., a sequence that has at least 90%, 95%, 96%, 97%, 98%, or 99% sequence identity).

TABLE 5
Amino acid sequence of anti-CD46 heavy chain

| Name | SEQ ID NO | Amino Acid Sequence |
|---|---|---|
| YS5FL | 169 | QVQLVQSGGGVVQPGRSLRLACAASGLTVNNYAMHWVRQAPGKGLEWVAVISY |
| | | DGNNKYYADSVKGRFTISRDNSKNTLYLQMNSLRAEDTAVYYCAKGGGYFDLW |
| | | GRGTLVTVSSASTKGPSVFPLAPSSKSTSGGTAALGCLVKDYFPEPVTVSWNSGAL |
| | | TSGVHTFPAVLQSSGLYSLSSVVTVPSSSLGTQTYICNVNHKPSNTKVDKKVEPKSC |
| | | DKTHTCPPCPAPELLGGPSVFLFPPKPKDTLMISRTPEVTCVVVDVSHEDPEVKFN |
| | | WYVDGVEVHNAKTKPREEQYNSTYRVVSVLTVLHQDWLNGKEYKCKVSNKALP |
| | | APIEKTISKAKGQPREPQVYTLPPSREEMTKNQVSLTCLVKGFYPSDIAVEWESNGQ |
| | | PENNYKTTPPVLDSDGSFFLYSKLTVDKSRWQQGNVFSCSVMHEALHNHYTQKSL |
| | | SLSPGK |

TABLE 6
Amino acid sequence of anti-CD46 light chain

| Name | SEQ ID NO | Amino Acid Sequence |
|---|---|---|
| YS5FL | 170 | QSVLTQPPSVSGAPGQRVTISCTGSSSNIGAGYDVHWYQQLPGTAPKLLIYGNNNR |
| | | PSGVPDRFSGSKSGTSASLAITGLQAEDEADYYCSSYTSGTWLFGGGTKLTVLGQP |
| | | KAAPSVTLFPPSSEELQANKATLVCLISDFYPGAVTVAWKADSSPVKAGVETTTPS |
| | | KQSNNKYAASSYLSLTPEQWKSHRSYSCQVTHEGSTVEKTVAPTECS |

In some embodiments, the anti-CD46 antibody disclosed herein comprises an immunoglobulin constant region (e.g., an Fc region). Exemplary Fc regions can be chosen from the heavy chain constant regions of IgG1, IgG2, IgG3 or IgG4; more particularly, the heavy chain constant region of human IgG1 or IgG4. In some embodiments, the immunoglobulin constant region (e.g., the Fc region) is altered, e.g., mutated, to increase or decrease one or more of: Fc receptor binding, antibody glycosylation, the number of cysteine residues, effector cell function, or complement function. In some embodiments, the second anti-cancer agent comprises a constant region of an IgG heavy chain. In some embodiments, said second anti-cancer agent comprises a constant region of an IgG1 heavy chain.

### Additional Antibody Sequences

In some embodiments, the antibodies contemplated herein includes or excludes antibodies comprising the three VH CDRs and/or the three VL CDRs of antibodies 3051.1, G12FC3, M6c42b, 4F3YW, M40pr146, UA20, UA8, 5851141, 5851141.1, 5851156, 3076, 3051, M49R, RCI-14, 1179 4, 1179 3, T511-4B.1, T51I-4B.2, RCI-11, RCI-20, CI-11A, CI-14A, S95-2 that are described in PCT/US2008/076704 (WO 2009/039192) and/or the mPA7 antibody. The amino acid sequences of the VH and VL chains of these antibodies and the CDRs comprising these domains are shown in in PCT/US2008/076704 and the amino acid sequences of these domains are reproduced below in Table 7. The sequence shown in Table 7 may be included in scFv antibodies. (e.g., the VL and VH regions may be joined by a GGGGSGGGGSGGGGS linker (SEQ ID NO: 194)); however it will be recognized that other antibody forms comprising the CDRs (or the VH and/or VL domains) may also be included or excluded.

In some embodiments, the anti-CD46 antibody or antibody fragment comprises an amino acid sequence disclosed in Table 7 or a sequence substantially identical thereto (e.g., a sequence that has at least 90%, 95%, 96%, 97%, 98%, or 99% sequence identity).

TABLE 7
Additional antibodies

| | SEQ ID | |
|---|---|---|
| Name | NO | Amino Acid Sequence |
| 3051.1 | 171 | QVQLQESGGGLVKPGGPLRLSCAASGFTFSSYGMYWVRQAPGKGLE |
| | | WVSTLSRSGSGTYYADSVKGRFTISRDNSKNTLYLQMNSLRAEDTAV |
| | | YYCASIAVAGNYFDYWGQGTLVTVSSGGGGSGGGGSGGGGSSYVL |
| | | TQDPAVSVALGQTVRITCQGDSLRSYYASWYQERPGQAPLLVIYGKN |
| | | NRPSGIPDRFSGSNSGSTATLTISRVEAGDEGDYYCQVWDSINEQVVFG |
| | | GGTKVTVL |
| G12FC3 | 172 | QVQLVQSGGGVVQPGRSLRLSCAATGIPFSGSGMHWVRQAPGKGLE |
| | | WVTMIWYDGSNKFYADSVKGRFTISRDNSKNTLYLQMDSLRAEDTAV |
| | | YFCARDKGVRSMDVWGLGTTVTVSSGGGGSGGGGSGGGGSNFMLT |
| | | QPPSVSVAPGQTAKITCDGYSIRTKSVHWYQQKPGQAPVVVVHDDSD |
| | | RPSGIPERFSGSNSGTTATLTISRVEAGDEADYYCQAWDSISEEVVFGG |
| | | GTKLTVL |
| M6c42b | 173 | QVQLQESGGGLVQPGGSLRLSCSASGFTFGTYAMRWVRQTSGKGLEW |
| | | VSGIGVSGDAYYTDSVRGRFTISRDNSKNTLYLQMNTLRAEDTATYYC |
| | | TRKSSTTSNDYWGRGTLVTVSSGGGGSGGGGSGGGGSSYVLTQDPA |
| | | VSVALGQTVRITCQGDNIGSKSVHWYQQKPGQAPVLVVYDDSDRPSG |
| | | IPERFSGSNSGTTATLTISSVEAGDEADYYCQAWDSISEHVIFGGGTKV |
| | | TVL |
| 4F3YW | 174 | QVQLQESGGGLVQPGGSLRLSCAASGFTFSSYAMHWVRQAPGKGLE |
| | | WVAVISYDGSNKYYADSVKGRFTISRDNSKNTLYLQMNSLRAEDTAV |
| | | YYCARFSSGWYYFDYWGQGTLVTVSSGGGGSGGGGSGGGGSDIQM |
| | | TQSPSFLSASVGDRITITCRASHDISSYFAWYQQKPGKAPKPLIYAASTL |
| | | QSGVPSRFSGSGSGTEFTLTISSLQPEDFATYYCQQLGSYPLTFGGGTKL |
| | | EIK |
| M4Opr146 | 175 | QVQLLQSGGGLVQPGGSLRLSCAASGFTFSSYAMSWVRQAPGKGLEW |
| | | VSAISGSGGSTYYTDSVKGRFTISRDNSKNTLYLQMNSLRAEDTAVYY |
| | | CAKSHDYGDYAGFDYWGQGTLVTVSSGGGGSGGGGSGGGGSHVIL |
| | | TQDPAVSVALGQTVRITCQGDSLKSYYASWYQQKPGQAPVLVIYGKN |
| | | NRPSGIPDRFSGSSSGTTASLTITGAQAEDEADYYCHSRDSSGTHLRVF |
| | | GGGTKLTVL |
| UA20 | 176 | QVQLQESGGGLVKPGGSLRLSCAASGFTFSNAWMNWVRQAPGKGLE |
| | | WVGRIKSKTDEGTTDYAAPVKGRFSISRDDSKNTLYLQMNSLKTEDTG |
| | | VYYCTATKGLGGSKLGQGTLVTVSSGGGGSGGGGSGGGGSQSVLTQ |
| | | PPSASGTPGQRVTISCSGSSSNIGNNTVNWSRQLPGTAPKLLIYSNDQRP |
| | | SGVPDRFSGSKSGTSASLAITGLQPEDEADYYCGTWDSSLSAYVFGTG |
| | | TKLTVL |
| UA8 | 177 | QVQLVESGGGVVQPGRSLRLSCAASGFTFSSFGMHWVRRAPGKGLEW |
| | | VAVISYDGSNQYYADSVKGRFTISRDNSKNTLYLQMNSLRAEDTAVY |
| | | YCGSRPGGGYASGSTVAYWGQGTPVTVSSGGGGSGGGGSGGGGSSS |
| | | ELTQDPAVSVALGQTVRITCQGDSLRSYYASWYQQKPGQAPLLVIYG |
| | | QNIRPSGIPDRFSGSSSGNSASLTITGAQAEDEADYYCHSRDSSGKYVF |
| | | GVGTKVTVL |
| 5851141 | 178 | QVQLVESGGGLVQPGGSLRLSCAASGFTFSSYAMGWVRQAPGKGLE |
| | | WVSAISGSGGSTYYADSVKGRFTISRDNSKDTLYLQMNSLRAEDTAVY |
| | | YCASRSLLDYWGQGTLVTVSSGGGGSGGGGSGGGGSNFMLTQDPA |
| | | VSVALGQTVRITCQGDSLRSYYASWYQQKPGQAPLLVIYGKNNRPSGI |
| | | PDRFSGSSSGNTASLTITGAQAEDEADYYCNSRDSSGNPVFGGGTKVT |
| | | VL |
| 5851141.1 | 179 | QVQLVESGGGLVQPGGSLRLSCAASGFTFSSYAMSWVRQAPGKGLEW |
| | | VSAISGSGGSTYYADSVKGRFTISRDNSKDTLYLQMNSLRAEDTAVYY |
| | | CASRSLLDYWGQGTLVTVSSGGGGSGGGGSGGGGSNFMLTQDPAVS |
| | | VALGQTVRITCQGDSLRSYYASWYQQKPGQAPLLVIYGKNNRPSGIPD |
| | | RFSGSSSGNTASLTITGAQAEDEADYYCNSRDSSGNPVFGGGTKVTVL |
| 5851156 | 180 | QVQLQESGGGLVQLGGSLRLSCAASGFTFSSYAMSWVRQAPGKGLEW |
| | | VSAISGSGGSTYYADSVKGRFTISRDNSKNTLYLQMSSLRAEDTAFYY |
| | | CANSAYTGGWYDYWGHGTLVTVSSGGGGSGGGGSGGGGSSSELTQ |
| | | DPAVSVALGQTVKITCQGDSLRTYYASWYQQRPGQAPVLVIYGENSR |
| | | PSGIPDRFSGSSSGNTASLTITGAQAEDEADYYCNSRDSSGNHLRVFGG |
| | | GTKLTVL |
| 3076 | 181 | QVNLRESGGGLVQPGGFLRLSCAAFGFTFSGYWMSWVHPAPGKGLE |
| | | WVANIKQDGSEKFYVDSVKGRFTISRDNAKNSLFLQMNSLRAEDTAV |
| | | YFCARGLLSDYWGQGTLVPVSSGGGGSGGGGSGGGGSNFMLTQPPS |
| | | VSVAPGKTASLTCGGYNIGTKSVHWYQQKPGQAPVVVVHDDSDRPSG |
| | | IPERFSGSNSGTTATLTIIRVEAGDEADYYCQAWDSISEEVVFGGGTKL |
| | | TVL |
| 3051 | 182 | QVQLQESGGGLVKPGGPLRLSCAASGFTFSSYGMYWVRQAPGKGLE |
| | | WVSTLSRSGSGTYYAESVKGRFTISRDNSKNTLYFQMNSLRAEDTAVY |
| | | YCASIAVAGNYFEYWGQGTLVTVSSGGGGSGGGGSGGGGSSYVLTQ |
| | | DPAVSVALGQTVRITCQGDSLRSYYASWYQERPGQAPLLVIYGKNNRP |
| | | SGIPDRFSGSNSGSTATLTISRVEAGDEGDYYCQVWDSINEQVVFGGGT |
| | | KVTVL |
| M49R | 183 | QVQLQESGGGLVKPGESLRLSCAASGFTFSDHYMDWVRQAPGKGLE |
| | | WVAYIRYDGSTKYYADSVKGRFTISRDNSKNTLYLQMNSLRPEDTAF |
| | | YYCARLIAEAEGWFDPWGQGTLVTVSSGGGGSGGGGSGGGGSNFM |
| | | LTQPPSVSVAPGKTARITCGGNNIGSKSVYWYQQKPGQAPVLVVYDD |
| | | SDRPSGIPERFSGSNSGNTATLTISRVEAGDEADYYCQVWDSSSDHVVF |
| | | GGGTKVTVL |
| RCI-14 | 184 | QVQLLQSAGGLVQPGGSLRLSCAASGFTFSTYAMNWVRQAPGKGLE |
| | | WVSGISGSGGSTNYADSVKGRFTISRDSSKNTLFLQMNSLRAEDTAVY |
| | | YCAKDYGSGWYDYWGQGTLVTVSSGGGGSGGGGSGGGGSSSELTQ |
| | | DPAVSVALGQTVRITCQGDSLRSYYASWYQERPGQAPLLVIYGRNERP |
| | | SGIPDRFSASSSGNTASLTITGAQAEDEADYYCQVWDSFNEQVVFGGG |
| | | TKLTVL |
| 1179_4 | 185 | QVQLVESGGGLVQPGGSLRLSCAASGFTFSSYAMSWVHQAPGKGLEW |
| | | VSAISGSGGSTYYADSVKGRFTISRDNSKNTLYLQMNSLRAEDTAVYY |
| | | CAKTYYGFWSGYYDYLGQGTLVTVSSGGGGSGGGGSGGGGSSSELT |
| | | QDPAVSVGLGQTVTITCQGDSLRSYYANWYQQKPGQAPILVIYGENN |
| | | RPSGIPDRFSGSSSGNTASLTITGAQAEDEADYYCHSRDSSGTHLRVFG |
| | | GGTKLTVL |
| 1179_3 | 186 | QVQLLESGGGVVQPGTSLRLSCAASGFTFSNYAINWVRQAAGKGLEW |
| | | VSGISGSGVSTSYADSVKGRFTVSRDNSKNTLYLQMNSLRVEDTALYY |
| | | CAKNGGGPEYLQHWGQGTLVTVSSGGGGSGGGGSGGGGSQSVLTQ |
| | | PPSASGTPGQRVTISCSGSSSNIGNNTVNWSRQLPGTAPKLLIYSNDQRP |
| | | SGVPDRFSGSKSGTSASLAITGLQPEDEADYYCGTWDSSLSAYVFGTG |
| | | TKLTVL |
| T511-4B.1 | 187 | QVQLQESGGTLVQPGGSLRLSCAASGFTFSSYAMSWVRQAPGRGLEW |
| | | VSTISGSGGSTYYADSVKGRFTISRDNSKNTLYLQMNSLRAEDTAVYY |
| | | CAKGAYSGSYWGQGTLVTVSSGGGGSGGGGSGGGGSSSELTQDPAV |
| | | SVALGQTVRITCQGDSLRSYYASWYQQKPGQAPSLVIYGENSRPSGIPD |
| | | RFSGSSSGNTASLTITGAQAENEADYYCQAWDSSTAVVFGGGTKLTVL |
| T51I-4B.2 | 188 | QVQLQESGGTLVQPGGSLRLSCAASGFTFSSYAMSWVRQAPGRGLEW |
| | | VSTISGSGGSTYYADSVKGRFTISRDNSKNTLYLQMNSLRAEDTAVYY |
| | | CAKGAYSGSHWGQGTLVTVSSGGGGSGGGGSGGGGSSSELTQDPAV |
| | | SVALGQTVRITCQGDSLRSYYASWYQQKPGQAPSLVIYGENSRPSGIPD |
| | | RFSGSSSGNTASLTITGAQAENEADYYCQAWDSSTAVVFGGGTKLTVL |
| RCI-11 | 189 | QVQLVESGAEVKKPGASVKVSCKASGYTFTSYGISWVRQAPGQGLEW |
| | | MGWISAYNGNTNYAQKLQGRVTMTTDTSTSTAYMELRSLRSDDTAV |
| | | YYCARPIYDSSGYDAFDIWGQGTMVTVSSGGGGSGGGGSGGGGSDI |
| | | VMTQSPSTLSASIGDRVTITCRASEGIYHWLAWYQQKPGKAPKLLIYK |
| | | ASSLASGAPSRFSGSGSGTDFTLTISSLQPDDFATYYCQQYHTISRTFGP |
| | | GTKVDIK |
| RCI-20 | 190 | QVQLVESGGGLVKPGGSLRLSCAASGFTFSSYAMHWVRQAPGKGLE |
| | | WVAVISYDGSNKYYADSVKGRFTISRDNSKNTLYLQMNSLRAEDTAV |
| | | YFCVRPSDSGWSFEHWGQGTLVPVSSGGGGSGGGGGGGGSQSVLT |
| | | QPPSASGTPGQRVTISCSGSSSNIGNNTVNWSRQLPGTAPKLLIYSNDQ |
| | | RPSGVPDRFSGSKSGTSASLAITGLQPEDEADYYCGTWDSSLSAYVFGT |
| | | GTKLTVL |
| CI-11A | 191 | QVQLQESGGGLVQPGGSLRLSCAASGFTFSSYAMSWVRQAPGKGLEW |
| | | VAVISYDGSNKYYADSVKGRFTISRDNSKNTLYLQMNSLRAEDTAVY |
| | | YCVRGDRSYGAEYFQHWGQGTLVTVSSGGGGSGGGGSGGGGSSSE |
| | | LTQDPAVSVASGQTVRITCQGDSLRSYYASWYQQKPGQAPLLVIYGK |
| | | NIRPSGIPDRFSGSTSGNSASLTITGAQAEDEADYYCNSRDSSGNRNWV |
| | | FGGGTKLTVL |
| CI-14A | 192 | QVQLQESGGGLVKPGGSLRLSCAASGFTSSSYAMHWVRQAPGKGLEY |
| | | VSAIGGNGGTYYADSVKGRFTISRDNSKNTLYLQMNSLRAEDTAVYY |
| | | CAKEGEQWLEYRYYYGMDVWGQGTTVTVSSGGGGSGGGGSGGGG |
| | | SSSELTQDPAVSVALGQTVRITCQGDSLRSYYASWYQQKPGQAPSLVI |
| | | YGENSRPSGIPDRFSGSSSGNTASLTITGAQAENEADYYCQAWDSSTA |
| | | VVFGGGTKLTVL |
| S95-2 | 193 | QVQLVESGGGVVQPGRSLRLSCTASGFTFSSYGMHWVRQAPGKGLE |
| | | WVAVISYDGSNKYYADSVKGRFTISRDNSKNTLYLQMNSLRAEDTAV |
| | | YYCARGGRYSSNWFSYYYYGMDVWGQGTTVTVSSGGGGSGGGGS |
| | | GGGGSNFMLTQPPSVSVAPGKTARITCGGNNIGSKSVYWYQQKPGQA |
| | | PVLVVYDDSDRPSGIPERFSGSNSGNTATLTISRVEAGDEADYYCQVW |
| | | DSSSDHVVFGGGTKVTVL |

### Effector Agents

In some embodiments, disclosed herein are immunoconjugates that comprise an anti-CD46 antibodies attached to an effector agent (or prodrug thereof). In some embodiments, a second anti-cancer agent includes a cytotoxic effector coupled to an antibody that specifically binds CD46, or coupled to the CD46-binding fragment thereof. The effector agent or prodrug thereof may be used in the methods of treating cancer described herein. For example, the effector agent or prodrug thereof may be included in a second anti-cancer agent. In some embodiments, the effector agent is a drug (or prodrug thereof), small molecule, protein, peptide, antibody, ligand, receptor, cytotoxic agent, cytostatic agent, liposome, nanoparticle, radionuclide, cytokine, chemokine, a toxin, a detectable label, a viral particle, or a chelate.

In some embodiments, the effector agent is a drug (or prodrug thereof). In some embodiments, the effector agent is an anti-cancer agent (or prodrug thereof). In some embodiments, the effector agent is a chemotherapeutic agent (or prodrug thereof). In some embodiments, the effector agent is a microtubule inhibitor (or prodrug thereof), a DNA-damaging agent (or prodrug thereof), or a polymerase inhibitor (or prodrug thereof).

In some embodiments, the effector agent is a microtubule inhibitor (or prodrug thereof). In some embodiments, the microtubule inhibitor is an auristatin (or a derivative thereof), dolastatin-10 (or a derivative thereof), or maytansine (or a derivative thereof). In some embodiments, the microtubule inhibitor is monomethylauristatin F (MMAF), auristatin E (AE), monomethylauristatin E (MMAE), valine-citrulline MMAE (vcMMAE), or valine-citrulline MMAF (vcMMAF). In some embodiments, the microtubule inhibitor is monomethylauristatin E (MMAE).

In some embodiments, the effector agent comprises or consists of a compound of Formula A:

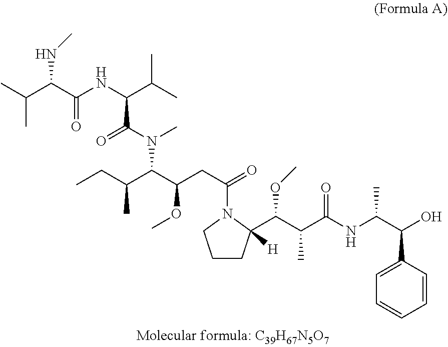 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@@H](NC)C(C)C)C(C)C]

In certain embodiments, the effector comprises a detectable label. Suitable detectable labels include, but are not limited to radio-opaque labels, nanoparticles, PET labels, MRI labels, radioactive labels, and the like. Among the radionuclides and useful in various embodiments of the present invention, gamma-emitters, positron-emitters, x-ray emitters and fluorescence-emitters are suitable for localization, diagnosis and/or staging, and/or therapy, while beta and alpha-emitters and electron and neutron-capturing agents, such as boron and uranium, also can be used for therapy.

In some embodiments, the cytotoxic effector of the second anti-cancer agent comprises a second drug or prodrug thereof, peptide, protein, liposome containing the drug or prodrug thereof, radionucleotide, viral particle, or chelate. In some embodiments, said cytotoxic effector comprises a second drug. In some embodiments, said second drug comprises an anti-cancer drug, a chemotherapeutic agent, a microtubule inhibitor, a DNA-damaging agent, or a polymerase inhibitor. In some embodiments, said second drug comprises auristatin, dolastatin-10, or maytansine, or a derivative thereof In some embodiments, said second drug comprises monomethylauristatin F (MMAF), auristatin E (AE), monomethylauristatin E (MMAE), valine-citrulline MMAE (vcMMAE), or valine-citrulline MIMAF (vcMIMAF). In some embodiments, said second drug comprises monomethylauristatin E (MMAE).

### Immunoconiugates

In one aspect, provided herein are immunoconjugates comprising an anti-CD46 antibody and an effector agent. In some embodiments, the methods described herein utilize these immunoconjugates. The immunoconjugate may be used in the methods of treating cancer described herein. For example, the immunoconjugate may be administered to a subject in need thereof as second first anti-cancer agent.

In some embodiments, the immunoconjugate comprises an anti-CD46 antibody (or antigen binding fragment thereof) described herein. In some embodiments, the immunoconjugate comprises a YS5FL antibody (or antigen binding fragment thereof).

In some embodiments, the effector agent is conjugated to the anti-CD46 antibody. In some embodiments, the effector agent is attached to the anti-CD46 antibody via a liker. In some embodiments, the linker is a peptide linker, a small molecule linker, or a linker that comprises a peptide and a small molecule. Exemplary peptide linkers include, but are not limited to, peptide linkers comprising glycine, serine, or glycine and serine.

In some embodiments, the linker is cleavable. In some embodiments, the linker is cleaved only upon internalization into a cell. In some embodiments, the cleavable linker is only cleavable upon internalization into a cancer cell. In some embodiments, the cleavable portion of a linker is a peptide (e.g., a dipeptide, e.g., ValCit). In some embodiments, the cleavable linker is cleavable by cathepsin. In some embodiments, the linker comprises maleimide. In some embodiments, the linker comprises caproic acid. In some embodiments, the linker comprises maleimide and caproic acid. In some embodiments, the linker comprises maleimide, caproic acid, and a cleavable dipeptide.

In some embodiments, the linker comprises or consists of is a maleimidocaproyl-valine-citrulline-para-amino benzyloxycarbonyl (mc-vc-PAB).

In some embodiments, the linker comprises or consists of a compound of Formula B:

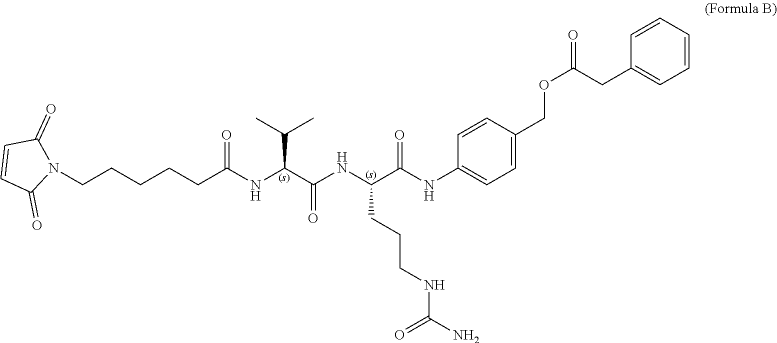 [SMILES: CC(C)[C@H](NC(=O)CCCCCN1C(=O)C=CC1=O)C(=O)N[C@@H](CCCNC(N)=O)C(=O)Nc1ccc(COC(=O)Cc2ccccc2)cc1]

In some embodiments, said cytotoxic effector is conjugated to said antibody that specifically binds CD46 via a linker. In some embodiments, said linker comprises a peptide, small molecule, or a combination thereof. In some embodiments, said linker comprises maleimidocaproyl-valine-citrulline-para-amino benzyloxycarbonyl (mc-vc-PAB).

Some embodiments include a peptide linker. Some non-limiting examples of peptide linkers include GGGGS GGGGS GGGGS (SEQ ID NO: 194), GGGGS GGGGS (SEQ ID NO: 195), GGGGS (SEQ ID NO: 196), GS GGGGS GGGGS GGS GGGGS (SEQ ID NO: 197), SGGGGS (SEQ ID NO: 198), GGGS (SEQ ID NO: 199), VPGV (SEQ ID NO: 200), VPGVG (SEQ ID NO: 201), GVPGVG (SEQ ID NO: 202), GVG VP GVG (SEQ ID NO: 203), VP GVG VP GVG (SEQ ID NO: 204), GGSSRSS (SEQ ID NO: 205), or GGSSRSSSSGGGGSGGGG (SEQ ID NO: 206). A peptide linker may be rich in glycine or serine. Some non-limiting examples of peptide linkers include GS, GGS, GGGS (SEQ ID NO: 199), or GGGGS (SEQ ID NO: 196).

An amino acid linker can comprise, for example, more than one amino acid, greater than 5 amino acids, greater than 10 amino acids, greater than 50 amino acids, or greater than 100 amino acids. A peptide linker can comprise, for example, from 1 to 100 amino acids from 3 amino acids to 75 amino acids, from 5 amino acids to 50 amino acids, or from 10 amino acids to 25 amino acids.

In some embodiments, an effector agent is attached to a light chain of the anti-CD46 antibody. In some embodiments, an effector agent is attached to a light chain constant region of the anti-CD46 antibody. In some embodiments, an effector agent is attached to a heavy chain of the anti-CD46 antibody. In some embodiments, an effector agent is attached to a heavy chain constant region of the anti-CD46 antibody.

In some embodiments, an effector moiety is attached to a cysteine residue of the anti-CD46 antibody. In some embodiments, an anti-CD46 antibody is partially reduced prior to conjugation to an effector moiety such that 1-4 interchain disulfide bonds are reduced while intrachain disulfide bonds are not reduced. Partial reduction exposes pairs of cysteine residues, rendering them accessible to conjugation to adducts such as mc-vc-PAB-MMAE. In some embodiments, the following interchain cysteine pairs of YSSFL are exposed: C219 of the first heavy chain and C214 of the first light chain; C219 of the second heavy chain and C214 of the second light chain; C225 of the first heavy chain and C225 of the second light chain; and C228 of the first heavy chain and C228 of the second light chain. In some embodiments, an effector such as mc-vc-PAB-MMAE is conjugated to 0, 1, 2, 3, or 4 pairs of cysteine residues on YSSFL.

In some embodiments, the ratio of effector agents to anti-CD46 antibody is 1:1, 2:1, 3:1, 4:1, 5:1, 6:1, 7:1 or 8:1. In some embodiments, the ratio of effector agents to anti-CD46 antibody is 2:1, 4:1, 6:1, or 8:1. In some embodiments, a ratio of said cytotic effector to said antibody that specifically binds CD46 or a CD46-binding fragment thereof is about 2:1, 4:1, 6:1, or 8:1. In some embodiments, the ratio of effector agents to anti-CD46 antibodies is about 4:1. In some embodiments, the average ratio of effector agents to anti-CD46 antibodies is about 3.7:1. In some embodiments, if the immunoconjugate comprises 2 or more effector agents, each effector agent is the same. In some embodiments, if the immunoconjugate comprises 2 or more effector agents, at least two effector agents are different. In some embodiments, the ratio of effector agents to anti-CD46 antibodies is about 4:1 and each effector agent is the same. In some embodiments, a ratio of said cytotic effector to said antibody that specifically binds CD46 or a CD46-binding fragment thereof is about 4:1.

### Exemplary Immunoconjugate

An exemplary immunoconjugate provided herein comprises an anti-CD46 YS5FL antibody linked to a monomethyl auristatin E (MMAE) effector agent via a maleimidocaproyl-valine-citrulline-para-amino benzyloxycarbonyl (mc-vc-PAB). In some embodiments, the ratio of MMAE to YSFL antibody is about 4:1.

In some embodiments, the immunoconjugate comprises the antibody conjugate below in Formula C, wherein the comprises heavy chain of SEQ ID NO: 169; and a light chain of SEQ ID NO: 170. This immunoconjugate is also referred to herein as FOR46 and comprises YS5FL antibody attached to MMAE through a mc-vc-PAB linker.

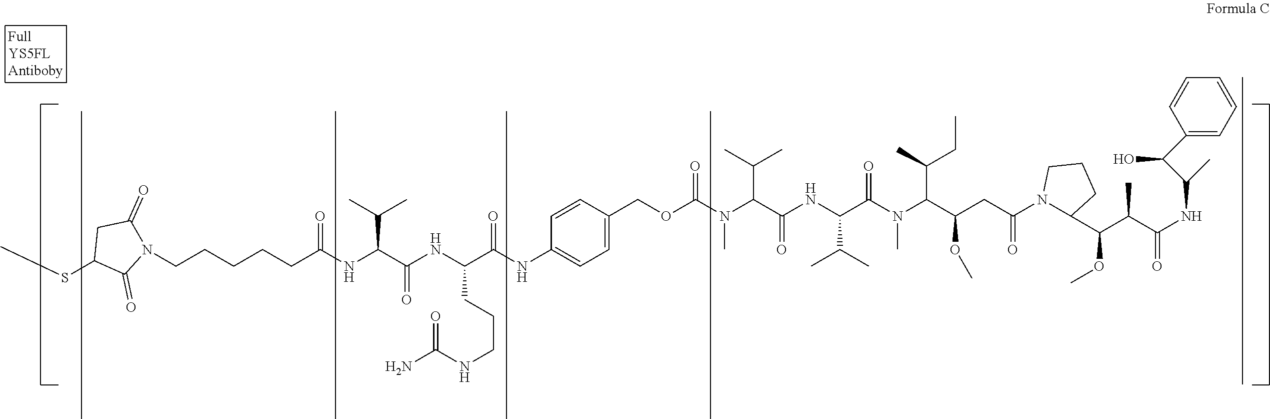

In some embodiments, an anti-CD46 immunoconjugate described herein is manufactured by a process comprising reduction or partial reduction of disulfide bonds of an immunoglobulin. In some embodiments, an anti-CD46 immunoconjugate described herein is manufactured by a process comprising reduction or partial reduction of interchain disulfide bonds of an immunoglobulin. In some embodiments, the reducing agent is dithiothreitol (DTT) or tris(2-carboxyethyl)phosphine (TCEP). In some embodiments, an effector-linker complex comprising a maleimide reactive group is conjugated to pairs of reduced cysteines of an immunoglobulin. In some embodiments, the effector-linker complex is mc-vc-PAB-MMAE.

In some embodiments, an effector-linker complex is conjugated at C219, C225, or C228 of a YS5FL heavy (SEQ ID NO: 169) or C214 of a YS5FL light chain (SEQ ID NO: 170), or any combination thereof. In some embodiments, the effector-linker complexes are conjugated to C219 of a YS5FL heavy chain and C214 of a YS5FL light chain. In some embodiments, an anti-CD46 immunoconjugate comprises two YS5FL heavy chains and two YS5FL light chains and effector-linker complexes are conjugated to C219 of a YS5FL first heavy chain, C214 of a first YS5FL light chain, C219 of a YS5FL second heavy chain, and C214 of a second YS5FL light chain. In some embodiments, an anti-CD46 immunoconjugate comprises two YS5FL heavy chains and an effector-linker complex is conjugated to C225 of a first YS5FL heavy chain and C225 of a second YS5FL heavy chain. In some embodiments, an anti-CD46 immunoconjugate comprises two YS5FL heavy chains and an effector-linker complex is conjugated to C228 of a first YS5FL heavy chain and C228 of a second YS5FL heavy chain. In some embodiments, an immunoconjugate comprises two, four, six, or eight effectors and the effectors are conjugated to any one, two, three, or four, respectively, of the following pairs of cysteines: C219 of HC1 and C214 of LC1; C219 of HC2 and C214 of LC2; C225 of HC1 and C225 of HC2; and C228 of HC1 and C228 of HC2.

### Immunoconjugate Binding to Target Cells and Activity on Target Cells

In some embodiments, an anti-CD46 antibody or immunoconjugate described herein binds to CD46 expressed on the surface of a target cell (e.g., a cancer cell) and is internalized by the cell. In some embodiments, the antibody or immunoconjugate is internalized into the target cell via macropinocytosis. In some embodiments, the antibody or immunoconjugate is targeted to a lysosome of the cell upon internalization. In some embodiments, the antibody or immunoconjugate induces internalization into the cell without crosslinking.

In some embodiments, an anti-CD46 antibody or immunoconjugate described herein mediates killing of a target cell (e.g., cancer cell) upon internalization. In some embodiments, the anti-CD46 antibody or immunoconjugate induces apoptosis of the target cell (e.g., cancer cell) upon internalization. In some embodiments, the anti-CD46 antibody or immunoconjugate inhibits cell division of the target cell (e.g., cancer cell) upon internalization. In some embodiments, the anti-CD46 antibody or immunoconjugate selectively inhibits cell division of cancer cells upon internalization and does not inhibit cell division of non-cancer cells upon internalization.

### Production of Antibodies or Antigen Binding Fragments Thereof

In some embodiments, antibodies (and antigen binding fragment thereof) are produced using any method known in the art to be useful for the synthesis of antibodies, in particular, by chemical synthesis or by recombinant expression techniques.

In some embodiments, an antibody (or antigen binding fragment thereof) is expressed recombinantly. In some embodiment, the nucleic acid encoding the antibody (or antigen binding fragment thereof) is assembled from chemically synthesized oligonucleotides. In some embodiments, a nucleic acid molecule encoding an antibody is generated from a suitable source (e.g., an antibody cDNA library, or cDNA library generated from any tissue or cells expressing the immunoglobulin) by PCR amplification using synthetic primers hybridizable to the 3′ and 5′ ends of the sequence or by cloning using an oligonucleotide probe specific for the particular gene sequence.

In some embodiments, an antibody (or antigen binding fragment thereof) is made by immunizing an animal, such as a mouse, to generate polyclonal or monoclonal antibodies.

In some embodiments, an expression vector comprising the nucleotide sequence of an antibody or the nucleotide sequence of an antibody is transferred to a host cell by conventional techniques (e.g., electroporation, liposomal transfection, and calcium phosphate precipitation), and the transfected cells are then cultured by conventional techniques to produce the antibody. In some embodiments, the expression of the antibody is regulated by a constitutive, an inducible or a tissue, specific promoter.

A variety of host-expression vector systems can be utilized to express an antibody (or antigen binding fragment thereof) described herein. These include, but are not limited to, microorganisms such as bacteria (e.g., E. coli and B. subtilis) transformed with recombinant bacteriophage DNA, plasmid DNA or cosmid DNA expression vectors containing an antibody or its binding fragment coding sequences; yeast (e.g., Saccharomyces Pichia) transformed with recombinant yeast expression vectors containing an antibody or its binding fragment coding sequences; insect cell systems infected with recombinant virus expression vectors (e.g., baculovirus) containing an antibody or its binding fragment coding sequences; plant cell systems infected with recombinant virus expression vectors (e.g., cauliflower mosaic virus (CaMV) and tobacco mosaic virus (TMV)) or transformed with recombinant plasmid expression vectors (e.g., Ti plasmid) containing an antibody or its binding fragment coding sequences; or mammalian cell systems (e.g., COS, CHO, BH, 293, 293T, 3T3 cells) harboring recombinant expression constructs containing promoters derived from the genome of mammalian cells (e.g., metallothionein promoter) or from mammalian viruses (e.g. the adenovirus late promoter; the vaccinia virus 7.5K promoter).

For long-term, high-yield production of recombinant proteins, stable expression may be preferred. In some embodiments, cell lines that stably express an antibody are made. Following the introduction of the foreign DNA, engineered cells are then allowed to grow for 1-2 days in an enriched media, and then are switched to a selective media. A selectable marker in the recombinant plasmid may be used to confer resistance to the selection.

In some embodiments, any method known in the art for purification of an antibody can be used, for example, by chromatography (e.g., ion exchange, affinity, particularly by affinity for the specific antigen after Protein A, and sizing column chromatography), centrifugation, differential solubility, or by any other standard technique for the purification of proteins.

### Expression Vectors

Vectors can include any suitable vector derived from either a eukaryotic or prokaryotic sources. In some cases, vectors are obtained from bacteria (e.g. E. coli), insects, yeast (e.g. Pichia pastoris), algae, or mammalian sources. Exemplary bacterial vectors include pACYC177, pASK75, pBAD vector series, pBADM vector series, pET vector series, pETM vector series, pGEX vector series, pHAT, pHAT2, pMal-c2, pMal-p2, pQE vector series, pRSET A, pRSET B, pRSET C, pTrcHis2 series, pZA31-Luc, pZE21-MCS-1, pFLAG ATS, pFLAG CTS, pFLAG MAC, pFLAG Shift-12c, pTAC-MAT-1, pFLAG CTC, or pTAC-MAT-2.

Exemplary insect vectors include pFastBacl, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

In some cases, yeast vectors include Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pA0815 Pichia vector, pFLD1 Pichi pastoris vector, pGAPZA,B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Exemplary algae vectors include pChlamy-4 vector or MCS vector.

Examples of mammalian vectors include transient expression vectors or stable expression vectors. Mammalian transient expression vectors may include pRK5, p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Mammalian stable expression vector may include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

In some instances, a cell-free system is a mixture of cytoplasmic and/or nuclear components from a cell and is used for in vitro nucleic acid synthesis. In some cases, a cell-free system utilizes either prokaryotic cell components or eukaryotic cell components. Sometimes, a nucleic acid synthesis is obtained in a cell-free system based on for example Drosophila cell, Xenopus egg, or HeLa cells. Exemplary cell-free systems include, but are not limited to, E. coli S30 Extract system, E. coli T7 S30 system, or PURExpress®.

### Host Cells

A host cell can be any suitable cell such as a naturally derived cell or a genetically modified cell. In some instances, a host cell is a production host cell. In some instances, a host cell is a eukaryotic cell. In other instances, a host cell is a prokaryotic cell. In some cases, a eukaryotic cell includes fungi (e.g., yeast cells), animal cell or plant cell. In some cases, a prokaryotic cell is a bacterial cell. Examples of bacterial cell include gram-positive bacteria or gram-negative bacteria. Sometimes the gram-negative bacteria is anaerobic, rod-shaped, or both.

In some instances, gram-positive bacteria include Actinobacteria, Firmicutes or Tenericutes. In some cases, gram-negative bacteria include Aquificae, Deinococcus-Thermus, Fibrobacteres-Chlorobi/Bacteroidetes (FCB group), Fusobacteria, Gemmatimonadetes, Nitrospirae, Planctomycetes- Verrucomicrobia/ Chlamydiae (PVC group), Proteobacteria, Spirochaetes or Synergistetes. Other bacteria can be Acidobacteria, Chloroflexi, Chrysiogenetes, Cyanobacteria, Deferribacteres, Dictyoglomi, Thermodesulfobacteria or Thermotogae. A bacterial cell can be Escherichia coli, Clostridium botulinum, or Coli bacilli.

Exemplary prokaryotic host cells include, but are not limited to, BL21, Mach1™, DH1OB™, TOP10, DH5α, DH10Bac™, OmniMax™, MegaX™, DH12S™, INV110, TOP1OF′, INVαF, TOP10/P3, ccdB Survival, PIR1, PIR2, Stb12™, Stb13™, or Stb14™.

In some instances, animal cells include a cell from a vertebrate or from an invertebrate. In some cases, an animal cell includes a cell from a marine invertebrate, fish, insects, amphibian, reptile, or mammal. In some cases, a fungus cell includes a yeast cell, such as brewer's yeast, baker's yeast, or wine yeast.

Fungi include ascomycetes such as yeast, mold, filamentous fungi, basidiomycetes, or zygomycetes. In some instances, yeast includes Ascomycota or Basidiomycota. In some cases, Ascomycota includes Saccharomycotina (true yeasts, e.g. Saccharomyces cerevisiae (baker's yeast)) or Taphrinomycotina (e.g. Schizosaccharomycetes (fission yeasts)). In some cases, Basidiomycota includes Agaricomycotina (e.g. Tremellomycetes) or Pucciniomycotina (e.g. Microbotryomycetes).

Exemplary yeast or filamentous fungi include, for example, the genus: Saccharomyces, Schizosaccharomyces, Candida, Pichia, Hansenula, Kluyveromyces, Zygosaccharomyces, Yarrowia, Trichosporon, Rhodosporidi, Aspergillus, Fusarium, or Trichoderma. Exemplary yeast or filamentous fungi include, for example, the species: Saccharomyces cerevisiae, Schizosaccharomyces pombe, Candida utilis, Candida boidini, Candida albicans, Candida tropicalis, Candida stellatoidea, Candida glabrata, Candida krusei, Candida parapsilosis, Candida guilliermondii, Candida viswanathii, Candida lusitaniae, Rhodotorula mucilaginosa, Pichia metanolica, Pichia angusta, Pichia pastoris, Pichia anomala, Hansenula polymorpha, Kluyveromyces lactis, Zygosaccharomyces rouxii, Yarrowia lipolytica, Trichosporon pullulans, Rhodosporidium toru-Aspergillus niger, Aspergillus nidulans, Aspergillus awamori, Aspergillus oryzae, Trichoderma reesei, Yarrowia lipolytica, Brettanomyces bruxellensis, Candida stellata, Schizosaccharomyces pombe, Torulaspora delbrueckii, Zygosaccharomyces bailii, Cryptococcus neoformans, Cryptococcus gattii, or Saccharomyces boulardii.

Exemplary yeast host cells include, but are not limited to, Pichia pastoris yeast strains such as GS115, KM71H, SMD1168, SMD1168H, and X-33; and Saccharomyces cerevisiae yeast strain such as INVSc1.

In some instances, additional animal cells include cells obtained from a mollusk, arthropod, annelid or sponge. In some cases, an additional animal cell is a mammalian cell, e.g., from a primate, ape, equine, bovine, porcine, canine, feline or rodent. In some cases, a rodent includes mouse, rat, hamster, gerbil, hamster, chinchilla, fancy rat, or guinea pig.

Exemplary mammalian host cells include, but are not limited to, 293A cell line, 293FT cell line, 293F cells, 293 H cells, CHO DG44 cells, CHO-S cells, CHO-K1 cells, FUT8 KO CHOK1, Expi293F™ cells, Flp-In™ T-REx™ 293 cell line, Flp-In™-293 cell line, Flp-In™-3T3 cell line, Flp-In™-BHK cell line, Flp-In™-CHO cell line, Flp-In™-CV-1 cell line, Flp-In™-Jurkat cell line, FreeStyle™ 293-F cells, FreeStyle™ CHO-S cells, GripTite™ 293 MSR cell line, GS-CHO cell line, HepaRG™ cells, T-REx™ Jurkat cell line, Per.C6 cells, T-REx™-293 cell line, T-REx™-CHO cell line, and T-REx™-HeLa cell line.

In some instances, a mammalian host cell is a stable cell line, or a cell line that has incorporated a genetic material of interest into its own genome and has the capability to express the product of the genetic material after many generations of cell division. In some cases, a mammalian host cell is a transient cell line, or a cell line that has not incorporated a genetic material of interest into its own genome and does not have the capability to express the product of the genetic material after many generations of cell division.

Exemplary insect host cells include, but are not limited to, Drosophila S2 cells, Sf9 cells, Sf21 cells, High FiveTM cells, and expresSF+® cells.

In some instances, plant cells include a cell from algae. Exemplary insect cell lines include, but are not limited to, strains from Chlamydomonas reinhardtii 137c, or Synechococcus elongatus PPC 7942.

### Therapeutic Methods

Disclosed herein, in some embodiments, are methods of treating cancer in a subject in need thereof. Some embodiments include administering to said subject a first anti-cancer agent. In some embodiments, the first anti-cancer agent increases CD46 expression on a cancer cell. In some embodiments, the first anti-cancer agent enhances the efficacy of the second anti-cancer agent. Some embodiments include administering to said subject a second anti-cancer agent. In some embodiments, the second anti-cancer agent comprises an antibody that specifically binds CD46 or a CD46-binding fragment thereof. In some embodiments, the second anti-cancer agent comprises an anti-CD46 antibody conjugated to a cytotoxic effector. In some embodiments, the combination of the first and second anti-cancer agents is synergistic in treating cancer in the subject.

In some embodiments, a first dose of said first anti-cancer agent is administered before a first dose of said second anti-cancer agent. For example, the first anti-cancer agent may increase CD46 expression, and then the second anti-cancer agent may be administered when CD46 expression is higher.

In some embodiments, the cancer comprises cancer cells with no CD46 expression that is induced upon exposure to the first anti-cancer agent. In some embodiments, the cancer comprises cancer cells with low CD46 expression that is increased upon exposure to the first anti-cancer agent. In some embodiments, the cancer comprises cancer cells with average CD46 expression that is increased upon exposure to the first anti-cancer agent. In some embodiments, the cancer comprises cancer cells with high CD46 expression that is increased further upon exposure to the first anti-cancer agent. Thereafter, upon administration of the second anti-cancer agent, the second anti-cancer agent may bind the CD46.

In some embodiments, the cancer comprises breast cancer. In some embodiments, the cancer comprises a cancer described in Maciejczyk, CD46 Expression is an unfavorable prognostic factor in breast cancer cases, Appl Immunohistochem Mol Morphol. 2011 Dec;19(6):540-6, the contents of which are entirely incorporated herein by reference. In some embodiments, the cancer comprises ovarian cancer. In some embodiments, the cancer comprises a cancer described in Surowiak, CD46 expression is indicative of shorter revival-free survival for ovarian cancer patients, Anticancer Res. Nov-Dec 2006;26(6C):4943-8, the contents of which are entirely incorporated herein by reference. In some embodiments, the cancer comprises renal cancer. In some embodiments, the cancer comprises a cancer described in Blok, A Possible Role of CD46 for the Protection In Vivo of Human Renal Tumor Cells from Complement-Mediated Damage, Lab Invest. 2000 Mar;80(3):335-44, the contents of which are entirely incorporated herein by reference. In some embodiments, the cancer comprises cervical cancer. In some embodiments, the cancer comprises colorectal cancer. In some embodiments, the cancer comprises adenocarcinoma. In some embodiments, the cancer comprises a neuroendocrine cancer.

In some embodiments, said cancer comprises ovarian cancer, colorectal cancer, breast cancer, lung cancer, prostate cancer, kidney cancer, pancreatic cancer, mesothelioma, lymphoma, liver cancer, urothelial cancer, stomach cancer, multiple myeloma, glioblastoma multiforme, glioma, neuroblastoma, or cervical cancer. In some embodiments, said cancer is not a prostate cancer or a multiple myeloma. In some embodiments, said cancer comprises ovarian cancer, colorectal cancer, breast cancer, lung cancer, kidney cancer, pancreatic cancer, mesothelioma, lymphoma, liver cancer, urothelial cancer, stomach cancer, glioblastoma multiforme, glioma, neuroblastoma, or cervical cancer.

In some embodiments, the cancer is multiple myeloma. In some embodiments, the cancer is relapsing multiple myeloma. In some embodiments, the cancer is remitting multiple myeloma. In some embodiments, the cancer is relapsing or remitting multiple myeloma. In some embodiments, the cancer is not multiple myeloma. In some embodiments, the cancer is not relapsing multiple myeloma. In some embodiments, the cancer is not remitting multiple myeloma. In some embodiments, the cancer is not relapsing or remitting multiple myeloma.

In some embodiments, the cancer is prostate cancer. In some embodiments, the cancer is castration resistant prostate cancer. In some embodiments, the cancer is metastatic prostate cancer. In some embodiments, the cancer is not prostate cancer. In some embodiments, the cancer is not castration resistant prostate cancer. In some embodiments, the cancer is not metastatic prostate cancer.

In some embodiments, the cancer comprises a metastatic cancer. In some embodiments, the cancer comprises a localized cancer. In some embodiments, the cancer comprises a benign cancer.

In one aspect, provided herein are methods of treating cancer by administering a first anti-cancer agent that increases CD46 expression in a cancer cell, and a second anti-cancer agent (e.g. an anti-CD46 antibody or immunoconjugate described herein).

In one aspect, provided herein are a first anti-cancer agent that increases CD46 expression in a cancer cell, and a second anti-cancer agent (e.g. an anti-CD46 antibody or immunoconjugate described herein), for use as a medicament. In one aspect, provided herein are a first anti-cancer agent that increases CD46 expression in a cancer cell, and a second anti-cancer agent (e.g. an anti-CD46 antibody or immunoconjugate described herein), for use in treating a disease, in particular for use in the treatment of cancer. In one aspect, provided herein are a first anti-cancer agent that increases CD46 expression in a cancer cell, and a second anti-cancer agent (e.g. an anti-CD46 antibody or immunoconjugate described herein), for use in a method of treating cancer. In one aspect, provided herein are a first anti-cancer agent that increases CD46 expression in a cancer cell, and a second anti-cancer agent (e.g. an anti-CD46 antibody or immunoconjugate described herein), for use in the treatment of a disease in an individual in need thereof. In one aspect, provided herein are a first anti-cancer agent that increases CD46 expression in a cancer cell, and a second anti-cancer agent (e.g. an anti-CD46 antibody or immunoconjugate described herein), for use in a method of treating an individual having cancer comprising administering to the individual a therapeutically effective amount of the first and second anti-cancer agents described herein. In one aspect, provided herein are a first anti-cancer agent that increases CD46 expression in a cancer cell, and a second anti-cancer agent (e.g. an anti-CD46 antibody or immunoconjugate described herein), in the manufacture or preparation of a medicament for the treatment of a disease in an individual in need thereof. In one aspect, provided herein are the medicament is for use in a method of treating a cancer comprising administering to an individual having cancer a therapeutically effective amount of the medicament.

In some embodiments, said subject is a mammal. In some embodiments, said subject is a human. In some embodiments, said subject is a non-human mammal. In some embodiments, said subject is a primate. In some embodiments, said subject is a non-human primate.

In some embodiments, said first anti-cancer agent is administered as part of a first pharmaceutical composition. In some embodiments, said second anti-cancer agent is administered as part of said first pharmaceutical composition. In some embodiments, said second anti-cancer agent is administered as part of a second pharmaceutical composition.

### Additional Therapeutic Methods

In some embodiments, disclosed herein are methods of treating a subject with cancer comprising exposing the cancer to radiation followed by treating the subject with an anti-cancer agent comprising an antibody that specifically binds CD46 or a CD46-binding fragment thereof. In some embodiments, disclosed herein are methods of treating a subject with cancer comprising exposing the cancer to radiation, determining that CD46 expression in the cancer has increased, and then treating the subject with an anti-cancer agent comprising an antibody that specifically binds CD46 or a CD46-binding fragment thereof.

Disclosed herein, in some embodiments, are methods of treating cancer in a subject in need thereof. Some embodiments include administering to said subject a first anti-cancer agent. In some embodiments, the first anti-cancer agent increases CD46 expression on a cancer cell. In some embodiments, the first anti-cancer agent enhances the efficacy of the second anti-cancer agent. Some embodiments include administering to said subject a second anti-cancer agent. In some embodiments, the second anti-cancer agent comprises an antibody that specifically binds CD46 or a CD46-binding fragment thereof In some embodiments, the second anti-cancer agent comprises an anti-CD46 antibody conjugated to a cytotoxic effector. In some embodiments, the combination of the first and second anti-cancer agents is synergistic in treating cancer in the subject. In some embodiments, the first anti-cancer agent is anastrozole, temozolomide, regorafenib, gemcitabine, capecitabine, trastuzumab, tamoxifen, docetaxel, cisplatin, doxorubicin, leucovorin, cabazitaxel, durvalumab, oxaliplatin, fluorouracil, irinotecan, oxaliplatin, carboplatin, bevacizumab, cetuximab, prednisone, ifosfamide, aflibercept, pembrolizumab, or paclitaxel. In some embodiments, the cancer is breast cancer and the first anti-cancer agent is anastrozole. In some embodiments, the cancer is glioblastoma and the first anti-cancer agent is temozolomide. In some embodiments, the cancer is colorectal cancer and the first anti-cancer agent is regorafenib. In some embodiments, the cancer is ovarian cancer and the first anti-cancer agent is gemcitabine. In some embodiments, the cancer is pancreatic cancer and the first anti-cancer agent is gemcitabine. In some embodiments, the cancer is pancreatic cancer and the first anti-cancer agent is capecitabine. In some embodiments, the cancer is breast cancer and the first anti-cancer agent is trastuzumab. In some embodiments, the cancer is breast cancer and the first anti-cancer agent is tamoxifen. In some embodiments, the cancer is breast and the first anti-cancer agent is docetaxel. In some embodiments, the cancer is bladder cancer and the first anti-cancer agent is cisplatin. In some embodiments, the cancer is sarcoma and the first anti-cancer agent is doxorubicin. In some embodiments, the cancer is biliary cancer and the first anti-cancer agent is cisplatin. In some embodiments, the cancer is pancreatic cancer and the first anti-cancer agent is cisplatin. In some embodiments, the cancer is breast cancer and the first anti-cancer agent is doxorubicin. In some embodiments, the cancer is pancreatic cancer and the first anti-cancer agent is gemcitabine. In some embodiments, the cancer is pancreatic cancer and the first anti-cancer agent is leucovorin. In some embodiments, the cancer is prostate cancer and the first anti-cancer agent is cabazitaxel. In some embodiments, the cancer is colorectal cancer and the first anti-cancer agent is capecitabine. In some embodiments, the cancer is non-small cell lung cancer and the first anti-cancer agent is durvalumab. In some embodiments, the cancer is gastric cancer and the first anti-cancer agent is oxaliplatin. In some embodiments, the cancer is pancreatic cancer and the first anti-cancer agent is fluorouracil. In some embodiments, the cancer is colorectal cancer and the first anti-cancer agent is irinotecan. In some embodiments, the cancer is pancreatic cancer and the first anti-cancer agent is oxaliplatin. In some embodiments, the cancer is endometrial cancer and the first anti-cancer agent is carboplatin. In some embodiments, the cancer is glioblastoma cancer and the first anti-cancer agent is bevacizumab. In some embodiments, the cancer is glioblastoma cancer and the first anti-cancer agent is bevacizumab. In some embodiments, the cancer is colorectal cancer and the first anti-cancer agent is cetuximab. In some embodiments, the cancer is prostate cancer and the first anti-cancer agent is prednisone. In some embodiments, the cancer is sarcoma and the first anti-cancer agent is ifosfamide. In some embodiments, the cancer is endometrial cancer and the first anti-cancer agent is paclitaxel. In some embodiments, the cancer is breast cancer and the first anti-cancer agent is gemcitabine. In some embodiments, the cancer is esophageal cancer and the first anti-cancer agent is paclitaxel. In some embodiments, the cancer is breast cancer and the first anti-cancer agent is zoledronic acid. In some embodiments, the cancer is breast cancer and the first anti-cancer agent is denosumab. In some embodiments, the cancer is breast cancer and the first anti-cancer agent is bevacizumab. In some embodiments, the cancer is breast cancer and the first anti-cancer agent is methylprednisolone. In some embodiments, the cancer is colorectal cancer and the first anti-cancer agent is panitumumab. In some embodiments, the cancer is colorectal cancer and the first anti-cancer agent is leucovorin. In some embodiments, the cancer is colorectal cancer and the first anti-cancer agent is carboplatin. In some embodiments, the cancer is colorectal cancer and the first anti-cancer agent is pembrolizumab. In some embodiments, the cancer is colorectal cancer and the first anti-cancer agent is pegfilgrastim. In some embodiments, the cancer is colorectal cancer and the first anti-cancer agent is metformin. In some embodiments, the cancer is endometrial cancer and the first anti-cancer agent is cisplatin. In some embodiments, the cancer is glioblastoma cancer and the first anti-cancer agent is temozolomide. In some embodiments, the cancer is head and neck squamous cell carcinoma and the first anti-cancer agent is cetuximab. In some embodiments, the cancer is head and neck squamous cell carcinoma and the first anti-cancer agent is cisplatin. In some embodiments, the cancer is sarcoma and the first anti-cancer agent is etoposide. In some embodiments, the cancer is prostate cancer and the first anti-cancer agent is abiraterone. In some embodiments, the cancer is sarcoma and the first anti-cancer agent is doxorubicin liposome. In some embodiments, the cancer is esophageal cancer and the first anti-cancer agent is trastuzumab. In some embodiments, the cancer is colorectal cancer and the first anti-cancer agent is mitomycin. In some embodiments, the cancer is sarcoma and the first anti-cancer agent is cisplatin. In some embodiments, the cancer is endocrine tumor and the first anti-cancer agent is carboplatin. In some embodiments, the cancer is breast cancer and the first anti-cancer agent is endocrine therapy. In some embodiments, the cancer is colorectal cancer and the first anti-cancer agent is aflibercept. In some embodiments, the cancer is head and neck squamous cell carcinoma and the first anti-cancer agent is pembrolizumab.

In some embodiments, disclosed herein are methods of treating a subject in need thereof with an anti-cancer agent comprising an antibody that specifically binds CD46 or a CD46-binding fragment thereof, wherein the subject has not been previously treated with fluorouracil, leuprolide, doxorubicin, pemetrexed, oxaliplatin, bicalutamide, gemcitabine, osimertinib, olaparib, leucovorin, trastazumab, eribulin, cisplatin, bevacizumab, palbociclib, paclitaxel, pembrolizumab, fulvestrant, atezolizumab, or dexamethasone. In some embodiments, a subject with cancer is treated with an anti-cancer agent comprising an antibody that specifically binds CD46 or a CD46-binding fragment thereof before being treated with an agent that reduces CD46 expression. In some embodiments, a subject with esophageal cancer is treated with an anti-cancer agent comprising an antibody that specifically binds CD46 or a CD46-binding fragment thereof before being treated with fluorouracil. In some embodiments, a subject with non small cell lung cancer is treated with an anti-cancer agent comprising an antibody that specifically binds CD46 or a CD46-binding fragment thereof before being treated with pemetrexed. In some embodiments, a subject with non clear cell renal carcinoma is treated with an anti-cancer agent comprising an antibody that specifically binds CD46 or a CD46-binding fragment thereof before being treated with axitinib.

In some embodiments, disclosed herein are methods of treating a subject with cancer comprising treating the subject with a first anti-cancer agent, determining that CD46 expression has increased in the cancer, and then treating the subject with a second anti-cancer agent comprising an antibody that specifically binds CD46 or a CD46-binding fragment thereof In some embodiments, determining that CD46 expression has increased in the cancer comprises comparing a CD46 expression level in a biopsy collected before treatment with the first anti-cancer agent to a CD46 expression level in a biopsy collected after treatment with the first anti-cancer agent. In some embodiments, the before biopsy is collected about 2, 3, 4, 5, 6, 7, 8, 9, 10, 11, or 12 weeks before the after biopsy. In some embodiments, the before biopsy is collected about 1, 2, 3, 4, 5, or 6 months before the after biopsy. In some embodiments, the cancer comprises multiple myeloma, relapsing multiple myeloma or remitting multiple myeloma. In some embodiments, the cancer comprises prostate cancer, castration resistant prostate cancer, and/or metastatic prostate cancer. In some cases, the cancer comprises ovarian cancer, colorectal cancer, breast cancer, lung cancer, prostate cancer, kidney cancer, pancreatic cancer, mesothelioma, lymphoma, liver cancer, urothelial cancer, stomach cancer, multiple myeloma, glioblastoma multiforme, glioma, neuroblastoma, or cervical cancer. In some embodiments, the first anti-cancer agent is abiraterone, alpelisib, anastrozole, atezolizumab, bevacizumab, bicalutamide, cabazitaxel, capecitabine, carboplatin, cetuximab, cisplatin, cyclophosphamide, dexamethasone, docetaxel, doxorubicin, doxorubicin, liposome, durvalumab, enzalutamide, eribulin, exemestane, fluorouracil, fulvestrant, gemcitabine, ifosfamide, ipilimumab, irinotecan, letrozole, leucovorin, leuprolide, nivolumab, olaparib, osimertinib, oxaliplatin, paclitaxel, protein-bound paclitaxel, palbociclib, panitumumab, pembrolizumab, pemetrexed, prednisone, regorafenib, tamoxifen, temozolomide, or trastuzumab.

### Dosing and Administration

For use in therapeutic methods, a first anti-cancer agent that increases CD46 on cancer cells, or a second anti-cancer agent (e.g. anti-CD46 antibodies or immunoconjugates described herein) can be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In some embodiments, said first anti-cancer agent and/or said second anti-cancer agent is administered to said human subject orally, nasally, rectally, intraperitoneally, subcutaneously, transcutaneously, intramuscularly, or intravenously. In some embodiments, said first anti-cancer agent is administered to said human subject orally, nasally, rectally, intraperitoneally, subcutaneously, transcutaneously, intramuscularly, or intravenously. In some embodiments, said second anti-cancer agent is administered to said human subject orally, nasally, rectally, intraperitoneally, subcutaneously, transcutaneously, intramuscularly, or intravenously. In some embodiments, said first anti-cancer agent and/or said second anti-cancer agent is administered to said human subject via intravenous infusion. In some embodiments, said first anti-cancer agent is administered to said human subject via intravenous infusion. In some embodiments, said second anti-cancer agent is administered to said human subject via intravenous infusion.

In some embodiments, the first anti-cancer agent is administered to a human subject every 7 days, every 14 days, every 18 days, every 21 days, or every 30 days. In some embodiments, the second anti-cancer agent is administered to a human subject every 7 days, every 14 days, every 18 days, every 21 days, or every 30 days. In some embodiments, the second anti-cancer agent is administered to a human subject every 21 days.

In some embodiments, the first or second anti-cancer agent is administered to a human subject at a dose from about 1.0 to 5.0 mg/kg. In some embodiments, the antibody or immunoconjugate is administered to a human subject at a dose from about 1.0 to 5.0 mg/kg. In some embodiments, the antibody or immunoconjugate to at a dose from about 1.0 to 4.5 mg/kg, 1.0 to 4.0 mg/kg, 1.0 to 3.5 mg/kg, 1.0 to 3.0 mg/kg, 1.0 to 2.5 mg/kg, 1.5 to 4.5 mg/kg, 1.5 to 4.0 mg/kg, 1.5 to 3.5 mg/kg, 1.5 to 3.0 mg/kg, 1.5 to 2.5 mg/kg, 1.5 to 2.0 mg/kg, 1.8 to 4.5, 1.8 to 4.0, 1.8 to 3.5, 1.8 to 3.0, 1.8 to 2.5, or 1.8 to 2.0. In some embodiments, the antibody or immunoconjugate is administered to a human subject at a dose from about 1.5 to 2.5 mg/kg. In some embodiments, the first or second anti-cancer agent is administered to the subject at dose lower than an aforementioned dose due to the synergistic effects of the first and second anti-cancer agents.

In some embodiments, the second anti-cancer agent (e.g. antibody or immunoconjugate) is administered to a human subject at a dose of about 1.0, 1.1, 1.2, 1.3, 1.4, 1.5, 1.6, 1.7, 1.8 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9 or 4.0 mg/kg. In some embodiments, the antibody or immunoconjugate is administered to a human subject at a dose of about 1.8, 2.4, or 3.2 mg/kg. In some embodiments, the antibody or immunoconjugate is administered to a human subject at a dose of about 1.8 mg/kg. In some embodiments, the antibody or immunoconjugate is administered to a human subject at a dose of about 2.4 mg/kg. In some embodiments, the antibody or immunoconjugate is administered to a human subject at a dose of about 3.2 mg/kg. In some embodiments, the antibody or immunoconjugate is administered to a human subject at a dose 1.5 mg/kg. In some embodiments, the antibody or immunoconjugate is administered to a human subject at a dose 2.5 mg/kg. In some embodiments, the antibody or immunoconjugate is administered to a human subject at a dose 3.0 mg/kg.

In some embodiments, said first anti-cancer agent and/or second anti-cancer agent is administered in an effective amount. In some embodiments, said first anti-cancer is administered in an effective amount. In some embodiments, said second anti-cancer agent is administered in an effective amount. In some embodiments, said effective amount of the second anti-cancer agent comprises a dose from about 1.0 to 5.0 mg/kg. In some embodiments, said dose is about 1.2 mg/kg. In some embodiments, said dose is about 1.8 mg/kg. In some embodiments, said dose is about 2.4 mg/kg. In some embodiments, said dose is about 3.2 mg/kg. In some embodiments, said dose is administered every 2-4 weeks. In some embodiments, said dose is administered about every 3 weeks.

In some embodiments, said effective amount of the first anti-cancer agent increases a response of the cancer cell to the second anti-cancer agent. In some embodiments, said increased CD46 expression on the surface of the cancer cell is relative to a control measurement or relative to a baseline measurement. In some embodiments, said increased CD46 expression on the surface of the cancer cell is relative to a control measurement. In some embodiments, the control measurement includes an average measurement, such as an average CD46 expression level in cancer samples from a population of subjects. In some embodiments, said increased CD46 expression on the surface of the cancer cell is relative to a baseline measurement. In some embodiments, the baseline measurement includes an average measurement, such as an average CD46 expression level in cancer samples from a population of subjects. In some embodiments, the baseline measurement includes a baseline measurement in the subject being treated, but before treatment (e.g. before an administration of the first or second anti-cancer agent).

In some embodiments, said first anti-cancer agent enhances an antibody-dependent cellular cytotoxicty activity of the second anti-cancer agent on the cancer cell. In some embodiments, said effective amount of the first anti-cancer agent or of the second anti-cancer agent is lower than an effective amount in a method not including administration of both the first anti-cancer agent and second anti-cancer agent. In some embodiments, said effective amount of the first anti-cancer agent is lower than an effective amount in a method not including administration of both the first anti-cancer agent and second anti-cancer agent. In some embodiments, said effective amount of the second anti-cancer agent is lower than an effective amount in a method not including administration of both the first anti-cancer agent and second anti-cancer agent.

### Pharmaceutical Compositions and Formulations

In a further aspect, the invention provides pharmaceutical compositions comprising an anti-CD46 antibody or immunoconjugate described herein, or comprising an anti-cancer agent that increases CD46 on a cancer cell or otherwise enhances the efficacy of an anti-cancer agent comprising a CD46-binding domain, e.g., for use in any of the above therapeutic methods. In one embodiment, a pharmaceutical composition comprises a first anti-cancer agent that increases CD46 on a cancer cell provided herein, a second anti-cancer agent comprising an anti-CD46 antibody or binding fragment thereof, and at least one pharmaceutically acceptable excipient. In one embodiment, a first pharmaceutical composition comprises an anti-cancer agent that increases CD46 on a cancer cell provided herein and at least one pharmaceutically acceptable excipient. In one embodiment, a second pharmaceutical composition comprises an anti-CD46 antibody or immunoconjugate provided herein and at least one pharmaceutically acceptable excipient. The preparation of such pharmaceutical compositions will be known to those of skill in the art in light of the present disclosure, as exemplified by Remington's Pharmaceutical Sciences, 18th Ed. Mack Printing Company, 1990, incorporated by reference herein.

Some embodiments describe a pharmaceutical composition (e.g. including said first anti-cancer agent or said second anti-cancer agent, or both). Reference to the pharmaceutical composition may include a first pharmaceutical composition or a second pharmaceutical composition.

In some embodiments, the pharmaceutical composition comprises a buffer. In some embodiments, the buffer comprises histidine. In some embodiments, the pharmaceutical composition comprises from about 10 to 40 mM, 10 to 30 mM, or 10 to 20 mM histidine buffer. In some embodiments, the pharmaceutical composition comprises about 10 mM, 15 mM, 20 mM, 25 mM, 30 mM, 35 mM, or 40 mM histidine buffer. In some embodiments, the pharmaceutical composition comprises about 20 mM histidine buffer. In some embodiments, said first and/or second pharmaceutical composition comprises from about 10 to 30 mM histidine buffer. In some embodiments, said first pharmaceutical composition comprises from about 10 to 30 mM histidine buffer. In some embodiments, said second pharmaceutical composition comprises from about 10 to 30 mM histidine buffer.

In some embodiments, the pharmaceutical composition comprises a cryoprotectant. In some embodiments, said first and/or second pharmaceutical composition comprises a cryoprotectant. In some embodiments, said first pharmaceutical composition comprises a cryoprotectant. In some embodiments, said second pharmaceutical composition comprises a cryoprotectant. In some embodiments, the cryoprotectant comprises a saccharide. In some embodiments, said cryoprotectant is a saccharide, sucrose, or trehalose. In some embodiments, the cryoprotectant comprises sucrose or trehalose. In some embodiments, the cryoprotectant comprises sucrose. In some embodiments, the pharmaceutical composition comprises from about 4% to 12%, 4% to 11%, 4% to 10%, 4% to 9%, 4% to 8%, 5% to 12%, 5% to 11%, 5% to 10%, 5% to 9%, 5% to 8%, 6% to 12%, 6% to 11%, 6% to 10%, 6% to 9%, 6% to 8%, 7% to 12%, 7% to 11%, 7% to 10%, 7% to 9%, or 7% to 8% sucrose. In some embodiments, the pharmaceutical composition comprises about 8% sucrose.

In some embodiments, the pharmaceutical composition comprises a stabilizing agent. In some embodiments, said first and/or second pharmaceutical composition comprises a stabilizing agent. In some embodiments, said first pharmaceutical composition comprises a stabilizing agent. In some embodiments, said second pharmaceutical composition comprises a stabilizing agent. In some embodiments, the stabilizing agent prevents denaturation of said recombinant antibody, prevents aggregation of said immunoconjugates, or both. In some embodiments, said stabilizing agent prevents denaturation of said first anti-cancer agent, prevents aggregation of said second anti-cancer agent, or both. In some embodiments, the stabilizing agent is a polysorbate. In some embodiments, the stabilizing agent is polysorbate 20. In some embodiments, the stabilizing agent is polysorbate 80. In some embodiments, the pharmaceutical composition comprises a polysorbate (e.g., polysorbate 80) from about 0.001% to 0.1%, 0.001% to 0.05%, 0.001% to 0.04%, 0.001% to 0.03%, 0.001% to 0.02%, or 0.001% to 0.01%. In some embodiments, the pharmaceutical composition comprises a polysorbate (e.g., polysorbate 80) at about 0.01%, 0.02%, 0.03%, 0.04%, 0.05%, 0.06%, 0.07%, 0.08%, 0.09%, or 0.1%. In some embodiments, the pharmaceutical composition comprises a polysorbate (e.g., polysorbate 80) at about 0.01%.

In some embodiments, the pharmaceutical composition has a pH of from about 5.0 to 7.0. In some embodiments, said first and/or second pharmaceutical composition comprises a pH from about 5.0 to 7.0. In some embodiments, said first pharmaceutical composition comprises a pH from about 5.0 to 7.0. In some embodiments, said second pharmaceutical composition comprises a pH from about 5.0 to 7.0. In some embodiments, the pharmaceutical composition has a pH of about 5.0, 5.5, 6.0, 6.5, 7.0, or 7.5. In some embodiments, the pharmaceutical composition has a pH of about 6.0.

In some embodiments, a pharmaceutical composition comprises second anti-cancer agent (e.g. an anti-CD46 antibody or immunoconjugate described herein) at a concentration from about 5.0 mg/ml to 15.0 mg/ml, 5.0 mg/ml to 14.0 mg/ml, 5.0 mg/ml to 13.0 mg/ml, 5.0 mg/ml to 12.0 mg/ml, 5.0 mg/ml to 11.0 mg/ml, 5.0 mg/ml to 10.0 mg/ml, 6.0 mg/ml to 15.0 mg/ml, 7.0 mg/ml to 15.0 mg/ml, 8.0 mg/ml to 15.0 mg/ml, 9.0 mg/ml to 15.0 mg/ml, or 10.0 mg/ml to 15.0 mg/ml. In some embodiments, pharmaceutical composition comprises an anti-CD46 antibody or immunoconjugate described herein at a concentration of about 5.0 mg/ml, 6.0 mg/ml, 7.0 mg/ml, 8.0 mg/ml, 9.0 mg/ml, 10.0 mg/ml, 11.0 mg/ml, 12.0 mg/ml, 13.0 mg/ml, 14.0 mg/ml, or 15.0 mg/ml. In some embodiments, the pharmaceutical composition comprises an anti-CD46 antibody or immunoconjugate described herein at a concentration of about 5.0 mg/ml±1.0 mg/mL, 6.0 mg/ml±1.0 mg/mL, 7.0 mg/ml±1.0 mg/mL, 8.0 mg/ml±1.0 mg/mL, 9.0 mg/ml±1.0 mg/mL, 10.0 mg/ml±1.0 mg/mL, 11.0 mg/ml±1.0 mg/mL, 12.0 mg/ml±1.0 mg/mL, 13.0 mg/ml±1.0 mg/mL, 14.0 mg/ml±1.0 mg/mL, or 15.0 mg/ml±1.0 mg/mL. In some embodiments, the pharmaceutical composition comprises an anti-CD46 antibody or immunoconjugate described herein at a concentration of about 10.0 mg/ml±1.0 mg/mL.

### Exemplary Formulation

An exemplary formulation of an anti-CD46 antibody or immunoconjugate described herein comprises about an anti-CD46 antibody or immunoconjugate described herein at a concentration of about 10.0 mg/ml±1.0 mg/mL; about 20 mM histidine buffer, about 8.0% sucrose, about 0.01% polysorbate 80, pH 6.0. The anti-CD46 antibody or immunoconjugate may be formulated together with the first anti-cancer agent, or may be administered separately with the first anti-cancer agent.

### Articles of Manufacture

In another aspect of the invention, an article of manufacture containing materials useful for the treatment of cancers described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating the condition and may have a sterile access port (for example the container may be an intravenous solution bag or a vial having a stopper that is pierceable by a hypodermic injection needle).

The label or package insert indicates that the composition is used for treating the condition of choice. Moreover, the article of manufacture may comprise (a) a first container with a composition contained therein, wherein the composition comprises the bispecific antibody of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition.

Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

Definitions

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. The section headings used herein are for organizational purposes only and are not to be construed as limiting the subject matter described. In this application, the use of the singular includes the plural unless specifically stated otherwise. It is noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error.

The terms “antibody” and “immunoglobulin” are used interchangeably herein and are used in the broadest sense and covers fully assembled antibodies, antibody fragments that can bind antigen, for example, Fab, F(ab′)2, Fv, single chain antibodies (scFv), diabodies, antibody chimeras, hybrid antibodies, bispecific antibodies, and the like.

The terms “monoclonal antibody” and “mAb” are used interchangeably herein and refer to an antibody obtained from a substantially homogeneous population of antibodies, i.e., the individual antibodies of the population are identical except for possible naturally occurring mutations that may be present in minor amounts.

The terms “native antibodies” and “native immunoglobulins” are heterotetrameric glycoproteins of about 150,000 Daltons, composed of two identical light (L) chains and two identical heavy (H) chains. Each light chain is linked to a heavy chain by one covalent disulfide bond, while the number of disulfide linkages varies among the heavy chains of different immunoglobulin isotypes. Each heavy and light chain also has regularly spaced intrachain disulfide bridges. Each heavy chain has at one end a variable domain (VH) followed by a number of constant domains. Each light chain has a variable domain at one end (VL) and a constant domain at its other end; the constant domain of the light chain is aligned with the first constant domain of the heavy chain, and the light chain variable domain is aligned with the variable domain of the heavy chain. Particular amino acid residues are believed to form an interface between the light and heavy-chain variable domains.

The term “hypervariable region,” as used herein, refers to the amino acid residues of an antibody that are responsible for antigen-binding. The hypervariable region comprises amino acid residues from a “complementarily determining region” or “CDR” (i.e., residues 24-34 (L1), 50-56 (L2), and 89-97 (L3) in the light-chain variable domain and 31-35 (H1), 50-65 (H2), and 95-102 (H3) in the heavy-chain variable domain; Kabat et al. (1991) Sequences of Proteins of Immunological Interest Fifth Edition, U.S. Department of Health and Human Services, NIH Publication No. 91-3242 (referred to herein as “Kabat et al”) and/or those residues from a “hypervariable loop” (i.e., residues 26-32 (L1), 50-52 (L2), and 91-96 (L3) in the light-chain variable domain and (H1), 53-55 (H2), and 96-101 (13) in the heavy chain variable domain; Chothia and Lesk, (1987) J. Mol. Biol., 196:901-917). “Framework” or “FR” residues are those variable domain residues other than the hypervariable region residues, as herein deemed.

In some instances, the CDRs of an antibody is determined according to (i) the Kabat numbering system Kabat et al. (1991) Sequences of Proteins of Immunological Interest Fifth Edition, U.S. Department of Health and Human Services, NIH Publication No. 91-3242; or (ii) the Chothia numbering scheme, which will be referred to herein as the “Chothia CDRs” (see, e.g., Chothia and Lesk, 1987, J. Mol. Biol., 196:901-917; Al-Lazikani et al., 1997, J. Mol. Biol., 273 :927-948; Chothia et al., 1992, J. Mol. Biol., 227:799-817; Tramontano A et al., 1990, J. Mol. Biol. 215(1): 175-82; and U.S. Pat. No. 7,709,226); or (iii) the ImMunoGeneTics (IMGT) numbering system, for example, as described in Lefranc, M.-P., 1999, The Immunologist, 7: 132-136 and Lefranc, M.-P. et al, 1999, Nucleic Acids Res., 27:209-212 (“IMGT CDRs”); or (iv) MacCallum et al, 1996, J. Mol. Biol., 262:732-745. See also, e.g., Martin, A., “Protein Sequence and Structure Analysis of Antibody Variable Domains,” in Antibody Engineering, Kontermann and Diibel, eds., Chapter 31, pp. 422-439, Springer-Verlag, Berlin (2001).

With respect to the Kabat numbering system, CDRs within an antibody heavy chain molecule are typically present at amino acid positions 31 to 35, which optionally can include one or two additional amino acids, following 35 (referred to in the Kabat numbering scheme as 35 A and 35B) (CDR1), amino acid positions 50 to 65 (CDR2), and amino acid positions 95 to 102 (CDR3). Using the Kabat numbering system, CDRs within an antibody light chain molecule are typically present at amino acid positions 24 to 34 (CDR1), amino acid positions 50 to 56 (CDR2), and amino acid positions 89 to 97 (CDR3). As is well known to those of skill in the art, using the Kabat numbering system, the actual linear amino acid sequence of the antibody variable domain can contain fewer or additional amino acids due to a shortening or lengthening of a FR and/or CDR and, as such, an amino acid's Kabat number is not necessarily the same as its linear amino acid number.

As used herein, the term “antigen-binding site” refers to the part of the antigen binding molecule that specifically binds to an antigenic determinant. More particularly, the term “antigen-binding site” refers the part of an antibody that comprises the area which specifically binds to and is complementary to part or all of an antigen. Where an antigen is large, an antigen binding molecule may only bind to a particular part of the antigen, which part is termed an epitope. An antigen-binding site may be provided by, for example, one or more variable domains (also called variable regions). Preferably, an antigen-binding site comprises an antibody light chain variable region (VL) and an antibody heavy chain variable region (VH).

By “specific binding” is meant that the binding is selective for the antigen and can be discriminated from unwanted or non-specific interactions. The ability of an antigen binding molecule to bind to a specific antigen can be measured either through an enzyme-linked immunosorbent assay (ELISA) or other techniques familiar to one of skill in the art, e.g. Surface Plasmon Resonance (SPR) technique (analyzed on a BlAcore instrument) (Liljeblad et al., Glyco J 17, 323-329 (2000)), and traditional binding assays (Heeley, Endocr Res 28, 217-229 (2002)). In one embodiment, the extent of binding of an antigen binding molecule to an unrelated protein is less than about 10% of the binding of the antigen binding molecule to the antigen as measured, e.g. by SPR. In certain embodiments, an molecule that binds to the antigen has a dissociation constant (Kd) of ≤1 μM, ≤100 nM, ≤10 nM, ≤1 nM, ≤0.1 nM, ≤0.01 nM, or ≤0.001 nM (e.g. 10-7 M or less, e.g. from 10-7M to 10-13 M, e.g. from 10-9 M to 10-13 M).

Depending on the amino acid sequence of the constant domain of their heavy chains, immunoglobulins can be assigned to different classes. There are five major classes of human immunoglobulins: IgA, IgD, IgE, IgG, IgM, and IgY, and several of these may be further divided into subclasses (isotypes), e.g., IgG1, IgG2, IgG3, IgG4, IgAl, and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known. Different isotypes have different effector functions. For example, human IgG1 and IgG3 isotypes have ADCC (antibody dependent cell-mediated cytotoxicity) activity. The light chains of antibodies (immunoglobulins) from any vertebrate species can be assigned to one of two clearly distinct types, called kappa (x) and lambda (k), based on the amino acid sequences of their constant domains.

The term “chimeric antibody,” as used herein refers to an antibody in which a portion of the heavy and/or light chain is derived from a particular source (e.g., protein) or species, while the remainder of the heavy and/or light chain is derived from a different source (e.g., protein) or species.

The term “recombinant human antibody,” as used herein, is intended to include all human antibodies that are prepared, expressed, created or isolated by recombinant means, such as antibodies isolated from a host cell such as a NSO or CHO cell or from an animal (e.g. a mouse) that is transgenic for human immunoglobulin genes or antibodies expressed using a recombinant expression vector transfected into a host cell. Such recombinant human antibodies have variable and constant regions in a rearranged form. In some cases, the recombinant human antibodies have been subjected to in vivo somatic hypermutation. Thus, the amino acid sequences of the VH and VL regions of the recombinant antibodies are sequences that, while derived from and related to human germ line VH and VL sequences, may not naturally exist within the human antibody germ line repertoire in vivo.

The term “valent” as used herein denotes the presence of a specified number of binding sites in an antigen binding molecule. As such, the terms “bivalent”, “tetravalent”, and “hexavalent” denote the presence of two binding sites, four binding sites, and six binding sites, respectively, in an antigen binding molecule. The bispecific antibodies according to the invention are at least “bivalent” and may be “trivalent” or “multivalent” (e.g. “tetravalent” or “hexavalent”). In a particular aspect, the antibodies of the present invention have two or more binding sites and are bispecific. That is, the antibodies may be bispecific even in cases where there are more than two binding sites (i.e. that the antibody is trivalent or multivalent). In particular, the invention relates to bispecific bivalent antibodies, having one binding site for each antigen they specifically bind to.

The term “monospecific” antibody as used herein denotes an antibody that has one or more binding sites each of which bind to the same epitope of the same antigen.

The terms “individual(s)”, “subject(s)” and “patient(s)” are used interchangeably herein and refer to any mammal. In some embodiments, the mammal is a human. In some embodiments, the mammal is a non-human. None of the terms require or are limited to situations characterized by the supervision (e.g. constant or intermittent) of a health care worker (e.g. a doctor, a registered nurse, a nurse practitioner, a physician's assistant, an orderly or a hospice worker).

As used herein, the term “percent (%) amino acid sequence identity” with respect to a sequence is defined as the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as EMBOSS MATCHER, EMBOSS WATER, EMBOSS STRETCHER, EMBOSS NEEDLE, EMBOSS LALIGN, BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

The terms “cancer” and “tumor” are used interchangeably herein, encompass all types of oncogenic processes and/or cancerous growths. In embodiments, cancer includes primary tumors as well as metastatic tissues or malignantly transformed cells, tissues, or organs. In embodiments, cancer encompasses all histopathologies and stages, e.g., stages of invasiveness/severity, of a cancer. In embodiments, cancer includes relapsed and/or resistant cancer.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, the molecules of the invention are used to delay development of a disease or to slow the progression of a disease.

### EXAMPLES

These examples are provided for illustrative purposes only and not to limit the scope of the claims provided herein.

### Example 1: Tissue Staining

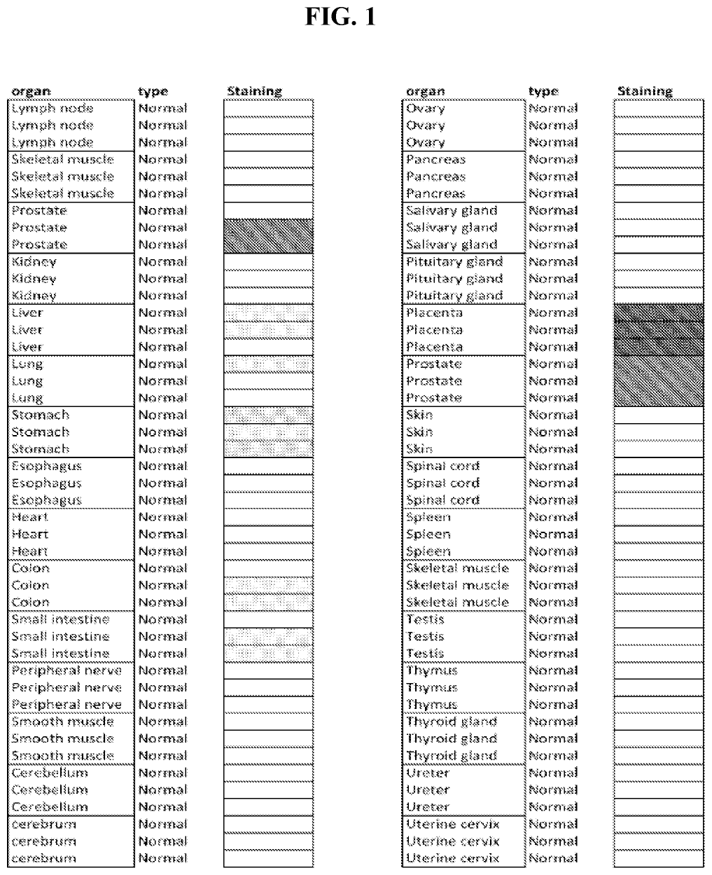

Tissue specificity of the CD46 epitope was determined by immunohistochemistry. Data are shown in FIG. 1. In the figure, shading indicates levels of positive staining with placental trophoblasts being the strongest. Signals in non-shaded ones are either weak or non-detectable.

### Example 2: Preparation of the FOR46 Immunoconjugate

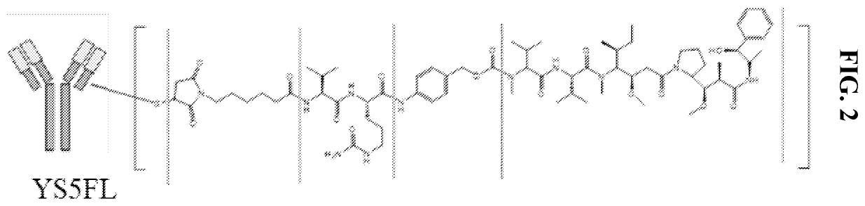

The structure of YS5FL conjugated to an MMAE effector via a mc-vc-PAB linker is shown in FIG. 2. Purified YS5FL mAb (10 mg/ml) is adjusted to a pH of 6.8 with sodium phosphate buffer and then treated with TCEP (TCEP/mAb ratio of 2.1) for two hours at 22° C. Reduced mAb is reacted with mc-vc-PAB-MMAE (drug/mAb ratio of 6) in 9% dimethylacetamide for 15 min. The mAb is reduced a second time for one hour, conjugated a second time for 60 min, and the reaction is quenched by lowering the pH to 5.0 with 1M acetic acid, yielding a FOR46 immunoconjugate with a drug to antibody ratio of about 3.7, as determined by hydrophobic interaction chromatography.

### Example 3: FOR46 Drug Product and Combination with Anti-Cancer Agent

The FOR46 immunoconjugate was formulated into a drug product such that it could be administered to a human subject. The formulation contains 10.0 ±1.0 mg/mL FOR46 drug substance; 20 mM L-histidine buffer, 8.0% (w/v) sucrose, and 0.01% (w/v) polysorbate 80, pH 6.0. The formulation was determined to provide adequate stability (prevention of denaturation of the antibody and prevention of aggregation), buffering, and cryoprotection for storage at -20° C. After storage for 1 month at 5° C., the formulation retained >90% binding potency and cell based activity; was >90% monomeric; had residual MMAE of <15 μg/mL; and was essentially free of visible particles.

The FOR46 drug product thus formulated may be administered in conjunction with a first anti-cancer agent that inreases CD46 expression as described.

### Example 4: Clinical Treatment of Cancer with FOR46 and Anti-Cancer Agent

Clinical trials will be carried out to determine dosing and efficacy of FOR46 in combination with a first anti-cancer agent that increases CD46 expression. The combined drug treatment will be given to human subjects having various cancers as described herein. The greatest improvements are expected in groups of human subjects treated with the combination of FOR46 and the first anti-cancer agent.

Various doses will be assessed. It is expected that a lower dose may be efficacious for FOR46 in combination with the first anti-cancer agent than for FOR46 alone, or for the first anti-cancer agent alone.

### Example 5: Treatment of Cancer with Radiation Followed by FOR46

A patient with metastatic castration resistant prostate cancer was treated with radiation therapy followed by treatment with FOR46. A non-complete response was maintained for at least 102 weeks (34 3-week treatment cycles) as shown in Table 8.

TABLE 8
Sustained FOR46 response after radiation therapy. Serum PSA (mg/ml) levels
and tumor dimensions (RECIST) in a subject with metastatic castration resistant
prostate cancer. Radiation treatment happened between SCR and C1. FOR46 was
administered at 1.2 mg/kg (adjusted body weight) for cycles 1-16. At cycle 17,
the FOR46 dosage was increased to 2.1 mg/kg.

| Cycle | Screen | 1 | 2 | 3 | 4 | 7 | 10 |
|---|---|---|---|---|---|---|---|
| PSA | 78.7 | 9.4 | 2.4 | 0.58 | 0.66 | 1.17 | 4.13 |
| Response | — | — | PSA50 | PSA50 | SD/PSA50 | SD/PSA50 | SD/PSA50 |
| RECIST (mm) | NM | — | — | — | N/N | N/N | N/N |

| Cycle | 14 | 18 | 22 | 26 | 30 | 34 |
|---|---|---|---|---|---|---|
| PSA | 10.4 | 14.2 | 17.4 | 19.3 | 30.5 | 36.7 |
| Response | SD | SD | SD | SD | SD | SD |
| RECIST (mm) | N/N | N/N | N/N | N/N | N/N | N/N |

N/N: Non-complete response, non-progressive disease; SD: stable disease.

### Example 6: Anti-Cancer Agents Increase CD46 Expression

### Methods

Gene expression was determined by RNA-sequencing from formalin-fixed, paraffin-embedded (FFPE) tumor samples using an exome capture-based protocol. Briefly, total nucleic acid was extracted from FFPE solid tumor samples. The RNA sample was treated with DNAse and reverse transcribed. cDNA was captured with a custom probe set targeting the human exome. A library was prepared from the captured cDNA and sequenced on the Illumina platform. Sequence read were aligned to GRCh37 using Kallisto and normalized to quantify gene expression. Specifically, transcript counts were corrected for GC content and length using quantile normalization and adjusted for sequencing depth via a size factor method. Normalized counts in protein coding transcripts covered by the exome panel were then summed to obtain gene-level counts. Subsequent expression analyses were performed on log2-transformed counts. Fernandes et al, Real-world Evidence of Diagnostic Testing and Treatment Patterns in U.S. Breast Cancer Patients with Implications of Treatment Biomarkers fro RNA-sequencing Data, Clin Breast Cancer. 2021 Augusst;21(4): e340-e361.

### Results with Paired Biopsies

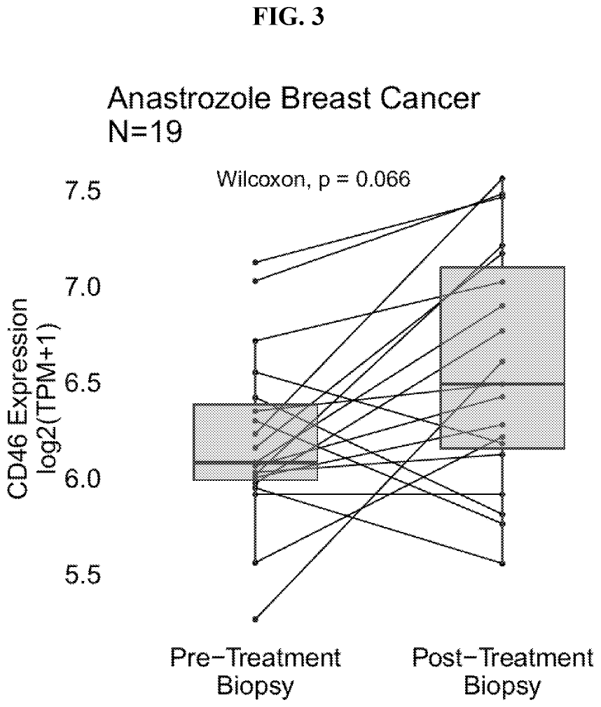

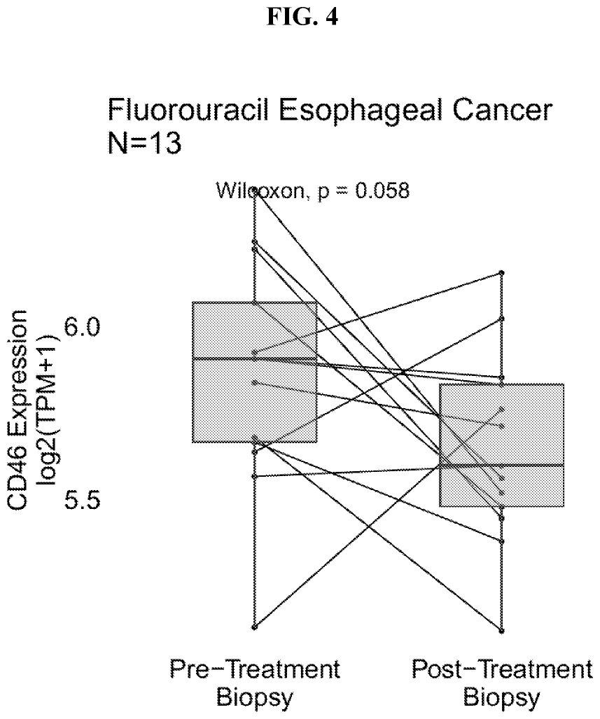

CD46 expression was compared in paired biopsies collected before and after therapy from subjects in 76 therapy settings representing 10 different cancer types as shown in Table 9. CD46 expression increased substantially after treatment of breast cancer with anastrozole as shown in FIG. 3, where CD46 expression increased paired biopsies from 15 of 19 (79%) subjects. In contrast, CD46 expression decreased substantially after treatment of esophageal cancer with fluorouracil as shown in FIG. 4, where CD46 expression decreased in paired biopsies from 10 of 13 (77%) subjects.

### Results with Unpaired Biopsies

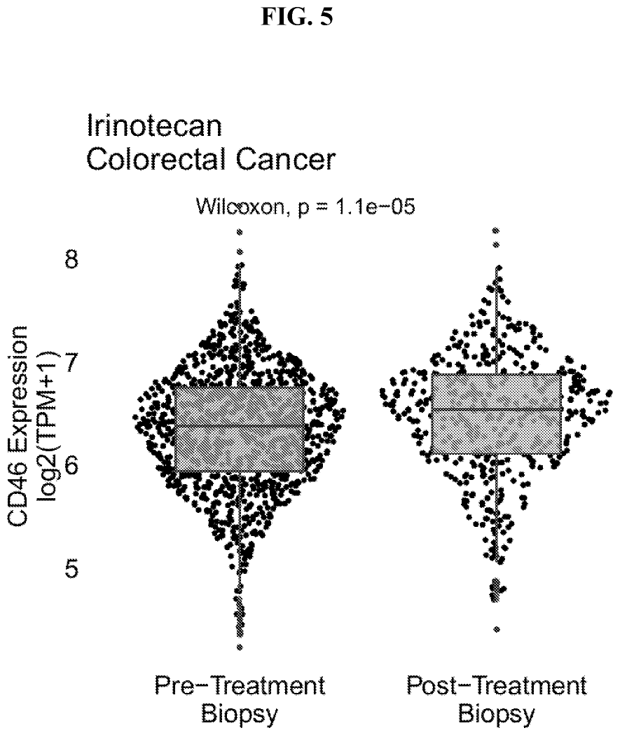

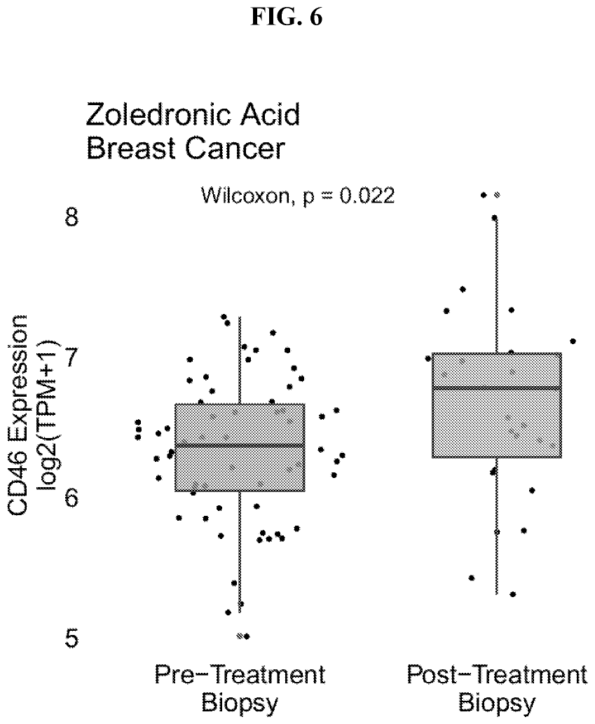

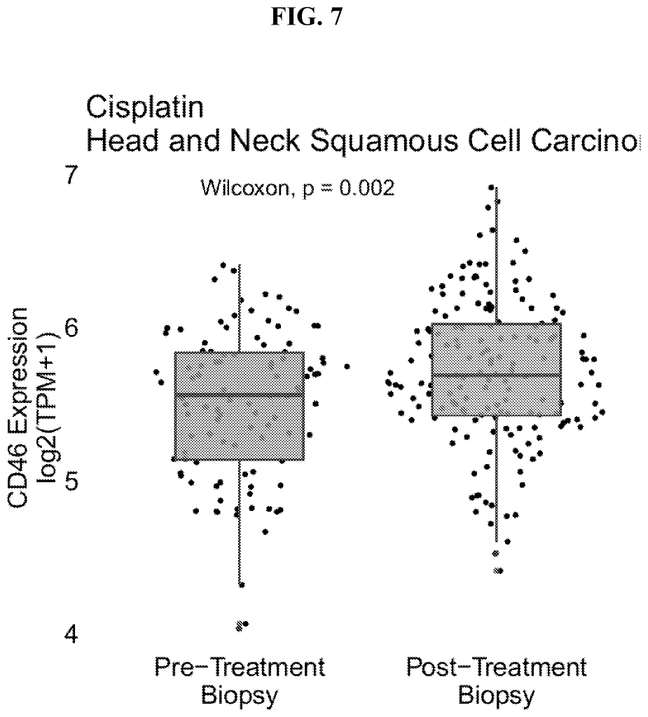

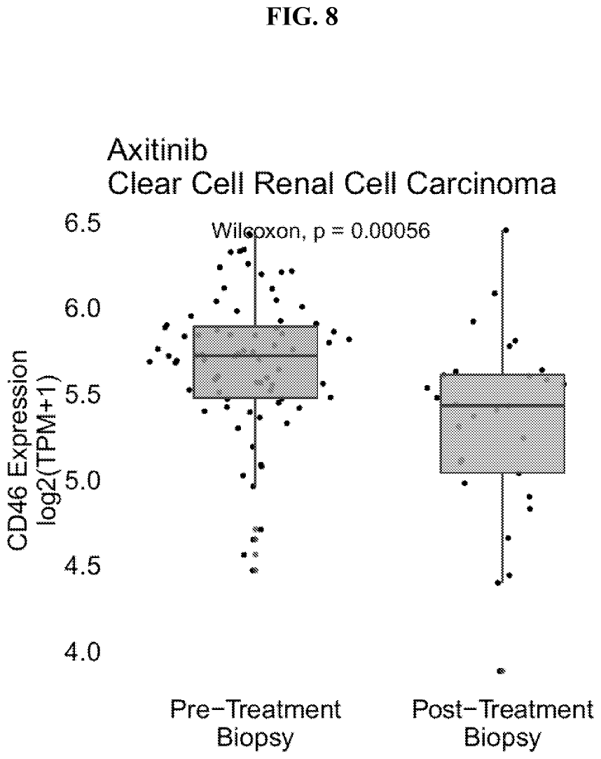

CD46 expression was compared between pre-treatment biopsies collected from subjects known to have received a therapy after the biopsy and post-treatment biopsies collected from subjects that had already been treated when the biopsy was taken as shown in Table 10. CD46 expression was significantly higher after treatment of colorectal cancer with irinotecan as shown in FIG. 5, after treatment of breast cancer with zoledronic acid as shown in FIG. 6, and after treatment of head and neck squamous cell carcinoma with cisplatin as shown in FIG. 7. In contrast, CD46 expression was significantly lower after treatment of clear cell renal cell carcinoma with axitinib as shown in FIG. 8.

TABLE 9
Changes in CD46 expression after cancer therapy-paired biopsies.

| | | | | Pre- | Post- | | |
|---|---|---|---|---|---|---|---|
| | | | | Treatment | Treatment | | Percent |
| Cancer Type | Medication | p-value | N | Biopsy | Biopsy | diff | Change |
| Breast Cancer | anastrozole | 0.065 | 19 | 6.08 | 6.49 | 0.41 | 6.7 |
| Sarcoma | ifosfamide | 0.748 | 11 | 4.76 | 5.08 | 0.32 | 6.7 |
| Ovarian Cancer | gemcitabine | 0.171 | 11 | 5.65 | 5.95 | 0.30 | 5.3 |
| Endometrial | carboplatin | 0.667 | 14 | 5.95 | 6.25 | 0.30 | 5.1 |
| Cancer | | | | | | | |
| Prostate Cancer | abiraterone | 0.563 | 18 | 5.74 | 6.03 | 0.29 | 5.1 |
| Breast Cancer | trastuzumab | 0.246 | 14 | 6.05 | 6.31 | 0.25 | 4.2 |
| Sarcoma | doxorubicin | 0.474 | 17 | 4.99 | 5.19 | 0.20 | 4.1 |
| Colorectal Cancer | regorafenib | 0.151 | 11 | 6.25 | 6.49 | 0.25 | 4.0 |
| Breast Cancer | docetaxel | 0.433 | 17 | 5.97 | 6.19 | 0.22 | 3.7 |
| Endometrial | paclitaxel | 0.769 | 14 | 5.82 | 6.01 | 0.19 | 3.2 |
| Cancer | | | | | | | |
| Pancreatic Cancer | capecitabine | 0.239 | 18 | 5.82 | 5.98 | 0.16 | 2.8 |
| Biliary Cancer | cisplatin | 0.514 | 12 | 5.82 | 5.97 | 0.15 | 2.6 |
| Colorectal Cancer | irinotecan | 0.646 | 64 | 6.33 | 6.48 | 0.16 | 2.5 |
| Prostate Cancer | prednisone | 0.701 | 14 | 5.72 | 5.85 | 0.13 | 2.3 |
| Breast Cancer | doxorubicin | 0.557 | 31 | 6.15 | 6.27 | 0.12 | 1.9 |
| Breast Cancer | tamoxifen | 0.339 | 18 | 6.21 | 6.32 | 0.11 | 1.7 |
| Colorectal Cancer | cetuximab | 0.699 | 11 | 6.03 | 6.13 | 0.10 | 1.7 |
| Bladder Cancer | cisplatin | 0.461 | 15 | 6.15 | 6.25 | 0.10 | 1.6 |
| Pancreatic Cancer | leucovorin | 0.576 | 31 | 5.83 | 5.92 | 0.09 | 1.5 |
| Pancreatic Cancer | fluorouracil | 0.634 | 31 | 5.83 | 5.92 | 0.09 | 1.5 |
| Pancreatic Cancer | gemcitabine | 0.572 | 30 | 5.85 | 5.93 | 0.09 | 1.5 |
| Breast Cancer | gemcitabine | 1.000 | 17 | 6.14 | 6.22 | 0.09 | 1.4 |
| Colorectal Cancer | bevacizumab | 0.698 | 69 | 6.25 | 6.33 | 0.08 | 1.3 |
| Glioblastoma | bevacizumab | 0.684 | 10 | 4.58 | 4.64 | 0.06 | 1.3 |
| Prostate Cancer | cabazitaxel | 0.590 | 12 | 5.86 | 5.93 | 0.07 | 1.2 |
| Prostate Cancer | enzalutamide | 0.448 | 13 | 5.95 | 6.01 | 0.06 | 1.1 |
| Pancreatic Cancer | oxaliplatin | 0.665 | 30 | 5.87 | 5.93 | 0.06 | 1.0 |
| Colorectal Cancer | capecitabine | 0.622 | 50 | 6.25 | 6.31 | 0.06 | 1.0 |
| Biliary Cancer | gemcitabine | 0.734 | 17 | 5.81 | 5.86 | 0.05 | 0.9 |
| Gastric Cancer | oxaliplatin | 0.631 | 10 | 5.64 | 5.69 | 0.05 | 0.8 |
| Glioblastoma | temozolomide | 0.093 | 78 | 4.76 | 4.80 | 0.04 | 0.8 |
| Esophageal Cancer | paclitaxel | 1.000 | 10 | 5.97 | 6.02 | 0.05 | 0.8 |
| Melanoma | pembrolizumab | 0.801 | 13 | 5.22 | 5.25 | 0.03 | 0.6 |
| Sarcoma | gemcitabine | 0.713 | 12 | 5.11 | 5.13 | 0.02 | 0.4 |
| Ovarian Cancer | carboplatin | 0.657 | 58 | 5.66 | 5.67 | 0.01 | 0.2 |
| Breast Cancer | cyclophosphamide | 0.871 | 36 | 6.20 | 6.22 | 0.01 | 0.2 |
| Non-Small Cell | durvalumab | 0.624 | 15 | 5.95 | 5.95 | 0.00 | 0.0 |
| Lung Cancer | | | | | | | |
| Breast Cancer | alpelisib | 0.606 | 11 | 6.58 | 6.57 | −0.01 | −0.1 |
| Pancreatic Cancer | irinotecan | 0.449 | 30 | 5.94 | 5.93 | −0.01 | −0.1 |
| Pancreatic Cancer | paclitaxel protein- | 0.795 | 19 | 5.97 | 5.96 | −0.01 | −0.2 |
| | bound | | | | | | |
| Prostate Cancer | docetaxel | 0.322 | 17 | 5.78 | 5.76 | −0.03 | −0.4 |
| Breast Cancer | exemestane | 1.000 | 14 | 6.41 | 6.37 | −0.04 | 0.5 |
| Breast Cancer | paclitaxel protein- | 0.921 | 21 | 6.18 | 6.14 | −0.03 | −0.6 |
| | bound | | | | | | |
| Breast Cancer | letrozole | 0.921 | 26 | 6.45 | 6.41 | −0.04 | −0.6 |
| Colorectal Cancer | oxaliplatin | 0.520 | 98 | 6.30 | 6.26 | −0.04 | −0.6 |
| Melanoma | nivolumab | 0.792 | 26 | 5.29 | 5.26 | −0.04 | −0.7 |
| Ovarian Cancer | paclitaxel | 0.415 | 53 | 5.71 | 5.67 | −0.04 | −0.8 |
| Colorectal Cancer | fluorouracil | 0.627 | 109 | 6.32 | 6.27 | −0.05 | −0.8 |
| Bladder Cancer | gemcitabine | 0.454 | 14 | 6.29 | 6.24 | −0.05 | −0.8 |
| Breast Cancer | capecitabine | 0.890 | 41 | 6.29 | 6.23 | −0.06 | −0.9 |
| Non-Small Cell | carboplatin | 0.763 | 60 | 6.13 | 6.07 | −0.06 | −1.0 |
| Lung Cancer | | | | | | | |
| Colorectal Cancer | leucovorin | 0.838 | 104 | 6.36 | 6.30 | −0.07 | −1.0 |
| Melanoma | ipilimumab | 0.673 | 18 | 5.31 | 5.26 | −0.06 | −1.1 |
| Breast Cancer | carboplatin | 0.877 | 27 | 6.14 | 6.06 | −0.08 | −1.2 |
| Breast Cancer | paclitaxel | 0.636 | 32 | 6.21 | 6.12 | −0.09 | −1.4 |
| Colorectal Cancer | panitumumab | 0.631 | 10 | 6.71 | 6.62 | −0.10 | −1.4 |
| Gastric Cancer | fluorouracil | 0.362 | 13 | 5.74 | 5.66 | −0.09 | −1.5 |
| Breast Cancer | pembrolizumab | 0.734 | 14 | 6.28 | 6.17 | −0.11 | −1.7 |
| Ovarian Cancer | bevacizumab | 0.367 | 15 | 5.65 | 5.54 | −0.11 | −1.9 |
| Breast Cancer | fulvestrant | 0.885 | 45 | 6.58 | 6.44 | −0.13 | −2.1 |
| Ovarian Cancer | doxorubicin | 0.148 | 15 | 5.66 | 5.54 | −0.13 | −2.2 |
| | liposome | | | | | | |
| Esophageal Cancer | leucovorin | 0.246 | 14 | 5.87 | 5.74 | −0.14 | −2.3 |
| Breast Cancer | atezolizumab | 0.888 | 18 | 6.14 | 5.99 | −0.14 | −2.4 |
| Ovarian Cancer | olaparib | 0.218 | 10 | 5.65 | 5.51 | −0.14 | −2.5 |
| Breast Cancer | palbociclib | 0.372 | 33 | 6.51 | 6.34 | −0.17 | −2.7 |
| Esophageal Cancer | oxaliplatin | 0.164 | 14 | 5.91 | 5.74 | −0.17 | −2.9 |
| Colorectal Cancer | dexamethasone | 0.898 | 11 | 6.15 | 5.96 | −0.19 | −3.0 |
| Prostate Cancer | leuprolide | 0.081 | 25 | 6.11 | 5.92 | −0.19 | −3.1 |
| Non-Small Cell | pemetrexed | 0.157 | 43 | 6.42 | 6.22 | −0.20 | −3.1 |
| Lung Cancer | | | | | | | |
| Non-Small Cell | paclitaxel | 0.631 | 25 | 6.12 | 5.89 | −0.23 | −3.7 |
| Lung Cancer | | | | | | | |
| Prostate Cancer | bicalutamide | 0.169 | 13 | 6.32 | 6.08 | −0.24 | −3.8 |
| Non-Small Cell | pembrolizumab | 0.450 | 31 | 6.23 | 6.00 | −0.24 | −3.8 |
| Lung Cancer | | | | | | | |
| Non-Small Cell | osimertinib | 0.211 | 22 | 6.50 | 6.25 | −0.26 | −4.0 |
| Lung Cancer | | | | | | | |
| Esophageal Cancer | fluorouracil | 0.057 | 13 | 5.91 | 5.60 | −0.31 | −5.2 |
| Non-Small Cell | cisplatin | 0.285 | 15 | 6.31 | 5.85 | −0.45 | −7.2 |
| Lung Cancer | | | | | | | |
| Breast Cancer | eribulin | 0.266 | 12 | 6.47 | 5.92 | −0.54 | −8.4 |

TABLE 10
Changes in CD46 expression after cancer therapy-unpaired biopsies.

| | | | | | | | | | Percentage |
|---|---|---|---|---|---|---|---|---|---|
| Cancer type | Medication | p-value | n pre | n post | FDR | Pre | Post | diff | change |
| Colorectal | carboplatin | 0.0065 | 30 | 20 | 0.0647 | 5.76 | 6.35 | 0.59 | 10.3 |
| Cancer | | | | | | | | | |
| Breast | zoledronic acid | 0.0221 | 62 | 27 | 0.0221 | 6.37 | 6.78 | 0.41 | 6.4 |
| Cancer | | | | | | | | | |
| Breast | denosumab | 0.0016 | 58 | 22 | 0.0016 | 6.42 | 6.82 | 0.40 | 6.2 |
| Cancer | | | | | | | | | |
| Sarcoma | etoposide | 0.0171 | 34 | 65 | 0.0514 | 4.83 | 5.13 | 0.30 | 6.2 |
| Breast | bevacizumab | 0.5530 | 25 | 14 | 0.8030 | 6.01 | 6.34 | 0.33 | 5.4 |
| Cancer | | | | | | | | | |
| Breast | methylprednisolone | 0.4457 | 33 | 24 | 0.8914 | 6.18 | 6.49 | 0.31 | 5.0 |
| Cancer | | | | | | | | | |
| Colorectal | cetuximab | 0.0020 | 173 | 96 | 0.0040 | 6.22 | 6.53 | 0.31 | 5.0 |
| Cancer | | | | | | | | | |
| Colorectal | pembrolizumab | 0.5340 | 81 | 24 | 0.7476 | 6.20 | 6.50 | 0.30 | 4.8 |
| Cancer | | | | | | | | | |
| Endometrial | cisplatin | 0.1686 | 50 | 25 | 0.3793 | 5.73 | 5.99 | 0.26 | 4.6 |
| Cancer | | | | | | | | | |
| Colorectal | pegfilgrastim | 0.1008 | 79 | 17 | 0.3023 | 6.33 | 6.62 | 0.29 | 4.6 |
| Cancer | | | | | | | | | |
| Colorectal | panitumumab | 0.0008 | 169 | 81 | 0.0008 | 6.36 | 6.64 | 0.28 | 4.4 |
| Cancer | | | | | | | | | |
| Sarcoma | doxorubicin | 0.5007 | 42 | 28 | 0.6676 | 4.96 | 5.17 | 0.22 | 4.3 |
| | liposome | | | | | | | | |
| Colorectal | metformin | 0.2693 | 28 | 17 | 0.5386 | 6.43 | 6.70 | 0.27 | 4.2 |
| Cancer | | | | | | | | | |
| Head and | cetuximab | 0.0040 | 64 | 44 | 0.0040 | 5.55 | 5.78 | 0.23 | 4.2 |
| Neck | | | | | | | | | |
| Squamous | | | | | | | | | |
| Cell | | | | | | | | | |
| Carcinoma | | | | | | | | | |
| Esophageal | trastuzumab | 0.3039 | 66 | 33 | 0.6079 | 5.89 | 6.13 | 0.24 | 4.0 |
| Cancer | | | | | | | | | |
| Colorectal | mitomycin | 0.7583 | 25 | 25 | 0.7583 | 5.71 | 5.93 | 0.23 | 3.9 |
| Cancer | | | | | | | | | |
| Sarcoma | cisplatin | 0.1156 | 33 | 63 | 0.3467 | 4.83 | 5.02 | 0.19 | 3.9 |
| Endocrine | carboplatin | 0.1118 | 68 | 25 | 0.2795 | 5.68 | 5.90 | 0.22 | 3.9 |
| Tumor | | | | | | | | | |
| Breast | endocrine therapy | 0.1736 | 48 | 17 | 0.3472 | 6.08 | 6.29 | 0.21 | 3.5 |
| Cancer | | | | | | | | | |
| Sarcoma | ifosfamide | 0.0940 | 124 | 200 | 0.0940 | 4.90 | 5.07 | 0.17 | 3.4 |
| Colorectal | aflibercept | 0.2902 | 36 | 26 | 0.2902 | 6.37 | 6.58 | 0.21 | 3.3 |
| Cancer | | | | | | | | | |
| Head and | pembrolizumab | 0.2616 | 116 | 43 | 0.6104 | 5.59 | 5.76 | 0.17 | 3.1 |
| Neck | | | | | | | | | |
| Squamous | | | | | | | | | |
| Cell | | | | | | | | | |
| Carcinoma | | | | | | | | | |
| Pancreatic | cisplatin | 0.5071 | 23 | 18 | 0.7606 | 5.98 | 6.16 | 0.18 | 3.1 |
| Cancer | | | | | | | | | |
| Colorectal | regorafenib | 0.0782 | 137 | 43 | 0.0782 | 6.44 | 6.63 | 0.19 | 2.9 |
| Cancer | | | | | | | | | |
| Non-Small | crizotinib | 0.3782 | 21 | 33 | 0.3782 | 5.99 | 6.16 | 0.16 | 2.7 |
| Cell Lung | | | | | | | | | |
| Cancer | | | | | | | | | |
| Colorectal | irinotecan | 1.10E−05 | 1014 | 479 | 3.3E−05 | 6.38 | 6.54 | 0.16 | 2.5 |
| Cancer | | | | | | | | | |
| Head and | cisplatin | 0.0020 | 103 | 166 | 0.0182 | 5.56 | 5.69 | 0.13 | 2.4 |
| Neck | | | | | | | | | |
| Squamous | | | | | | | | | |
| Cell | | | | | | | | | |
| Carcinoma | | | | | | | | | |
| Head and | paclitaxel | 0.2558 | 75 | 50 | 0.6821 | 5.55 | 5.68 | 0.13 | 2.4 |
| Neck | | | | | | | | | |
| Squamous | | | | | | | | | |
| Cell | | | | | | | | | |
| Carcinoma | | | | | | | | | |
| Sarcoma | olaratumab | 0.1824 | 40 | 36 | 0.1824 | 4.99 | 5.10 | 0.11 | 2.3 |
| Bladder | carboplatin | 0.2609 | 144 | 55 | 0.4348 | 5.99 | 6.12 | 0.14 | 2.3 |
| Cancer | | | | | | | | | |
| Ovarian | docetaxel | 0.1889 | 81 | 54 | 0.2834 | 5.76 | 5.88 | 0.12 | 2.1 |
| Cancer | | | | | | | | | |
| Breast | alpelisib | 0.3754 | 91 | 28 | 0.3754 | 6.53 | 6.67 | 0.13 | 2.0 |
| Cancer | | | | | | | | | |
| Head and | carboplatin | 0.0660 | 109 | 65 | 0.2199 | 5.58 | 5.69 | 0.11 | 2.0 |
| Neck | | | | | | | | | |
| Squamous | | | | | | | | | |
| Cell | | | | | | | | | |
| Carcinoma | | | | | | | | | |
| Endocrine | etoposide | 0.1333 | 83 | 29 | 0.1333 | 5.67 | 5.79 | 0.11 | 2.0 |
| Tumor | | | | | | | | | |
| Melanoma | dabrafenib | 0.0614 | 51 | 34 | 0.0614 | 5.31 | 5.42 | 0.11 | 2.0 |
| Ovarian | cyclophosphamide | 0.7446 | 39 | 26 | 0.9900 | 5.66 | 5.76 | 0.11 | 1.9 |
| Cancer | | | | | | | | | |
| Colorectal | antineoplastic | 0.6644 | 37 | 49 | 0.6644 | 6.28 | 6.39 | 0.12 | 1.8 |
| Cancer | agents | | | | | | | | |
| Colorectal | tipiracil/trifluridine | 0.0888 | 134 | 43 | 0.0888 | 6.50 | 6.62 | 0.12 | 1.8 |
| Cancer | | | | | | | | | |
| Breast | tamoxifen | 0.0439 | 170 | 493 | 0.0439 | 6.27 | 6.38 | 0.11 | 1.8 |
| Cancer | | | | | | | | | |
| Breast | eribulin | 0.2274 | 178 | 128 | 0.2274 | 6.09 | 6.20 | 0.11 | 1.8 |
| Cancer | | | | | | | | | |
| Breast | exemestane | 0.1067 | 184 | 275 | 0.1067 | 6.38 | 6.50 | 0.11 | 1.8 |
| Cancer | | | | | | | | | |
| Biliary | capecitabine | 0.4580 | 85 | 58 | 0.7705 | 5.77 | 5.87 | 0.10 | 1.8 |
| Cancer | | | | | | | | | |
| Non-Small | gemcitabine | 0.5772 | 193 | 83 | 0.8081 | 5.88 | 5.98 | 0.10 | 1.7 |
| Cell Lung | | | | | | | | | |
| Cancer | | | | | | | | | |
| Colorectal | bevacizumab | 0.0164 | 1133 | 494 | 0.0982 | 6.42 | 6.53 | 0.11 | 1.7 |
| Cancer | | | | | | | | | |
| Non-Small | afatinib | 0.6507 | 31 | 37 | 0.6507 | 6.32 | 6.43 | 0.11 | 1.7 |
| Cell Lung | | | | | | | | | |
| Cancer | | | | | | | | | |
| Non-Small | etoposide | 0.0838 | 127 | 120 | 0.1256 | 5.88 | 5.98 | 0.10 | 1.7 |
| Cell Lung | | | | | | | | | |
| Cancer | | | | | | | | | |
| Glioblastoma | temozolomide | 0.0031 | 852 | 319 | 0.0062 | 4.78 | 4.85 | 0.08 | 1.6 |
| Prostate | cabazitaxel | 0.1917 | 137 | 80 | 0.1917 | 6.04 | 6.13 | 0.09 | 1.6 |
| Cancer | | | | | | | | | |
| Esophageal | pembrolizumab | 0.1330 | 55 | 26 | 0.6104 | 5.93 | 6.01 | 0.09 | 1.4 |
| Cancer | | | | | | | | | |
| Melanoma | trametinib | 0.1460 | 51 | 37 | 0.1460 | 5.32 | 5.39 | 0.08 | 1.4 |
| Breast | everolimus | 0.5240 | 120 | 119 | 0.5240 | 6.38 | 6.46 | 0.09 | 1.4 |
| Cancer | | | | | | | | | |
| Sarcoma | doxorubicin | 0.2380 | 242 | 281 | 0.3570 | 4.99 | 5.06 | 0.07 | 1.3 |
| Breast | atezolizumab | 0.7051 | 135 | 68 | 0.8826 | 5.92 | 6.00 | 0.07 | 1.3 |
| Cancer | | | | | | | | | |
| Colorectal | leucovorin | 0.0021 | 1829 | 1011 | 0.0053 | 6.38 | 6.46 | 0.08 | 1.2 |
| Cancer | | | | | | | | | |
| Non-Small | pegfilgrastim | 0.5975 | 132 | 24 | 0.6449 | 5.99 | 6.06 | 0.07 | 1.2 |
| Cell Lung | | | | | | | | | |
| Cancer | | | | | | | | | |
| Pancreatic | capecitabine | 0.0335 | 232 | 175 | 0.2347 | 5.86 | 5.93 | 0.07 | 1.2 |
| Cancer | | | | | | | | | |
| Breast | methotrexate | 0.3369 | 37 | 33 | 0.3369 | 6.25 | 6.32 | 0.07 | 1.2 |
| Cancer | | | | | | | | | |
| Sarcoma | cyclophosphamide | 0.7863 | 48 | 70 | 0.9900 | 5.06 | 5.12 | 0.06 | 1.2 |
| Endocrine | capecitabine | 0.6355 | 15 | 8 | 0.7705 | 5.60 | 5.66 | 0.06 | 1.1 |
| Tumor | | | | | | | | | |
| Breast | paclitaxel | 0.5005 | 274 | 177 | 0.8342 | 6.07 | 6.14 | 0.06 | 1.0 |
| Cancer | protein-bound | | | | | | | | |
| Breast | fluorouracil | 0.1246 | 46 | 57 | 0.2181 | 6.12 | 6.18 | 0.06 | 1.0 |
| Cancer | | | | | | | | | |
| Colorectal | dexamethasone | 0.1925 | 276 | 61 | 0.3208 | 6.38 | 6.44 | 0.06 | 1.0 |
| Cancer | | | | | | | | | |
| Ovarian | doxorubicin | 0.1530 | 383 | 120 | 0.4662 | 5.69 | 5.75 | 0.06 | 1.0 |
| Cancer | liposome | | | | | | | | |
| Colorectal | fluorouracil | 0.0312 | 1914 | 1059 | 0.0728 | 6.38 | 6.44 | 0.06 | 0.9 |
| Cancer | | | | | | | | | |
| Bladder | pembrolizumab | 0.9168 | 198 | 63 | 0.9168 | 6.12 | 6.18 | 0.06 | 0.9 |
| Cancer | | | | | | | | | |
| Biliary | oxaliplatin | 0.3845 | 68 | 34 | 0.4807 | 5.85 | 5.91 | 0.05 | 0.9 |
| Cancer | | | | | | | | | |
| Breast | anastrozole | 0.5268 | 263 | 598 | 0.5268 | 6.35 | 6.41 | 0.06 | 0.9 |
| Cancer | | | | | | | | | |
| Esophageal | capecitabine | 0.7243 | 40 | 28 | 0.7705 | 5.98 | 6.02 | 0.05 | 0.8 |
| Cancer | | | | | | | | | |
| Colorectal | capecitabine | 0.1277 | 732 | 537 | 0.4468 | 6.39 | 6.43 | 0.05 | 0.8 |
| Cancer | | | | | | | | | |
| Breast | docetaxel | 0.5435 | 209 | 537 | 0.6521 | 6.19 | 6.23 | 0.04 | 0.7 |
| Cancer | | | | | | | | | |
| Gastric | paclitaxel | 0.9715 | 41 | 22 | 0.9715 | 5.78 | 5.82 | 0.04 | 0.7 |
| Cancer | | | | | | | | | |
| Breast | olaparib | 0.9037 | 58 | 26 | 0.9037 | 6.12 | 6.15 | 0.04 | 0.6 |
| Cancer | | | | | | | | | |
| Colorectal | oxaliplatin | 0.0509 | 1805 | 1109 | 0.0848 | 6.38 | 6.42 | 0.04 | 0.6 |
| Cancer | | | | | | | | | |
| Ovarian | cisplatin | 0.2674 | 120 | 61 | 0.4814 | 5.76 | 5.79 | 0.03 | 0.6 |
| Cancer | | | | | | | | | |
| Endometrial | paclitaxel | 0.8242 | 574 | 241 | 0.9715 | 5.82 | 5.85 | 0.03 | 0.6 |
| Cancer | | | | | | | | | |
| Head and | fluorouracil | 0.9009 | 57 | 27 | 0.9009 | 5.59 | 5.63 | 0.03 | 0.6 |
| Neck | | | | | | | | | |
| Squamous | | | | | | | | | |
| Cell | | | | | | | | | |
| Carcinoma | | | | | | | | | |
| Pancreatic | irinotecan liposomal | 0.4182 | 59 | 27 | 0.4182 | 5.92 | 5.95 | 0.03 | 0.6 |
| Cancer | | | | | | | | | |
| Ovarian | bevacizumab | 0.7248 | 522 | 212 | 0.8030 | 5.72 | 5.75 | 0.03 | 0.5 |
| Cancer | | | | | | | | | |
| Non-Small | metformin | 0.9704 | 53 | 31 | 0.9704 | 6.04 | 6.07 | 0.03 | 0.5 |
| Cell Lung | | | | | | | | | |
| Cancer | | | | | | | | | |
| Prostate | enzalutamide | 0.7274 | 415 | 278 | 0.7274 | 6.11 | 6.14 | 0.03 | 0.4 |
| Cancer | | | | | | | | | |
| Non-Small | paclitaxel | 0.6879 | 114 | 40 | 0.8598 | 5.87 | 5.90 | 0.03 | 0.4 |
| Cell Lung | protein-bound | | | | | | | | |
| Cancer | | | | | | | | | |
| Breast | gemcitabine | 0.2010 | 222 | 16 | 0.4691 | 6.07 | 6.09 | 0.02 | 0.3 |
| Cancer | | | | | | | | | |
| Breast | filgrastim | 0.8657 | 35 | 27 | 0.8657 | 6.21 | 6.23 | 0.02 | 0.3 |
| Cancer | | | | | | | | | |
| Non-Small | docetaxel | 0.9749 | 215 | 80 | 0.9749 | 6.11 | 6.13 | 0.02 | 0.3 |
| Cell Lung | | | | | | | | | |
| Cancer | | | | | | | | | |
| Non-Small | prednisone | 0.8008 | 152 | 60 | 0.8008 | 6.11 | 6.13 | 0.02 | 0.3 |
| Cell Lung | | | | | | | | | |
| Cancer | | | | | | | | | |
| Breast | cisplatin | 0.8607 | 37 | 27 | 0.9146 | 5.97 | 5.98 | 0.02 | 0.3 |
| Cancer | | | | | | | | | |
| Endometrial | carboplatin | 0.9267 | 604 | 260 | 0.9806 | 5.82 | 5.83 | 0.01 | 0.2 |
| Cancer | | | | | | | | | |
| Breast | palbociclib | 0.9504 | 598 | 464 | 0.9504 | 6.41 | 6.42 | 0.01 | 0.2 |
| Cancer | | | | | | | | | |
| Pancreatic | gemcitabine | 0.1623 | 705 | 382 | 0.4691 | 5.87 | 5.87 | 0.00 | 0.1 |
| Cancer | | | | | | | | | |
| Biliary | leucovorin | 0.4752 | 52 | 35 | 0.5939 | 5.92 | 5.92 | 0.00 | 0.1 |
| Cancer | | | | | | | | | |
| Ovarian | niraparib | 0.9329 | 170 | 58 | 0.9329 | 5.72 | 5.72 | 0.00 | 0.1 |
| Cancer | | | | | | | | | |
| Melanoma | pembrolizumab | 0.6459 | 150 | 126 | 0.7535 | 5.30 | 5.30 | 0.00 | 0.0 |
| Non-Small | dexamethasone | 0.6706 | 453 | 126 | 0.6706 | 6.13 | 6.14 | 0.00 | 0.0 |
| Cell Lung | | | | | | | | | |
| Cancer | | | | | | | | | |
| Biliary | fluorouracil | 0.3904 | 61 | 46 | 0.5465 | 5.92 | 5.92 | 0.00 | 0.0 |
| Cancer | | | | | | | | | |
| Sarcoma | trabectedin | 0.5627 | 38 | 33 | 0.5627 | 5.24 | 5.23 | 0.00 | −0.1 |
| Breast | goserelin | 0.7953 | 64 | 96 | 0.7953 | 6.39 | 6.38 | 0.00 | −0.1 |
| Cancer | | | | | | | | | |
| Non-Small | atezolizumab | 0.8826 | 81 | 45 | 0.8826 | 6.05 | 6.04 | 0.00 | −0.1 |
| Cell Lung | | | | | | | | | |
| Cancer | | | | | | | | | |
| Ovarian | paclitaxel | 0.9327 | 1130 | 741 | 0.9715 | 5.73 | 5.73 | −0.01 | −0.1 |
| Cancer | | | | | | | | | |
| Ovarian | carboplatin | 0.9806 | 1248 | 764 | 0.9806 | 5.74 | 5.73 | −0.01 | −0.2 |
| Cancer | | | | | | | | | |
| Ovarian | olaparib | 0.6083 | 202 | 65 | 0.9037 | 5.72 | 5.70 | −0.02 | −0.3 |
| Cancer | | | | | | | | | |
| Breast | doxorubicin | 0.9537 | 326 | 991 | 0.9537 | 6.06 | 6.05 | −0.02 | −0.3 |
| Cancer | | | | | | | | | |
| Thyroid | lenvatinib | 0.5422 | 36 | 23 | 0.5422 | 5.84 | 5.82 | −0.02 | −0.4 |
| Cancer | | | | | | | | | |
| Prostate | sipuleucel-t | 0.8189 | 59 | 72 | 0.8189 | 6.10 | 6.08 | −0.02 | −0.4 |
| Cancer | | | | | | | | | |
| Glioblastoma | bevacizumab | 0.8030 | 251 | 37 | 0.8030 | 4.81 | 4.79 | −0.02 | −0.4 |
| Breast | cyclophosphamide | 0.9900 | 420 | 1190 | 0.9900 | 6.11 | 6.08 | −0.03 | −0.4 |
| Cancer | | | | | | | | | |
| Prostate | apalutamide | 0.3103 | 81 | 27 | 0.3103 | 6.17 | 6.14 | −0.03 | −0.5 |
| Cancer | | | | | | | | | |
| Melanoma | nivolumab | 0.7977 | 283 | 226 | 0.9581 | 5.33 | 5.31 | −0.02 | −0.5 |
| Prostate | carboplatin | 0.7388 | 93 | 36 | 0.9235 | 5.95 | 5.92 | −0.03 | −0.5 |
| Cancer | | | | | | | | | |
| Sarcoma | vincristine | 0.9930 | 52 | 60 | 0.9930 | 5.12 | 5.09 | −0.03 | −0.5 |
| Biliary | gemcitabine | 0.9264 | 156 | 95 | 0.9648 | 5.83 | 5.80 | −0.03 | −0.5 |
| Cancer | | | | | | | | | |
| Non-Small | antineoplastic | 0.5056 | 28 | 36 | 0.6644 | 6.04 | 6.01 | −0.03 | −0.5 |
| Cell Lung | agents | | | | | | | | |
| Cancer | | | | | | | | | |
| Non-Small | durvalumab | 0.8143 | 263 | 224 | 0.8143 | 6.07 | 6.04 | −0.03 | −0.5 |
| Cell Lung | | | | | | | | | |
| Cancer | | | | | | | | | |
| Bladder | gemcitabine | 0.2895 | 280 | 220 | 0.5067 | 6.15 | 6.12 | −0.03 | −0.5 |
| Cancer | | | | | | | | | |
| Non-Small | alectinib | 0.9226 | 34 | 37 | 0.9226 | 6.22 | 6.18 | −0.03 | −0.5 |
| Cell Lung | | | | | | | | | |
| Cancer | | | | | | | | | |
| Breast | trastuzumab | 0.7062 | 173 | 305 | 0.7062 | 6.29 | 6.25 | −0.03 | −0.6 |
| Cancer | | | | | | | | | |
| Breast | letrozole | 0.7194 | 554 | 592 | 0.7797 | 6.42 | 6.39 | −0.04 | −0.6 |
| Cancer | | | | | | | | | |
| Non-Small | erlotinib | 0.5287 | 52 | 95 | 0.5287 | 6.27 | 6.23 | −0.04 | −0.6 |
| Cell Lung | | | | | | | | | |
| Cancer | | | | | | | | | |
| Non-Small | methylprednisolone | 0.9261 | 77 | 34 | 0.9261 | 6.20 | 6.16 | −0.04 | −0.6 |
| Cell Lung | | | | | | | | | |
| Cancer | | | | | | | | | |
| Gastric | capecitabine | 0.7705 | 43 | 33 | 0.7705 | 5.77 | 5.73 | −0.04 | −0.7 |
| Cancer | | | | | | | | | |
| Non-Small | pembrolizumab | 0.4564 | 1033 | 318 | 0.7476 | 6.21 | 6.16 | −0.05 | −0.7 |
| Cell Lung | | | | | | | | | |
| Cancer | | | | | | | | | |
| Endocrine | temozolomide | 0.5366 | 18 | 6 | 0.5366 | 5.56 | 5.52 | −0.04 | −0.8 |
| Tumor | | | | | | | | | |
| Non-Small | cisplatin | 0.6574 | 335 | 245 | 0.8453 | 6.16 | 6.11 | −0.05 | −0.8 |
| Cell Lung | | | | | | | | | |
| Cancer | | | | | | | | | |
| Pancreatic | paclitaxel protein- | 0.4317 | 477 | 294 | 0.8342 | 5.90 | 5.85 | −0.05 | −0.8 |
| Cancer | bound | | | | | | | | |
| Breast | ado-trastuzumab | 0.4383 | 74 | 117 | 0.4383 | 6.25 | 6.20 | −0.05 | −0.8 |
| Cancer | emtansine | | | | | | | | |
| Ovarian | gemcitabine | 0.9648 | 252 | 106 | 0.9648 | 5.77 | 5.72 | −0.05 | −0.9 |
| Cancer | | | | | | | | | |
| Esophageal | leucovorin | 0.8104 | 272 | 90 | 0.8104 | 5.89 | 5.84 | −0.05 | −0.9 |
| Cancer | | | | | | | | | |
| Endocrine | octreotide | 0.3331 | 19 | 16 | 0.3331 | 5.64 | 5.59 | −0.05 | −0.9 |
| Tumor | | | | | | | | | |
| Prostate | bicalutamide | 0.0460 | 524 | 363 | 0.0460 | 6.18 | 6.13 | −0.06 | −0.9 |
| Cancer | | | | | | | | | |
| Prostate | endocrine therapy | 0.4718 | 102 | 90 | 0.4718 | 6.17 | 6.11 | −0.06 | −1.0 |
| Cancer | | | | | | | | | |
| Esophageal | oxaliplatin | 0.7516 | 281 | 103 | 0.7516 | 5.90 | 5.85 | −0.06 | −1.0 |
| Cancer | | | | | | | | | |
| Esophageal | fluorouracil | 0.5074 | 288 | 107 | 0.5920 | 5.89 | 5.83 | −0.06 | −1.0 |
| Cancer | | | | | | | | | |
| Endometrial | doxorubicin | 0.9179 | 85 | 21 | 0.9179 | 5.79 | 5.73 | −0.06 | −1.0 |
| Cancer | liposome | | | | | | | | |
| Biliary | cisplatin | 0.9146 | 83 | 79 | 0.9146 | 5.82 | 5.76 | −0.06 | −1.0 |
| Cancer | | | | | | | | | |
| Breast | dexamethasone | 0.5931 | 123 | 75 | 0.6706 | 6.27 | 6.20 | −0.07 | −1.1 |
| Cancer | | | | | | | | | |
| Melanoma | ipilimumab | 0.2458 | 186 | 145 | 0.4916 | 5.36 | 5.30 | −0.06 | −1.1 |
| Non-Small | paclitaxel | 0.6542 | 662 | 388 | 0.9715 | 6.06 | 6.00 | −0.07 | −1.1 |
| Cell Lung | | | | | | | | | |
| Cancer | | | | | | | | | |
| Breast | carboplatin | 0.6506 | 329 | 463 | 0.9235 | 6.07 | 6.00 | −0.07 | −1.1 |
| Cancer | | | | | | | | | |
| Clear Cell | nivolumab | 0.2653 | 151 | 56 | 0.7958 | 5.56 | 5.50 | −0.06 | −1.1 |
| Renal Cell | | | | | | | | | |
| Carcinoma | | | | | | | | | |
| Prostate | abiraterone | 0.0274 | 585 | 295 | 0.0274 | 6.17 | 6.10 | −0.07 | −1.1 |
| Cancer | | | | | | | | | |
| Breast | capecitabine | 0.1921 | 498 | 467 | 0.4481 | 6.22 | 6.15 | −0.07 | −1.2 |
| Cancer | | | | | | | | | |
| Breast | fulvestrant | 0.2446 | 531 | 413 | 0.2446 | 6.48 | 6.41 | −0.08 | −1.2 |
| Cancer | | | | | | | | | |
| Breast | lapatinib | 0.1756 | 31 | 28 | 0.1756 | 6.30 | 6.22 | −0.08 | −1.2 |
| Cancer | | | | | | | | | |
| Non-Small | carboplatin | 0.1936 | 1585 | 787 | 0.3871 | 6.18 | 6.10 | −0.08 | −1.2 |
| Cell Lung | | | | | | | | | |
| Cancer | | | | | | | | | |
| Breast | pertuzumab | 0.8009 | 126 | 246 | 0.8009 | 6.33 | 6.25 | −0.08 | −1.3 |
| Cancer | | | | | | | | | |
| Clear Cell | ipilimumab | 0.6258 | 106 | 33 | 0.6258 | 5.55 | 5.48 | −0.08 | −1.4 |
| Renal Cell | | | | | | | | | |
| Carcinoma | | | | | | | | | |
| Ovarian | paclitaxel protein- | 0.8816 | 47 | 23 | 0.8816 | 5.82 | 5.74 | −0.08 | −1.4 |
| Cancer | bound | | | | | | | | |
| Non-Small | nivolumab | 0.9581 | 223 | 114 | 0.9581 | 6.20 | 6.11 | −0.09 | −1.5 |
| Cell Lung | | | | | | | | | |
| Cancer | | | | | | | | | |
| Prostate | leuprolide | 0.0001 | 1046 | 480 | 0.0002 | 6.18 | 6.08 | −0.10 | −1.6 |
| Cancer | | | | | | | | | |
| Prostate | prednisone | 0.0101 | 507 | 186 | 0.0201 | 6.19 | 6.08 | −0.10 | −1.7 |
| Cancer | | | | | | | | | |
| Sarcoma | paclitaxel | 0.9417 | 35 | 22 | 0.9715 | 5.05 | 4.96 | −0.09 | −1.7 |
| Non-Small | pemetrexed | 0.0407 | 1008 | 494 | 0.0407 | 6.38 | 6.27 | −0.11 | −1.8 |
| Cell Lung | | | | | | | | | |
| Cancer | | | | | | | | | |
| Non-Small | bevacizumab | 0.6250 | 106 | 73 | 0.8030 | 6.36 | 6.25 | −0.11 | −1.8 |
| Cell Lung | | | | | | | | | |
| Cancer | | | | | | | | | |
| Esophageal | carboplatin | 0.0452 | 165 | 236 | 0.2199 | 5.90 | 5.79 | −0.11 | −1.8 |
| Cancer | | | | | | | | | |
| Gastric | leucovorin | 0.0224 | 218 | 140 | 0.0373 | 5.78 | 5.68 | −0.11 | −1.8 |
| Cancer | | | | | | | | | |
| Sarcoma | pazopanib | 0.2969 | 90 | 88 | 0.2969 | 5.13 | 5.03 | −0.10 | −1.9 |
| Breast | vinorelbine | 0.7584 | 61 | 65 | 0.7584 | 6.37 | 6.24 | −0.13 | −2.0 |
| Cancer | | | | | | | | | |
| Sarcoma | gemcitabine | 0.0591 | 186 | 131 | 0.4139 | 5.13 | 5.02 | −0.10 | −2.0 |
| Gastric | oxaliplatin | 0.0042 | 227 | 158 | 0.0106 | 5.78 | 5.66 | −0.12 | −2.1 |
| Cancer | | | | | | | | | |
| Clear Cell | cabozantinib | 0.2959 | 76 | 24 | 0.2959 | 5.56 | 5.44 | −0.12 | −2.1 |
| Renal Cell | | | | | | | | | |
| Carcinoma | | | | | | | | | |
| Breast | pegfilgrastim | 0.6449 | 61 | 61 | 0.6449 | 6.39 | 6.25 | −0.13 | −2.1 |
| Cancer | | | | | | | | | |
| Esophageal | paclitaxel | 0.0196 | 191 | 235 | 0.0785 | 5.92 | 5.79 | −0.13 | −2.1 |
| Cancer | | | | | | | | | |
| Breast | pembrolizumab | 0.2001 | 129 | 80 | 0.6104 | 6.08 | 5.95 | −0.13 | −2.2 |
| Cancer | | | | | | | | | |
| Prostate | docetaxel | 0.0130 | 462 | 223 | 0.0391 | 6.15 | 6.02 | −0.14 | −2.2 |
| Cancer | | | | | | | | | |
| Pancreatic | oxaliplatin | 0.0003 | 573 | 465 | 0.0016 | 5.91 | 5.78 | −0.13 | −2.2 |
| Cancer | | | | | | | | | |
| Biliary | irinotecan | 0.9891 | 24 | 16 | 0.9891 | 6.02 | 5.88 | −0.14 | −2.3 |
| Cancer | | | | | | | | | |
| Breast | leuprolide | 0.0270 | 92 | 95 | 0.0270 | 6.33 | 6.18 | −0.15 | −2.3 |
| Cancer | | | | | | | | | |
| Breast | paclitaxel | 0.0007 | 414 | 737 | 0.0054 | 6.19 | 6.04 | −0.15 | −2.4 |
| Cancer | | | | | | | | | |
| Breast | abemaciclib | 0.0577 | 192 | 91 | 0.0577 | 6.49 | 6.33 | −0.15 | −2.4 |
| Cancer | | | | | | | | | |
| Gastric | fluorouracil | 0.0018 | 234 | 156 | 0.0064 | 5.79 | 5.65 | −0.14 | −2.4 |
| Cancer | | | | | | | | | |
| Pancreatic | irinotecan | 0.0009 | 533 | 439 | 0.0013 | 5.91 | 5.76 | −0.14 | −2.5 |
| Cancer | | | | | | | | | |
| Pancreatic | leucovorin | 0.0002 | 601 | 460 | 0.0012 | 5.91 | 5.76 | −0.15 | −2.5 |
| Cancer | | | | | | | | | |
| Pancreatic | fluorouracil | 0.0002 | 640 | 473 | 0.0012 | 5.91 | 5.76 | −0.15 | −2.5 |
| Cancer | | | | | | | | | |
| Breast | ribociclib | 0.0829 | 122 | 63 | 0.0829 | 6.47 | 6.30 | −0.17 | −2.6 |
| Cancer | | | | | | | | | |
| Non-Small | ramucirumab | 0.1988 | 118 | 28 | 0.1988 | 6.30 | 6.13 | −0.17 | −2.8 |
| Cell Lung | | | | | | | | | |
| Cancer | | | | | | | | | |
| Sarcoma | docetaxel | 0.0589 | 159 | 122 | 0.1177 | 5.15 | 5.00 | −0.14 | −2.8 |
| Ovarian | letrozole | 0.7797 | 68 | 29 | 0.7797 | 5.90 | 5.74 | −0.17 | −2.8 |
| Cancer | | | | | | | | | |
| Non-Small | levothyroxine | 0.2366 | 119 | 29 | 0.2366 | 6.04 | 5.87 | −0.17 | −2.9 |
| Cell Lung | | | | | | | | | |
| Cancer | | | | | | | | | |
| Gastric | docetaxel | 0.0019 | 64 | 90 | 0.0113 | 5.76 | 5.59 | −0.17 | −3.0 |
| Cancer | | | | | | | | | |
| Bladder | cisplatin | 0.0117 | 221 | 224 | 0.0527 | 6.19 | 6.00 | −0.19 | −3.1 |
| Cancer | | | | | | | | | |
| Breast | doxorubicin | 0.2331 | 73 | 46 | 0.4662 | 6.31 | 6.11 | −0.20 | −3.2 |
| Cancer | liposome | | | | | | | | |
| Sarcoma | mesna | 0.0365 | 58 | 60 | 0.0365 | 5.04 | 4.88 | −0.16 | −3.2 |
| Non-Small | vinorelbine | 0.2642 | 42 | 28 | 0.5285 | 6.18 | 5.98 | −0.20 | −3.3 |
| Cell Lung | | | | | | | | | |
| Cancer | | | | | | | | | |
| Ovarian | dexamethasone | 0.0535 | 78 | 29 | 0.1337 | 5.70 | 5.48 | −0.22 | −3.9 |
| Cancer | | | | | | | | | |
| Bladder | vinblastine | 0.1701 | 31 | 53 | 0.1701 | 6.13 | 5.89 | −0.24 | −3.9 |
| Cancer | | | | | | | | | |
| Clear Cell | pazopanib | 0.0088 | 51 | 25 | 0.0176 | 5.59 | 5.37 | −0.22 | −4.0 |
| Renal Cell | | | | | | | | | |
| Carcinoma | | | | | | | | | |
| Bladder | methotrexate | 0.1023 | 33 | 53 | 0.2045 | 6.13 | 5.88 | −0.25 | −4.0 |
| Cancer | | | | | | | | | |
| Bladder | doxorubicin | 0.0910 | 32 | 52 | 0.2730 | 6.14 | 5.88 | −0.27 | −4.3 |
| Cancer | | | | | | | | | |
| Non-Small | osimertinib | 8.35E−06 | 223 | 186 | 8.4E−06 | 6.52 | 6.23 | −0.29 | −4.4 |
| Cell Lung | | | | | | | | | |
| Cancer | | | | | | | | | |
| Endometrial | bevacizumab | 0.0523 | 119 | 26 | 0.1569 | 5.81 | 5.56 | −0.26 | −4.4 |
| Cancer | | | | | | | | | |
| Pancreatic | dexamethasone | 0.0167 | 72 | 23 | 0.0834 | 6.03 | 5.75 | −0.27 | −4.5 |
| Cancer | | | | | | | | | |
| Prostate | degarelix | 1.81E−05 | 270 | 99 | 1.8E−05 | 6.23 | 5.94 | −0.29 | −4.6 |
| Cancer | | | | | | | | | |
| Clear Cell | axitinib | 0.0006 | 76 | 29 | 0.0006 | 5.72 | 5.43 | −0.29 | −5.1 |
| Renal Cell | | | | | | | | | |
| Carcinoma | | | | | | | | | |
| Biliary | paclitaxel protein- | 0.2486 | 18 | 12 | 0.8342 | 5.99 | 5.62 | −0.36 | −6.1 |
| Cancer | bound | | | | | | | | |

1. A method of treating cancer in a subject in need thereof, said method comprising:
administering to said subject a first anti-cancer agent that increases CD46 expression on the surface of a cancer cell; and wherein the first anti-cancer agent is not pomalidomide, lenalidomide, or enzalutamide, and administering to said subject a second anti-cancer agent comprising:
(i) an antibody that specifically binds CD46 or a CD46-binding fragment thereof, wherein said antibody comprises a heavy chain (HC) variable region that comprises three CDRs:
HC CDR1, HC CDR2 and HC CDR3 and a light chain (LC) variable region that comprises three CDRs: LC CDR1, LC CDR2, and LC CDR3, and wherein said HC CDR1, HC CDR2, and HC CDR3 comprise an amino acid sequence of SEQ ID NO: 1, SEQ ID NO: 2, and SEQ ID NO: 3, respectively, and said LC CDR1, LC CDR2, and LC CDR3 comprise an amino acid sequence of SEQ ID NO: 64, SEQ ID NO: 65, and SEQ ID NO: 66, respectively; and
(ii) a cytotoxic effector coupled to said antibody, wherein said cytotoxic effector comprises monomethylauristatin E (MMAE).

2. The method of claim 1, wherein said first anti-cancer agent comprises a drug or prodrug thereof, a first antibody, a peptide, a protein, a liposome containing the drug or prodrug thereof, a radionuclide, a viral particle, or a chelate.

3. The method of claim 1, wherein said first anti-cancer agent comprises a drug.

4. The method of claim 2, wherein said drug comprises an anti-cancer drug, a chemotherapeutic agent, a microtubule inhibitor, a DNA-damaging agent, or a polymerase inhibitor.

5. The method of claim 1, wherein said first anti-cancer agent comprises an immunotherapy.

6. (canceled)

7. The method of claim 1, wherein the first anti-cancer agent is not a pomalidomide analog, a lenalidomide analog, or an enzalutamide analog.

8-9. (canceled)

10. The method of claim 1, wherein said second anti-cancer agent comprises a constant region of an IgG heavy chain.

11. The method of claim 1, wherein said second anti-cancer agent comprises a constant region of an IgG1 heavy chain.

12. The method of claim 1, wherein said second anti-cancer agent comprises a single chain variable fragment (scFv), a single domain antibody (sdA), a Fab, or a Fab′.

13. The method of claim 1, wherein the antibody that specifically binds CD46 or the CD46-binding fragment thereof binds domain 1 or 2 of CD46.

14-23. (canceled)

24. The method of claim 1, wherein a ratio of said cytotic effector to said antibody that specifically binds CD46 or a CD46-binding fragment thereof is about 2:1, 4:1, 6:1, or 8:1.

25. The method of claim 1, wherein a ratio of said cytotic effector to said antibody that specifically binds CD46 or a CD46-binding fragment thereof is about 4:1.

26. The method of claim 1, wherein said cytotoxic effector is conjugated to said antibody that specifically binds CD46 via a linker.

27. The method of claim 1, wherein said linker comprises a peptide, small molecule, or a combination thereof

28. The method of claim 1, wherein said linker comprises maleimidocaproyl-valine-citrulline-para-amino benzyloxycarbonyl (mc-vc-PAB).

29. The method of claim 1, wherein said cancer comprises ovarian cancer, colorectal cancer, breast cancer, lung cancer, kidney cancer, pancreatic cancer, mesothelioma, lymphoma, liver cancer, urothelial cancer, stomach cancer, glioblastoma multiforme, glioma, neuroblastoma, head and neck or cervical cancer.

30-44. (canceled)

45. The method of claim 1, wherein said first anti-cancer agent and/or said second anti-cancer agent is administered to said subject orally, nasally, rectally, intraperitoneally, subcutaneously, transcutaneously, intramuscularly, or intravenously.

46. The method of claim 1, wherein said first anti-cancer agent and/or said second anti-cancer agent is administered to said subject via intravenous infusion.

47. The method of claim 1, wherein said first anti-cancer agent and/or second anti-cancer agent is administered in an effective amount.

48. The method of claim 47, wherein said effective amount of the second anti-cancer agent comprises a dose from about 1.0 to 5.0 mg/kg.

49. The method of claim 48, wherein said dose is about 1.2 mg/kg.

50. The method of claim 48, wherein said dose is about 1.8 mg/kg.

51. The method of claim 48, wherein said dose is about 2.4 mg/kg.

52. The method of claim 48, wherein said dose is about 3.2 mg/kg.

53. The method of claim 48, wherein said dose is administered every 2-4 weeks.

54. The method of claim 48, wherein said dose is administered about every 3 weeks.

55. The method of claim 47, wherein said effective amount of the first anti-cancer agent increases a response of the cancer cell to the second anti-cancer agent.

56. The method of claim 1, wherein said increased CD46 expression on the surface of the cancer cell is relative to a control measurement or relative to a baseline measurement.

57. The method of claim 1, wherein said first anti-cancer agent enhances an antibody-dependent cellular cytotoxicty activity of the second anti-cancer agent on the cancer cell.

58. The method of claim 47, wherein said effective amount of the first anti-cancer agent or of the second anti-cancer agent is lower than an effective amount in a method not including administration of both the first anti-cancer agent and second anti-cancer agent.

59. The method of claim 1, wherein a first dose of said first anti-cancer agent is administered before a first dose of said second anti-cancer agent.

60-114. (canceled)


SEQUENCE LISTING (206 sequences)

SEQ ID NO: 1 - protein, 8 aa, Artificial Sequence
  Description of Artificial Sequence Synthetic peptide
GLTVNNYA

SEQ ID NO: 2 - protein, 8 aa, Artificial Sequence
  Description of Artificial Sequence Synthetic peptide
ISYDGNNK

SEQ ID NO: 3 - protein, 9 aa, Artificial Sequence
  Description of Artificial Sequence Synthetic peptide
AKGGGYFDL

SEQ ID NO: 4 - protein, 8 aa, Artificial Sequence
  Description of Artificial Sequence Synthetic peptide
GFTVNNYA

SEQ ID NO: 5 - protein, 8 aa, Artificial Sequence
  Description of Artificial Sequence Synthetic peptide
ISYDGNNK

SEQ ID NO: 6 - protein, 9 aa, Artificial Sequence
  Description of Artificial Sequence Synthetic peptide
AKGGGYFDL

SEQ ID NO: 7 - protein, 8 aa, Artificial Sequence
  Description of Artificial Sequence Synthetic peptide
GFTVNNYA

SEQ ID NO: 8 - protein, 8 aa, Artificial Sequence
  Description of Artificial Sequence Synthetic peptide
ISYDGNNK

SEQ ID NO: 9 - protein, 9 aa, Artificial Sequence
  Description of Artificial Sequence Synthetic peptide
AKGGGYFDL

SEQ ID NO: 10 - protein, 8 aa, Artificial Sequence
  Description of Artificial Sequence Synthetic peptide
GFTFSSYA

SEQ ID NO: 11 - protein, 8 aa, Artificial Sequence
  Description of Artificial Sequence Synthetic peptide
IRSDGSKK

SEQ ID NO: 12 - protein, 9 aa, Artificial Sequence
  Description of Artificial Sequence Synthetic peptide
ARHGNYFDS

SEQ ID NO: 13 - protein, 8 aa, Artificial Sequence
  Description of Artificial Sequence Synthetic peptide
GFTFSTYG

SEQ ID NO: 14 - protein, 8 aa, Artificial Sequence
  Description of Artificial Sequence Synthetic peptide
ISYDGDEK

SEQ ID NO: 15 - protein, 13 aa, Artificial Sequence
  Description of Artificial Sequence Synthetic peptide
AKASGYGMGILDY

SEQ ID NO: 16 - protein, 8 aa, Artificial Sequence
  Description of Artificial Sequence Synthetic peptide
GFTFSDYY

SEQ ID NO: 17 - protein, 8 aa, Artificial Sequence
  Description of Artificial Sequence Synthetic peptide
ISSSGSTI

SEQ ID NO: 18 - protein, 14 aa, Artificial Sequence
  Description of Artificial Sequence Synthetic peptide
ARDYGRIAAAGRRY

SEQ ID NO: 19 - protein, 8 aa, Artificial Sequence
  Description of Artificial Sequence Synthetic peptide
GFTFSDYY

SEQ ID NO: 20 - protein, 8 aa, Artificial Sequence
  Description of Artificial Sequence Synthetic peptide
ISSSGSTI

SEQ ID NO: 21 - protein, 14 aa, Artificial Sequence
  Description of Artificial Sequence Synthetic peptide
ARDYGRIAAAGRHY

SEQ ID NO: 22 - protein, 8 aa, Artificial Sequence
  Description of Artificial Sequence Synthetic peptide
GFTFSDYY

SEQ ID NO: 23 - protein, 8 aa, Artificial Sequence
  Description of Artificial Sequence Synthetic peptide
ISSSGSTI

SEQ ID NO: 24 - protein, 14 aa, Artificial Sequence
  Description of Artificial Sequence Synthetic peptide
ARDYGRIAAAGRHY

SEQ ID NO: 25 - protein, 8 aa, Artificial Sequence
  Description of Artificial Sequence Synthetic peptide
GFTFSSYW

SEQ ID NO: 26 - protein, 8 aa, Artificial Sequence
  Description of Artificial Sequence Synthetic peptide
IKQDGSEK

SEQ ID NO: 27 - protein, 20 aa, Artificial Sequence
  Description of Artificial Sequence Synthetic peptide
AKDVGSTAINYVRAYTWFDP

SEQ ID NO: 28 - protein, 8 aa, Artificial Sequence
  Description of Artificial Sequence Synthetic peptide
GFTFSNYA

SEQ ID NO: 29 - protein, 8 aa, Artificial Sequence
  Description of Artificial Sequence Synthetic peptide
ISGSGSST

SEQ ID NO: 30 - protein, 15 aa, Artificial Sequence
  Description of Artificial Sequence Synthetic peptide
AQGLYSSGWANWFDP

SEQ ID NO: 31 - protein, 8 aa, Artificial Sequence
  Description of Artificial Sequence Synthetic peptide
GFTFSSYG

SEQ ID NO: 32 - protein, 8 aa, Artificial Sequence
  Description of Artificial Sequence Synthetic peptide
ISYDGSNK

SEQ ID NO: 33 - protein, 16 aa, Artificial Sequence
  Description of Artificial Sequence Synthetic peptide
AKVMGLAAAGLDAFDI

SEQ ID NO: 34 - protein, 8 aa, Artificial Sequence
  Description of Artificial Sequence Synthetic peptide
GFTFSSYA

SEQ ID NO: 35 - protein, 8 aa, Artificial Sequence
  Description of Artificial Sequence Synthetic peptide
ISYDGSNK

SEQ ID NO: 36 - protein, 10 aa, Artificial Sequence
  Description of Artificial Sequence Synthetic peptide
GRESSGSPGV

SEQ ID NO: 37 - protein, 8 aa, Artificial Sequence
  Description of Artificial Sequence Synthetic peptide
GFTVSSNY

SEQ ID NO: 38 - protein, 7 aa, Artificial Sequence
  Description of Artificial Sequence Synthetic peptide
IYTDGST

SEQ ID NO: 39 - protein, 16 aa, Artificial Sequence
  Description of Artificial Sequence Synthetic peptide
ARDRGTSGYDWAWFDL

SEQ ID NO: 40 - protein, 8 aa, Artificial Sequence
  Description of Artificial Sequence Synthetic peptide
GFTFSSYA

SEQ ID NO: 41 - protein, 8 aa, Artificial Sequence
  Description of Artificial Sequence Synthetic peptide
ISGSGGST

SEQ ID NO: 42 - protein, 16 aa, Artificial Sequence
  Description of Artificial Sequence Synthetic peptide
AKDRYYYGSGKDAFDI

SEQ ID NO: 43 - protein, 8 aa, Artificial Sequence
  Description of Artificial Sequence Synthetic peptide
GFTFSNYW

SEQ ID NO: 44 - protein, 8 aa, Artificial Sequence
  Description of Artificial Sequence Synthetic peptide
VRQDGGQK

SEQ ID NO: 45 - protein, 11 aa, Artificial Sequence
  Description of Artificial Sequence Synthetic peptide
VSQRNSGEHDY

SEQ ID NO: 46 - protein, 8 aa, Artificial Sequence
  Description of Artificial Sequence Synthetic peptide
GFTFSDYY

SEQ ID NO: 47 - protein, 8 aa, Artificial Sequence
  Description of Artificial Sequence Synthetic peptide
ISSSGSTI

SEQ ID NO: 48 - protein, 14 aa, Artificial Sequence
  Description of Artificial Sequence Synthetic peptide
ARDYGRIAAAGRHY

SEQ ID NO: 49 - protein, 8 aa, Artificial Sequence
  Description of Artificial Sequence Synthetic peptide
GFTFSDYY

SEQ ID NO: 50 - protein, 8 aa, Artificial Sequence
  Description of Artificial Sequence Synthetic peptide
ISSSGSTI

SEQ ID NO: 51 - protein, 14 aa, Artificial Sequence
  Description of Artificial Sequence Synthetic peptide
ARDYGRIAAAGRNY

SEQ ID NO: 52 - protein, 8 aa, Artificial Sequence
  Description of Artificial Sequence Synthetic peptide
GFTFSDYY

SEQ ID NO: 53 - protein, 8 aa, Artificial Sequence
  Description of Artificial Sequence Synthetic peptide
ISSSGSTI

SEQ ID NO: 54 - protein, 14 aa, Artificial Sequence
  Description of Artificial Sequence Synthetic peptide
ARDYGRIAAAGRHY

SEQ ID NO: 55 - protein, 8 aa, Artificial Sequence
  Description of Artificial Sequence Synthetic peptide
GFTFSDYY

SEQ ID NO: 56 - protein, 8 aa, Artificial Sequence
  Description of Artificial Sequence Synthetic peptide
ISSSGSSI

SEQ ID NO: 57 - protein, 14 aa, Artificial Sequence
  Description of Artificial Sequence Synthetic peptide
ARDITDVVGVSFDY

SEQ ID NO: 58 - protein, 8 aa, Artificial Sequence
  Description of Artificial Sequence Synthetic peptide
GFTFSSYG

SEQ ID NO: 59 - protein, 8 aa, Artificial Sequence
  Description of Artificial Sequence Synthetic peptide
ISYDGSNK

SEQ ID NO: 60 - protein, 16 aa, Artificial Sequence
  Description of Artificial Sequence Synthetic peptide
AKVMGLAAAGLDAFDI

SEQ ID NO: 61 - protein, 8 aa, Artificial Sequence
  Description of Artificial Sequence Synthetic peptide
GFTFSSFG

SEQ ID NO: 62 - protein, 8 aa, Artificial Sequence
  Description of Artificial Sequence Synthetic peptide
ISYDGSNQ

SEQ ID NO: 63 - protein, 16 aa, Artificial Sequence
  Description of Artificial Sequence Synthetic peptide
GSRPGGGYASGSTVAY

SEQ ID NO: 64 - protein, 9 aa, Artificial Sequence
  Description of Artificial Sequence Synthetic peptide
SSNIGAGYD

SEQ ID NO: 65 - protein, 3 aa, Artificial Sequence
  Description of Artificial Sequence Synthetic peptide
GNN

SEQ ID NO: 66 - protein, 9 aa, Artificial Sequence
  Description of Artificial Sequence Synthetic peptide
SSYTSGTWL

SEQ ID NO: 67 - protein, 9 aa, Artificial Sequence
  Description of Artificial Sequence Synthetic peptide
SSNIGAGYD

SEQ ID NO: 68 - protein, 3 aa, Artificial Sequence
  Description of Artificial Sequence Synthetic peptide
GNN

SEQ ID NO: 69 - protein, 9 aa, Artificial Sequence
  Description of Artificial Sequence Synthetic peptide
SSYTSGTWL

SEQ ID NO: 70 - protein, 9 aa, Artificial Sequence
  Description of Artificial Sequence Synthetic peptide
SSNIGAGYD

SEQ ID NO: 71 - protein, 3 aa, Artificial Sequence
  Description of Artificial Sequence Synthetic peptide
GDN

SEQ ID NO: 72 - protein, 9 aa, Artificial Sequence
  Description of Artificial Sequence Synthetic peptide
SSYTSGTWL

SEQ ID NO: 73 - protein, 6 aa, Artificial Sequence
  Description of Artificial Sequence Synthetic peptide
QGISSY

SEQ ID NO: 74 - protein, 3 aa, Artificial Sequence
  Description of Artificial Sequence Synthetic peptide
AAS

SEQ ID NO: 75 - protein, 9 aa, Artificial Sequence
  Description of Artificial Sequence Synthetic peptide
QQLASYPLT

SEQ ID NO: 76 - protein, 6 aa, Artificial Sequence
  Description of Artificial Sequence Synthetic peptide
SLRSYY

SEQ ID NO: 77 - protein, 3 aa, Artificial Sequence
  Description of Artificial Sequence Synthetic peptide
GQN

SEQ ID NO: 78 - protein, 12 aa, Artificial Sequence
  Description of Artificial Sequence Synthetic peptide
HSRDSSGTHLRV

SEQ ID NO: 79 - protein, 8 aa, Artificial Sequence
  Description of Artificial Sequence Synthetic peptide
TSNIGSNH

SEQ ID NO: 80 - protein, 3 aa, Artificial Sequence
  Description of Artificial Sequence Synthetic peptide
RNN

SEQ ID NO: 81 - protein, 11 aa, Artificial Sequence
  Description of Artificial Sequence Synthetic peptide
ATWDDSLSGEV

SEQ ID NO: 82 - protein, 6 aa, Artificial Sequence
  Description of Artificial Sequence Synthetic peptide
SLRSYY

SEQ ID NO: 83 - protein, 3 aa, Artificial Sequence
  Description of Artificial Sequence Synthetic peptide
GKN

SEQ ID NO: 84 - protein, 12 aa, Artificial Sequence
  Description of Artificial Sequence Synthetic peptide
NSRDSSGTHLEV

SEQ ID NO: 85 - protein, 6 aa, Artificial Sequence
  Description of Artificial Sequence Synthetic peptide
TLSTYY

SEQ ID NO: 86 - protein, 3 aa, Artificial Sequence
  Description of Artificial Sequence Synthetic peptide
GKN

SEQ ID NO: 87 - protein, 10 aa, Artificial Sequence
  Description of Artificial Sequence Synthetic peptide
HSRDISGNYL

SEQ ID NO: 88 - protein, 8 aa, Artificial Sequence
  Description of Artificial Sequence Synthetic peptide
SSNIGSNT

SEQ ID NO: 89 - protein, 3 aa, Artificial Sequence
  Description of Artificial Sequence Synthetic peptide
SNN

SEQ ID NO: 90 - protein, 11 aa, Artificial Sequence
  Description of Artificial Sequence Synthetic peptide
AAWDDSLNVYV

SEQ ID NO: 91 - protein, 6 aa, Artificial Sequence
  Description of Artificial Sequence Synthetic peptide
RDIRND

SEQ ID NO: 92 - protein, 3 aa, Artificial Sequence
  Description of Artificial Sequence Synthetic peptide
GAS

SEQ ID NO: 93 - protein, 19 aa, Artificial Sequence
  Description of Artificial Sequence Synthetic peptide
HRLNSYPLTFGGGTKVDIK

SEQ ID NO: 94 - protein, 9 aa, Artificial Sequence
  Description of Artificial Sequence Synthetic peptide
SSDVGGYNY

SEQ ID NO: 95 - protein, 3 aa, Artificial Sequence
  Description of Artificial Sequence Synthetic peptide
DVS

SEQ ID NO: 96 - protein, 11 aa, Artificial Sequence
  Description of Artificial Sequence Synthetic peptide
SSYTSSSTPWV

SEQ ID NO: 97 - protein, 6 aa, Artificial Sequence
  Description of Artificial Sequence Synthetic peptide
SLRSYY

SEQ ID NO: 98 - protein, 3 aa, Artificial Sequence
  Description of Artificial Sequence Synthetic peptide
GKN

SEQ ID NO: 99 - protein, 9 aa, Artificial Sequence
  Description of Artificial Sequence Synthetic peptide
NSRDSSGNQ

SEQ ID NO: 100 - protein, 6 aa, Artificial Sequence
  Description of Artificial Sequence Synthetic peptide
SLRTYY

SEQ ID NO: 101 - protein, 3 aa, Artificial Sequence
  Description of Artificial Sequence Synthetic peptide
GKN

SEQ ID NO: 102 - protein, 11 aa, Artificial Sequence
  Description of Artificial Sequence Synthetic peptide
NSRDSSGNHVV

SEQ ID NO: 103 - protein, 9 aa, Artificial Sequence
  Description of Artificial Sequence Synthetic peptide
GSDVGSYNY

SEQ ID NO: 104 - protein, 3 aa, Artificial Sequence
  Description of Artificial Sequence Synthetic peptide
EVS

SEQ ID NO: 105 - protein, 10 aa, Artificial Sequence
  Description of Artificial Sequence Synthetic peptide
SSYTTSSTLV

SEQ ID NO: 106 - protein, 6 aa, Artificial Sequence
  Description of Artificial Sequence Synthetic peptide
SLRSYY

SEQ ID NO: 107 - protein, 3 aa, Artificial Sequence
  Description of Artificial Sequence Synthetic peptide
GEN

SEQ ID NO: 108 - protein, 11 aa, Artificial Sequence
  Description of Artificial Sequence Synthetic peptide
NSWDSSGNHVV

SEQ ID NO: 109 - protein, 6 aa, Artificial Sequence
  Description of Artificial Sequence Synthetic peptide
QSISSY

SEQ ID NO: 110 - protein, 3 aa, Artificial Sequence
  Description of Artificial Sequence Synthetic peptide
AAS

SEQ ID NO: 111 - protein, 9 aa, Artificial Sequence
  Description of Artificial Sequence Synthetic peptide
QQSYSTPRT

SEQ ID NO: 112 - protein, 11 aa, Artificial Sequence
  Description of Artificial Sequence Synthetic peptide
QSLLHSNGYDY

SEQ ID NO: 113 - protein, 3 aa, Artificial Sequence
  Description of Artificial Sequence Synthetic peptide
LGS

SEQ ID NO: 114 - protein, 8 aa, Artificial Sequence
  Description of Artificial Sequence Synthetic peptide
MQGLQTPS

SEQ ID NO: 115 - protein, 6 aa, Artificial Sequence
  Description of Artificial Sequence Synthetic peptide
SLRSYY

SEQ ID NO: 116 - protein, 3 aa, Artificial Sequence
  Description of Artificial Sequence Synthetic peptide
GKN

SEQ ID NO: 117 - protein, 12 aa, Artificial Sequence
  Description of Artificial Sequence Synthetic peptide
NSRDSSSTHRGV

SEQ ID NO: 118 - protein, 6 aa, Artificial Sequence
  Description of Artificial Sequence Synthetic peptide
RSISTY

SEQ ID NO: 119 - protein, 3 aa, Artificial Sequence
  Description of Artificial Sequence Synthetic peptide
DAS

SEQ ID NO: 120 - protein, 9 aa, Artificial Sequence
  Description of Artificial Sequence Synthetic peptide
QQSYNPPWT

SEQ ID NO: 121 - protein, 9 aa, Artificial Sequence
  Description of Artificial Sequence Synthetic peptide
SSDVGGYNY

SEQ ID NO: 122 - protein, 3 aa, Artificial Sequence
  Description of Artificial Sequence Synthetic peptide
DVS

SEQ ID NO: 123 - protein, 11 aa, Artificial Sequence
  Description of Artificial Sequence Synthetic peptide
SSYTSSSDPWV

SEQ ID NO: 124 - protein, 6 aa, Artificial Sequence
  Description of Artificial Sequence Synthetic peptide
QPISTY

SEQ ID NO: 125 - protein, 3 aa, Artificial Sequence
  Description of Artificial Sequence Synthetic peptide
GAS

SEQ ID NO: 126 - protein, 19 aa, Artificial Sequence
  Description of Artificial Sequence Synthetic peptide
QQSYSSLLTFGDGTKVEIK

SEQ ID NO: 127 - protein, 116 aa, Artificial Sequence
  Description of Artificial Sequence Synthetic polypeptide
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 128 - protein, 116 aa, Artificial Sequence
  Description of Artificial Sequence Synthetic polypeptide
QVQLVQSGGGVVQPGRSLRLACAASGFTVNNYAMHWVRQAPGKGLEWVAVISYDGNNKYYADSVKGRFTISRDNSKNTLYLQMNSLRAEDTAVYYCAKGGGYFDLWGRGTLVTVSS

SEQ ID NO: 129 - protein, 116 aa, Artificial Sequence; identical to SEQ ID NO: 128
  Description of Artificial Sequence Synthetic polypeptide

SEQ ID NO: 130 - protein, 116 aa, Artificial Sequence
  Description of Artificial Sequence Synthetic polypeptide
QVQLQQSGGGVVQPGRSLRLSCAASGFTFSSYAMHWVRQAPGKGLEWVAFIRSDGSKKYYADSVKGRFTISRDNSKNTLYLQMNSLRAEDTAVYYCARHGNYFDSWGQGTLVTVSS

SEQ ID NO: 131 - protein, 120 aa, Artificial Sequence
  Description of Artificial Sequence Synthetic polypeptide
QVQLVESGGGVVQPGRSLRLSCAASGFTFSTYGMHWVRQAPGKGLEWLSFISYDGDEKYYADSVKGRFTISRDNSKNTLYLQMNSLRAEDTAVYWCAKASGYGMGILDYWGQGTLVTVSS

SEQ ID NO: 132 - protein, 121 aa, Artificial Sequence
  Description of Artificial Sequence Synthetic polypeptide
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRRYWGQGTLVTVSS

SEQ ID NO: 133 - protein, 121 aa, Artificial Sequence
  Description of Artificial Sequence Synthetic polypeptide
QVQLQESGGGVVRPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRHYWGQGTLVTVSS

SEQ ID NO: 134 - protein, 121 aa, Artificial Sequence
  Description of Artificial Sequence Synthetic polypeptide
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRHYWGQGTLVTVSS

SEQ ID NO: 135 - protein, 127 aa, Artificial Sequence
  Description of Artificial Sequence Synthetic polypeptide
QVQLQESGGGLVQPGGSLRLSCAASGFTFSSYWMSWVRQAPGKGLEWVADIKQDGSEKYYVDSVKGRFTISGDNAKNSLYLQMNSLRAEDTAVYYCAKDVGSTAINYVRAYTWFDPWGQGTLVTVSS

SEQ ID NO: 136 - protein, 122 aa, Artificial Sequence
  Description of Artificial Sequence Synthetic polypeptide
QVQLQESGGGLVQPGGSLRLSCAASGFTFSNYAMSWVRQAPGKGLEWVSTISGSGSSTFYVDSVKGRFTISRDNSKNTLYLQMNSLRAEDTAVYYCAQGLYSSGWANWFDPRGQGTLVTVSS

SEQ ID NO: 137 - protein, 123 aa, Artificial Sequence
  Description of Artificial Sequence Synthetic polypeptide
QVQLQESGGGVVQPGRSLRLSCAASGFTFSSYGMHWVRQAPGKGLEWVAVISYDGSNKYYADSVKGRFTISRDNSKNTLYLQMNSLRAEDTAVYYCAKVMGLAAAGLDAFDIWGQGTTVTVSS

SEQ ID NO: 138 - protein, 117 aa, Artificial Sequence
  Description of Artificial Sequence Synthetic polypeptide
QVQLVQSGGGVVQPGRSLRLSCAASGFTFSSYAMHWVRQAPGKGLEWVAVISYDGSNKYYADSVKGRFTISRDTSTNTLYLQMNSLRADDTAVYYCGRESSGSPGVWGQGTTVTVSS

SEQ ID NO: 139 - protein, 122 aa, Artificial Sequence
  Description of Artificial Sequence Synthetic polypeptide
QVQLVESGGGLIQPGGSLRLSCAASGFTVSSNYMSWVRQAPGKGLEWVSVIYTDGSTYYADSVKGRFTISRDNSKNTLYLQMNSLRAEDTAIYYCARDRGTSGYDWAWFDLWGQGTLVTVSS

SEQ ID NO: 140 - protein, 123 aa, Artificial Sequence
  Description of Artificial Sequence Synthetic polypeptide
QVQLQESGGGLVQPGGSLRLSCAASGFTFSSYAMSWVRQAPGKGLEWVSAISGSGGSTYYADSVKGRFTISRDNSKNTLYMQMNSLRAEDTAVYYCAKDRYYYGSGKDAFDIWGRGTMVTVSS

SEQ ID NO: 141 - protein, 118 aa, Artificial Sequence
  Description of Artificial Sequence Synthetic polypeptide
QVQLVESGGGLVQPGGSLGLSCAASGFTFSNYWMSWVRQAPGKGLEWVANVRQDGGQKYYVDSVKGRFTISRDNAKNSLYLQMNSLRTEDTAVYFCVSQRNSGEHDYWGQGTLVTVSS

SEQ ID NO: 142 - protein, 121 aa, Artificial Sequence; identical to SEQ ID NO: 134
  Description of Artificial Sequence Synthetic polypeptide

SEQ ID NO: 143 - protein, 121 aa, Artificial Sequence
  Description of Artificial Sequence Synthetic polypeptide
EVQLVESGGGLVQPGGSLRLSCAASGFTFSDYYMSWIRQAPGKGLEWVSYISSSGSTIYYADSVKGRFTISRDNSKNTLYLQMNSLRAEDTAVYYCARDYGRIAAAGRNYWGQGTLVTVSS

SEQ ID NO: 144 - protein, 121 aa, Artificial Sequence; identical to SEQ ID NO: 133
  Description of Artificial Sequence Synthetic polypeptide

SEQ ID NO: 145 - protein, 121 aa, Artificial Sequence
  Description of Artificial Sequence Synthetic polypeptide
EVQLVESGGGLVKPGGSLRLSCAASGFTFSDYYMSWIRQAPGKGLEWVSYISSSGSSIYYADSVKGRFTISRDNAKNSLYLQMNSLKAEDTAVYYCARDITDVVGVSFDYWGQGTLVTVSS

SEQ ID NO: 146 - protein, 123 aa, Artificial Sequence
  Description of Artificial Sequence Synthetic polypeptide
EVQLVESGGGVVQPGRSLRLSCAASGFTFSSYGMHWVRQAPGKGLEWVAVISYDGSNKYYADSVKGRFTISRDNSKNTLYLQMNSLRAEDTAEYYCAKVMGLAAAGLDAFDIWGQGTLVTVSS

SEQ ID NO: 147 - protein, 123 aa, Artificial Sequence
  Description of Artificial Sequence Synthetic polypeptide
EVQLVESGGGVVQPGRSLRLSCAASGFTFSSFGMHWVRRAPGKGLEWVAVISYDGSNQYYADSVKGRFTISRDNSKNTLYLQMNSLRAEDTAVYYCGSRPGGGYASGSTVAYWGQGTLVTVSS

SEQ ID NO: 148 - protein, 109 aa, Artificial Sequence
  Description of Artificial Sequence Synthetic polypeptide
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVL

SEQ ID NO: 149 - protein, 109 aa, Artificial Sequence; identical to SEQ ID NO: 148
  Description of Artificial Sequence Synthetic polypeptide

SEQ ID NO: 150 - protein, 109 aa, Artificial Sequence
  Description of Artificial Sequence Synthetic polypeptide
QSVLTQPPSVSGAPGQRVTISCTGSSSNIGAGYDVHWYQQLPGTAPKLLIYGDNNRPSGVPDRFSGSKSGTSASLAITGLQAEDEADYYCSSYTSGTWLFGGGTKLTVL

SEQ ID NO: 151 - protein, 107 aa, Artificial Sequence
  Description of Artificial Sequence Synthetic polypeptide
DIQMTQSPSFLSASVGDRVTITCRASQGISSYLAWYQQKPGKAPKLLIYAASTLQSGVPSSFSGSGSGTEFTLTISSLQPEDFATYYCQQLASYPLTFGGGTKVDIK

SEQ ID NO: 152 - protein, 109 aa, Artificial Sequence
  Description of Artificial Sequence Synthetic polypeptide
SSELTQDPAVSVALGQTVRITCQGDSLRSYYVSWFQQKPGQAPVFVMYGQNNRPSGISERFSGSSSGNTASLIITGAQAEDEADYYCHSRDSSGTHLRVFGGGTKLTVL

SEQ ID NO: 153 - protein, 110 aa, Artificial Sequence
  Description of Artificial Sequence Synthetic polypeptide
QSALTQPPSASATPGQRVTISCSGRTSNIGSNHVYWYQQLPGTAPKLLIYRNNQRPSGVPDRFSGSKSGTSASLAISGLRSEDEADYYCATWDDSLSGEVFGGGTKLTVL

SEQ ID NO: 154 - protein, 109 aa, Artificial Sequence
  Description of Artificial Sequence Synthetic polypeptide
SSELTQDPAVSVALGQTVRITCQGDSLRSYYASWYQQKPGQAPVLVIYGKNNRPSGIPDRFSGSSSGNTASLTITGAQAEDEADYYCNSRDSSGTHLEVFGGGTKVTVL

SEQ ID NO: 155 - protein, 107 aa, Artificial Sequence
  Description of Artificial Sequence Synthetic polypeptide
SSELTQDPAVSVALGQTVRITCQGDTLSTYYANWYQQKPGQAPVLVIYGKNNRPSGIPDRFSGSSSGNTASLTITGAQAEDEADYYCHSRDISGNYLFASGTKLTVL

SEQ ID NO: 156 - protein, 110 aa, Artificial Sequence
  Description of Artificial Sequence Synthetic polypeptide
QSVLTQPPSASGTPGQRVTISCSGSSSNIGSNTVNWSRQLPGTAPKLLIYSNNQRPSGVPDRFSGSKSGTSASLAISGLQSEDEADYYCAAWDDSLNVYVFGTGTKVTVL

SEQ ID NO: 157 - protein, 107 aa, Artificial Sequence
  Description of Artificial Sequence Synthetic polypeptide
KIVLTQSPSSLSASVGDTVTIACRASRDIRNDLAWYQQKPGKAPKLLIYGASSLQSGVPSRFSGSGSGTEFILTISSLQPEDFATYYCHRLNSYPLTFGGGTKVDIK

SEQ ID NO: 158 - protein, 111 aa, Artificial Sequence
  Description of Artificial Sequence Synthetic polypeptide
NFMLTQPASLSGSPGQSITISCTGTSSDVGGYNYVSWYQQHPGYAPKLMIYDVSNRPSGVSNRFSGSKSGNTASLTISGLQAEDEADYYCSSYTSSSTPWVFGGGTKLTVL

SEQ ID NO: 159 - protein, 106 aa, Artificial Sequence
  Description of Artificial Sequence Synthetic polypeptide
SYVLTQDPAVSVALGQTVRITCQGDSLRSYYASWYQQKPGQAPVLVIYGKNNRPSGIPDRFSGSSSGNTASLTITGAQAEDEADYYCNSRDSSGNQFGGGTKLTVL

SEQ ID NO: 160 - protein, 108 aa, Artificial Sequence
  Description of Artificial Sequence Synthetic polypeptide
SSELTQDPAVSVALGQTVRITCQGDSLRTYYASWYQQRPGQAPILVLYGKNNRPSGIPDRFSGSSSGNTASLTITGAQAEDEADYYCNSRDSSGNHVVFGGGTKLTVL

SEQ ID NO: 161 - protein, 110 aa, Artificial Sequence
  Description of Artificial Sequence Synthetic polypeptide
QSVLTQPASVSGSPGQSITISCTGTGSDVGSYNYVSWYQQNPGKAPKLMIYEVSNRPSGVSNRFSGSKSGNTASLTISGLQAEDEADYYCSSYTTSSTLVFGGGTKVTVL

SEQ ID NO: 162 - protein, 107 aa, Artificial Sequence
  Description of Artificial Sequence Synthetic polypeptide
SELTQDPAVSVALGQTVRITCQGDSLRSYYASWYQQKPGQAPVLVIYGENSRPSGIPDRFSGSSSGNTASLTITGAQAEDEADYYCNSWDSSGNHVVFGGGTKLTVL

SEQ ID NO: 163 - protein, 107 aa, Artificial Sequence
  Description of Artificial Sequence Synthetic polypeptide
AIRMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKLEIK

SEQ ID NO: 164 - protein, 111 aa, Artificial Sequence
  Description of Artificial Sequence Synthetic polypeptide
DIVMTQSPLSLPVTPGEPASISCRSSQSLLHSNGYDYLDWYLQKPGQSPQLLIYLGSNRASGVPDRFSGSGSGTDFTLKISRVETEDVGIYYCMQGLQTPSFGQGTKLEIK

SEQ ID NO: 165 - protein, 109 aa, Artificial Sequence
  Description of Artificial Sequence Synthetic polypeptide
SSELTQDPAVSVALGQTVRITCQGDSLRSYYASWYQQKPGQAPVPVIYGKNNRPSGIPDRFSGSSSGNTASLTITGAQAEDEADYYCNSRDSSSTHRGVFGGGTKLTVL

SEQ ID NO: 166 - protein, 107 aa, Artificial Sequence
  Description of Artificial Sequence Synthetic polypeptide
DIQLTQSPSSLSASVGDRVTITCRASRSISTYLSWYQQKPGKAPKLLIYDASRLQNGVPSRFSGSGSDTDFTLTISSLQPEDFATYFCQQSYNPPWTFGQGTKLEIK

SEQ ID NO: 167 - protein, 111 aa, Artificial Sequence
  Description of Artificial Sequence Synthetic polypeptide
QSALTQPASVSGSPGQSITISCTGTSSDVGGYNYVSWYQQHPGKAPKLMIYDVSNRPSGVSNRFSGSKSGNTASLTISGLQAEDEAYYYCSSYTSSSDPWVFGGGTQLTVL

SEQ ID NO: 168 - protein, 107 aa, Artificial Sequence
  Description of Artificial Sequence Synthetic polypeptide
NIQMTQSPSSLSASVGDRVTITCRAGQPISTYVNWYQHKPGKAPKLLIYGASNLQSGVPSRFSGGGSATDFTLTISSLQPEDFATYYCQQSYSSLLTFGDGTKVEIK

SEQ ID NO: 169 - protein, 446 aa, Artificial Sequence
  Description of Artificial Sequence Synthetic polypeptide
QVQLVQSGGGVVQPGRSLRLACAASGLTVNNYAMHWVRQAPGKGLEWVAVISYDGNNKYYADSVKGRFTISRDNSKNTLYLQMNSLRAEDTAVYYCAKGGGYFDLWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 170 - protein, 215 aa, Artificial Sequence
  Description of Artificial Sequence Synthetic polypeptide
QSVLTQPPSVSGAPGQRVTISCTGSSSNIGAGYDVHWYQQLPGTAPKLLIYGNNNRPSGVPDRFSGSKSGTSASLAITGLQAEDEADYYCSSYTSGTWLFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 171 - protein, 242 aa, Artificial Sequence
  Description of Artificial Sequence Synthetic polypeptide
QVQLQESGGGLVKPGGPLRLSCAASGFTFSSYGMYWVRQAPGKGLEWVSTLSRSGSGTYYADSVKGRFTISRDNSKNTLYLQMNSLRAEDTAVYYCASIAVAGNYFDYWGQGTLVTVSSGGGGSGGGGSGGGGSSYVLTQDPAVSVALGQTVRITCQGDSLRSYYASWYQERPGQAPLLVIYGKNNRPSGIPDRFSGSNSGSTATLTISRVEAGDEGDYYCQVWDSINEQVVFGGGTKVTVL

SEQ ID NO: 172 - protein, 241 aa, Artificial Sequence
  Description of Artificial Sequence Synthetic polypeptide
QVQLVQSGGGVVQPGRSLRLSCAATGIPFSGSGMHWVRQAPGKGLEWVTMIWYDGSNKFYADSVKGRFTISRDNSKNTLYLQMDSLRAEDTAVYFCARDKGVRSMDVWGLGTTVTVSSGGGGSGGGGSGGGGSNFMLTQPPSVSVAPGQTAKITCDGYSIRTKSVHWYQQKPGQAPVVVVHDDSDRPSGIPERFSGSNSGTTATLTISRVEAGDEADYYCQAWDSISEEVVFGGGTKLTVL

SEQ ID NO: 173 - protein, 240 aa, Artificial Sequence
  Description of Artificial Sequence Synthetic polypeptide
QVQLQESGGGLVQPGGSLRLSCSASGFTFGTYAMRWVRQTSGKGLEWVSGIGVSGDAYYTDSVRGRFTISRDNSKNTLYLQMNTLRAEDTATYYCTRKSSTTSNDYWGRGTLVTVSSGGGGSGGGGSGGGGSSYVLTQDPAVSVALGQTVRITCQGDNIGSKSVHWYQQKPGQAPVLVVYDDSDRPSGIPERFSGSNSGTTATLTISSVEAGDEADYYCQAWDSISEHVIFGGGTKVTVL

SEQ ID NO: 174 - protein, 241 aa, Artificial Sequence
  Description of Artificial Sequence Synthetic polypeptide
QVQLQESGGGLVQPGGSLRLSCAASGFTFSSYAMHWVRQAPGKGLEWVAVISYDGSNKYYADSVKGRFTISRDNSKNTLYLQMNSLRAEDTAVYYCARFSSGWYYFDYWGQGTLVTVSSGGGGSGGGGSGGGGSDIQMTQSPSFLSASVGDRITITCRASHDISSYFAWYQQKPGKAPKPLIYAASTLQSGVPSRFSGSGSGTEFTLTISSLQPEDFATYYCQQLGSYPLTFGGGTKLEIK

SEQ ID NO: 175 - protein, 245 aa, Artificial Sequence
  Description of Artificial Sequence Synthetic polypeptide
QVQLLQSGGGLVQPGGSLRLSCAASGFTFSSYAMSWVRQAPGKGLEWVSAISGSGGSTYYTDSVKGRFTISRDNSKNTLYLQMNSLRAEDTAVYYCAKSHDYGDYAGFDYWGQGTLVTVSSGGGGSGGGGSGGGGSHVILTQDPAVSVALGQTVRITCQGDSLKSYYASWYQQKPGQAPVLVIYGKNNRPSGIPDRFSGSSSGTTASLTITGAQAEDEADYYCHSRDSSGTHLRVFGGGTKLTVL

SEQ ID NO: 176 - protein, 244 aa, Artificial Sequence
  Description of Artificial Sequence Synthetic polypeptide
QVQLQESGGGLVKPGGSLRLSCAASGFTFSNAWMNWVRQAPGKGLEWVGRIKSKTDEGTTDYAAPVKGRFSISRDDSKNTLYLQMNSLKTEDTGVYYCTATKGLGGSKLGQGTLVTVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 177 - protein, 245 aa, Artificial Sequence
  Description of Artificial Sequence Synthetic polypeptide
QVQLVESGGGVVQPGRSLRLSCAASGFTFSSFGMHWVRRAPGKGLEWVAVISYDGSNQYYADSVKGRFTISRDNSKNTLYLQMNSLRAEDTAVYYCGSRPGGGYASGSTVAYWGQGTPVTVSSGGGGSGGGGSGGGGSSSELTQDPAVSVALGQTVRITCQGDSLRSYYASWYQQKPGQAPLLVIYGQNIRPSGIPDRFSGSSSGNSASLTITGAQAEDEADYYCHSRDSSGKYVFGVGTKVTVL

SEQ ID NO: 178 - protein, 237 aa, Artificial Sequence
  Description of Artificial Sequence Synthetic polypeptide
QVQLVESGGGLVQPGGSLRLSCAASGFTFSSYAMGWVRQAPGKGLEWVSAISGSGGSTYYADSVKGRFTISRDNSKDTLYLQMNSLRAEDTAVYYCASRSLLDYWGQGTLVTVSSGGGGSGGGGSGGGGSNFMLTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKVTVL

SEQ ID NO: 179 - protein, 237 aa, Artificial Sequence
  Description of Artificial Sequence Synthetic polypeptide
QVQLVESGGGLVQPGGSLRLSCAASGFTFSSYAMSWVRQAPGKGLEWVSAISGSGGSTYYADSVKGRFTISRDNSKDTLYLQMNSLRAEDTAVYYCASRSLLDYWGQGTLVTVSSGGGGSGGGGSGGGGSNFMLTQDPAVSVALGQTVRITCQGDSLRSYYASWYQQKPGQAPLLVIYGKNNRPSGIPDRFSGSSSGNTASLTITGAQAEDEADYYCNSRDSSGNPVFGGGTKVTVL

SEQ ID NO: 180 - protein, 243 aa, Artificial Sequence
  Description of Artificial Sequence Synthetic polypeptide
QVQLQESGGGLVQLGGSLRLSCAASGFTFSSYAMSWVRQAPGKGLEWVSAISGSGGSTYYADSVKGRFTISRDNSKNTLYLQMSSLRAEDTAFYYCANSAYTGGWYDYWGHGTLVTVSSGGGGSGGGGSGGGGSSSELTQDPAVSVALGQTVKITCQGDSLRTYYASWYQQRPGQAPVLVIYGENSRPSGIPDRFSGSSSGNTASLTITGAQAEDEADYYCNSRDSSGNHLRVFGGGTKLTVL

SEQ ID NO: 181 - protein, 238 aa, Artificial Sequence
  Description of Artificial Sequence Synthetic polypeptide
QVNLRESGGGLVQPGGFLRLSCAAFGFTFSGYWMSWVHPAPGKGLEWVANIKQDGSEKFYVDSVKGRFTISRDNAKNSLFLQMNSLRAEDTAVYFCARGLLSDYWGQGTLVPVSSGGGGSGGGGSGGGGSNFMLTQPPSVSVAPGKTASLTCGGYNIGTKSVHWYQQKPGQAPVVVVHDDSDRPSGIPERFSGSNSGTTATLTIIRVEAGDEADYYCQAWDSISEEVVFGGGTKLTVL

SEQ ID NO: 182 - protein, 242 aa, Artificial Sequence
  Description of Artificial Sequence Synthetic polypeptide
QVQLQESGGGLVKPGGPLRLSCAASGFTFSSYGMYWVRQAPGKGLEWVSTLSRSGSGTYYAESVKGRFTISRDNSKNTLYFQMNSLRAEDTAVYYCASIAVAGNYFEYWGQGTLVTVSSGGGGSGGGGSGGGGSSYVLTQDPAVSVALGQTVRITCQGDSLRSYYASWYQERPGQAPLLVIYGKNNRPSGIPDRFSGSNSGSTATLTISRVEAGDEGDYYCQVWDSINEQVVFGGGTKVTVL

SEQ ID NO: 183 - protein, 243 aa, Artificial Sequence
  Description of Artificial Sequence Synthetic polypeptide
QVQLQESGGGLVKPGESLRLSCAASGFTFSDHYMDWVRQAPGKGLEWVAYIRYDGSTKYYADSVKGRFTISRDNSKNTLYLQMNSLRPEDTAFYYCARLIAEAEGWFDPWGQGTLVTVSSGGGGSGGGGSGGGGSNFMLTQPPSVSVAPGKTARITCGGNNIGSKSVYWYQQKPGQAPVLVVYDDSDRPSGIPERFSGSNSGNTATLTISRVEAGDEADYYCQVWDSSSDHVVFGGGTKVTVL

SEQ ID NO: 184 - protein, 241 aa, Artificial Sequence
  Description of Artificial Sequence Synthetic polypeptide
QVQLLQSAGGLVQPGGSLRLSCAASGFTFSTYAMNWVRQAPGKGLEWVSGISGSGGSTNYADSVKGRFTISRDSSKNTLFLQMNSLRAEDTAVYYCAKDYGSGWYDYWGQGTLVTVSSGGGGSGGGGSGGGGSSSELTQDPAVSVALGQTVRITCQGDSLRSYYASWYQERPGQAPLLVIYGRNERPSGIPDRFSASSSGNTASLTITGAQAEDEADYYCQVWDSFNEQVVFGGGTKLTVL

SEQ ID NO: 185 - protein, 245 aa, Artificial Sequence
  Description of Artificial Sequence Synthetic polypeptide
QVQLVESGGGLVQPGGSLRLSCAASGFTFSSYAMSWVHQAPGKGLEWVSAISGSGGSTYYADSVKGRFTISRDNSKNTLYLQMNSLRAEDTAVYYCAKTYYGFWSGYYDYLGQGTLVTVSSGGGGSGGGGSGGGGSSSELTQDPAVSVGLGQTVTITCQGDSLRSYYANWYQQKPGQAPILVIYGENNRPSGIPDRFSGSSSGNTASLTITGAQAEDEADYYCHSRDSSGTHLRVFGGGTKLTVL

SEQ ID NO: 186 - protein, 244 aa, Artificial Sequence
  Description of Artificial Sequence Synthetic polypeptide
QVQLLESGGGVVQPGTSLRLSCAASGFTFSNYAINWVRQAAGKGLEWVSGISGSGVSTSYADSVKGRFTVSRDNSKNTLYLQMNSLRVEDTALYYCAKNGGGPEYLQHWGQGTLVTVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 187 - protein, 238 aa, Artificial Sequence
  Description of Artificial Sequence Synthetic polypeptide
QVQLQESGGTLVQPGGSLRLSCAASGFTFSSYAMSWVRQAPGRGLEWVSTISGSGGSTYYADSVKGRFTISRDNSKNTLYLQMNSLRAEDTAVYYCAKGAYSGSYWGQGTLVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 188 - protein, 238 aa, Artificial Sequence
  Description of Artificial Sequence Synthetic polypeptide
QVQLQESGGTLVQPGGSLRLSCAASGFTFSSYAMSWVRQAPGRGLEWVSTISGSGGSTYYADSVKGRFTISRDNSKNTLYLQMNSLRAEDTAVYYCAKGAYSGSHWGQGTLVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 189 - protein, 244 aa, Artificial Sequence
  Description of Artificial Sequence Synthetic polypeptide
QVQLVESGAEVKKPGASVKVSCKASGYTFTSYGISWVRQAPGQGLEWMGWISAYNGNTNYAQKLQGRVTMTTDTSTSTAYMELRSLRSDDTAVYYCARPIYDSSGYDAFDIWGQGTMVTVSSGGGGSGGGGSGGGGSDIVMTQSPSTLSASIGDRVTITCRASEGIYHWLAWYQQKPGKAPKLLIYKASSLASGAPSRFSGSGSGTDFTLTISSLQPDDFATYYCQQYHTISRTFGPGTKVDIK

SEQ ID NO: 190 - protein, 244 aa, Artificial Sequence
  Description of Artificial Sequence Synthetic polypeptide
QVQLVESGGGLVKPGGSLRLSCAASGFTFSSYAMHWVRQAPGKGLEWVAVISYDGSNKYYADSVKGRFTISRDNSKNTLYLQMNSLRAEDTAVYFCVRPSDSGWSFEHWGQGTLVPVSSGGGGSGGGGSGGGGSQSVLTQPPSASGTPGQRVTISCSGSSSNIGNNTVNWSRQLPGTAPKLLIYSNDQRPSGVPDRFSGSKSGTSASLAITGLQPEDEADYYCGTWDSSLSAYVFGTGTKLTVL

SEQ ID NO: 191 - protein, 245 aa, Artificial Sequence
  Description of Artificial Sequence Synthetic polypeptide
QVQLQESGGGLVQPGGSLRLSCAASGFTFSSYAMSWVRQAPGKGLEWVAVISYDGSNKYYADSVKGRFTISRDNSKNTLYLQMNSLRAEDTAVYYCVRGDRSYGAEYFQHWGQGTLVTVSSGGGGSGGGGSGGGGSSSELTQDPAVSVASGQTVRITCQGDSLRSYYASWYQQKPGQAPLLVIYGKNIRPSGIPDRFSGSTSGNSASLTITGAQAEDEADYYCNSRDSSGNRNWVFGGGTKLTVL

SEQ ID NO: 192 - protein, 246 aa, Artificial Sequence
  Description of Artificial Sequence Synthetic polypeptide
QVQLQESGGGLVKPGGSLRLSCAASGFTSSSYAMHWVRQAPGKGLEYVSAIGGNGGTYYADSVKGRFTISRDNSKNTLYLQMNSLRAEDTAVYYCAKEGEQWLEYRYYYGMDVWGQGTTVTVSSGGGGSGGGGSGGGGSSSELTQDPAVSVALGQTVRITCQGDSLRSYYASWYQQKPGQAPSLVIYGENSRPSGIPDRFSGSSSGNTASLTITGAQAENEADYYCQAWDSSTAVVFGGGTKLTVL

SEQ ID NO: 193 - protein, 250 aa, Artificial Sequence
  Description of Artificial Sequence Synthetic polypeptide
QVQLVESGGGVVQPGRSLRLSCTASGFTFSSYGMHWVRQAPGKGLEWVAVISYDGSNKYYADSVKGRFTISRDNSKNTLYLQMNSLRAEDTAVYYCARGGRYSSNWFSYYYYGMDVWGQGTTVTVSSGGGGSGGGGSGGGGSNFMLTQPPSVSVAPGKTARITCGGNNIGSKSVYWYQQKPGQAPVLVVYDDSDRPSGIPERFSGSNSGNTATLTISRVEAGDEADYYCQVWDSSSDHVVFGGGTKVTVL

SEQ ID NO: 194 - protein, 15 aa, Artificial Sequence
  Description of Artificial Sequence Synthetic peptide
GGGGSGGGGSGGGGS

SEQ ID NO: 195 - protein, 10 aa, Artificial Sequence
  Description of Artificial Sequence Synthetic peptide
GGGGSGGGGS

SEQ ID NO: 196 - protein, 5 aa, Artificial Sequence
  Description of Artificial Sequence Synthetic peptide
GGGGS

SEQ ID NO: 197 - protein, 20 aa, Artificial Sequence
  Description of Artificial Sequence Synthetic peptide
GSGGGGSGGGGSGGSGGGGS

SEQ ID NO: 198 - protein, 6 aa, Artificial Sequence
  Description of Artificial Sequence Synthetic peptide
SGGGGS

SEQ ID NO: 199 - protein, 4 aa, Artificial Sequence
  Description of Artificial Sequence Synthetic peptide
GGGS

SEQ ID NO: 200 - protein, 4 aa, Artificial Sequence
  Description of Artificial Sequence Synthetic peptide
VPGV

SEQ ID NO: 201 - protein, 5 aa, Artificial Sequence
  Description of Artificial Sequence Synthetic peptide
VPGVG

SEQ ID NO: 202 - protein, 6 aa, Artificial Sequence
  Description of Artificial Sequence Synthetic peptide
GVPGVG

SEQ ID NO: 203 - protein, 8 aa, Artificial Sequence
  Description of Artificial Sequence Synthetic peptide
GVGVPGVG

SEQ ID NO: 204 - protein, 10 aa, Artificial Sequence
  Description of Artificial Sequence Synthetic peptide
VPGVGVPGVG

SEQ ID NO: 205 - protein, 7 aa, Artificial Sequence
  Description of Artificial Sequence Synthetic peptide
GGSSRSS

SEQ ID NO: 206 - protein, 18 aa, Artificial Sequence
  Description of Artificial Sequence Synthetic peptide
GGSSRSSSSGGGGSGGGG